(19) United States
(12) Patent Application Publication
(10) Pub. No.: US 2024/0174975 A1
(43) Pub. Date: May 30, 2024

(54) Modular DNA Nanoshells for Cell Encapsulation and Ruggedization

(71) Applicant: Carnegie Mellon University, Pittsburgh, PA (US)

(72) Inventors: Rebecca Taylor, Pittsburgh, PA (US); Weitao Wang, Pittsburgh, PA (US); Xi Ren, Wexford, PA (US)

(21) Appl. No.: 18/462,634

(22) Filed: Sep. 7, 2023

Related U.S. Application Data
(60) Provisional application No. 63/404,379, filed on Sep. 7, 2022.

(51) Int. Cl. C12N 5/00; C12N 5/074

(52) U.S. Cl. CPC C12N 5/0006; C12N 5/0012; C12N 5/0607

(57) ABSTRACT

Ruggedized particles or live cells are provided. The particles or cells comprise a cross-linked matrix of nucleic acid and/or nucleic acid analog nanostructures forming a shell about the particle or cell. Methods of making and using the ruggedized particles or live cells are provided. The ruggedized particles or cells may be decorated with environmental sensors, for example, which are prepared from nucleic acid and/or nucleic acid analog nanostructures and may include a FRET pair.

### CROSS REFERENCE TO RELATED APPLICATIONS

This application claims priority to U.S. Provisional Patent Application No. 63/404,379 filed Sep. 7, 2022, the disclosure of which is incorporated herein by reference in its entirety.

### STATEMENT REGARDING FEDERAL FUNDING

This invention was made with U.S. government support under FA9550-18-1-0199 awarded by the Air Force Office of Scientific Research. The government has certain rights in the invention.

### SEQUENCE LISTING

The Sequence Listing associate with this application is filed in electronic format via Patent Center and is hereby incorporated by reference into the specification in its entirety. The name of the XML file containing the Sequence Listing is 06526-2306008.xml. The size of the XML filed is 167,936 bytes and the XML file was created on Sep. 6, 2023.

The cellular plasma membrane serves as a protective barrier by encapsulating cellular components. This biomembrane is decorated with membrane-bound proteins, making it essential for mediating cellular signaling and sensing. The plasma membrane is also linked to the interior cytoskeleton that mechanically supports the cell to maintain its size, shape and integrity. It therefore allows for cellular communication while shielding the cell from outside assaults. However, the cell membrane is often unable to protect the cell from external stressors, for example, the high forces and subsequent large membrane deformations experienced during cell manipulation and delivery applications in tissue engineering and regenerative medicine.

Cell encapsulation is recognized as one approach to tackle this problem with various nanomaterials being extensively investigated to wrap the whole cell for cellular protection and manipulation. However, the lack of material programmability limits the control over the encapsulation, such as the tunability of the encapsulation formation and its on-demand removal. Moreover, material overload as well as the cytotoxic nature of certain materials may hinder cell function and even lead to cell death.

As such, ruggedized cells and methods of ruggedizing cells are needed that retain the viability of the ruggedized cells.

### SUMMARY

According to a first aspect or embodiment, a ruggedized particle or cell is provided, comprising: a particle or live cell; and a shell comprising a plurality of first nucleic acid and/or nucleic acid analog nanostructures anchored to the particle or cell; and a plurality of second nucleic acid and/or nucleic acid analog nanostructures cross-linking the plurality of first nucleic acid and/or nucleic acid analog nanostructures by a plurality of nucleic acid and/or nucleic acid analog staples.

According to a second aspect or embodiment, a method of preparing a ruggedized particle or cell is provided, comprising: anchoring a plurality of first nucleic acid and/or nucleic acid analog nanostructures to a surface of a particle or a live cell, and forming a shell by crosslinking the plurality of first nucleic acid and/or nucleic acid analog nanostructures with a plurality of second nucleic acid and/or nucleic acid analog nanostructures using a plurality of nucleic acids and/or nucleic acid analog staples, wherein the plurality of first nucleic acid and/or nucleic acid analog nanostructures are attached to the plurality of second nucleic acid and/or nucleic acid analog nanostructures.

According to a third aspect or embodiment, a method of preparing a graft for tissue replacement or repair in a patient is provided, comprising: depositing a ruggedized cell as described above onto the surface of a polymer matrix structure; culturing the ruggedized cells on the polymer matrix structure under conditions for differentiating the cells into cells of the tissue to be repaired or replaced; and culturing the ruggedized cells to expand the number of cells on the polymer matrix structure.

According to a fourth aspect or embodiment, an environmental sensing method is provided, comprising, introducing the ruggedized particle or cell described herein comprising an environmental sensor to a sensing environment, and determining the effect of the environment on the sensor.

According to a first aspect or embodiment, a ruggedized particle or cell is provided, comprising:

The following numbered clauses describe certain exemplary aspects or embodiments of the present invention:

Clause 1. A ruggedized particle or cell comprising: a particle or live cell; and a shell comprising a plurality of first nucleic acid and/or nucleic acid analog nanostructures anchored to the particle or cell; and a plurality of second nucleic acid and/or nucleic acid analog nanostructures cross-linking the plurality of first nucleic acid and/or nucleic acid analog nanostructures by a plurality of nucleic acid and/or nucleic acid analog staples.

Clause 2. The ruggedized particle or cell of clause 1, wherein the particle or live cell is a live cell.

Clause 3. The ruggedized particle or cell of clause 1, wherein the particle or live cell is a lipid-based nanoparticle, a lipid-based nanocarrier, an inorganic nanoparticle, or a polymeric nanoparticle.

Clause 4. The ruggedized particle or cell of clause 1, wherein the particle or live cell is a nanoemulsion, a solid lipid nanoparticle, a phospholipid micelle, a liposome, a vesicle, an exosome, a giant unilamellar vesicle, an iron oxide nanoparticle, a gold nanoparticle, a carbon allotrope particle, a PLGA (poly(lactic-co-glycolic acid)) particle, an m PEG-PGA (methoxy-poly(ethyleneglycol)-block-poly(L-glutamic acid)) particle; a PEG-PMT (PEG-poly(ω-penta-decalactone-co-N-methyldiethyleneamine sebacate-co-2,2′-thiodiethylene sebacate) particle, or a PEI-PLGA (polyethyleneimine-PLGA) particle.

Clause 5. The ruggedized particle or cell of clause 2, comprising:
the live cell;
a plurality of first nucleic acid and/or nucleic acid analog nanostructure beams anchored to the cell, the beams comprising one or more single-stranded scaffold nucleic acids or nucleic acid analogs;
nucleic acids and/or nucleic acid analog staples attached to the plurality of first nucleic acid and/or nucleic acid analog nanostructure beams and nucleic acids and/or nucleic acid analog staples attached to the plurality of second nucleic acid and/or nucleic acid analog nanostructures, wherein the nucleic acids and/or nucleic acid analog staples attached to the plurality of second nucleic acid and/or nucleic acid analog nanostructures are complementary to the nucleic acids and/or nucleic acid analog staples attached to the plurality of first nucleic acid and/or nucleic acid analog nanostructure beams; and
the plurality of second nucleic acid and/or nucleic acid analog nanostructures linked to the plurality of first nucleic acid and/or nucleic acid analog nanostructure beams by the staples and which cross-link two or more of the first nucleic acid and/or nucleic acid analog nanostructures to the second nucleic acid and/or nucleic acid analog nanostructures.

Clause 6. The ruggedized particle or cell of clause 2, wherein the plurality of first nucleic acid and/or nucleic acid analog nanostructures are anchored to the glycocalyx of the cell.

Clause 7. The ruggedized particle or cell of clause 2, wherein the plurality of first nucleic acid and/or nucleic acid analog nanostructures are anchored to a surface protein of the cell, such as a receptor.

Clause 8. The ruggedized particle or cell of any one of clauses 5-7, wherein the plurality of first nucleic acid and/or nucleic acid analog nanostructures are anchored to the surface protein of the cell or to the glycocalyx of the cell by a click chemistry reaction, and optionally a biorthogonal click chemistry method.

Clause 9. The ruggedized particle or cell of clause 1 or 5, wherein the plurality of first nucleic acid and/or nucleic acid analog nanostructures are linked to a hydrophilic moiety and are anchored to the lipid bilayer membrane of the cell via the hydrophobic moiety.

Clause 10. The ruggedized particle or cell of clause 9, wherein the hydrophobic moiety comprises one or more fatty acid or cholesterol moieties.

Clause 11. The ruggedized particle or cell of clause 10, wherein the hydrophobic moiety comprises a glycosylphosphatidylinositol (GPI) moiety.

Clause 12. The ruggedized particle or cell any one of clauses 1-11, wherein the plurality of second nucleic acid and/or nucleic acid analog nanostructures comprise nucleic acid and/or nucleic acid analog nanostructure beams.

Clause 13. The ruggedized particle or cell of any one of clauses 1-12, wherein the beams of the first nucleic acid and/or nucleic acid analog nanostructures are comprise from two to ten rods (e.g., helical nucleic acid and/or nucleic acid analog bundles), such as six rods.

Clause 14. The ruggedized particle or cell of clause 13, wherein the beams of the second nucleic acid and/or nucleic acid analog nanostructures comprise from two to ten rods (e.g., helical nucleic acid and/or nucleic acid analog bundles), such as six rods.

Clause 15. The ruggedized particle or cell of clause 13, wherein the rods of the first nucleic acid and/or nucleic acid analog nanostructures are anchored at an end to the cell.

Clause 16. The ruggedized particle or cell of clause 2, wherein the cell is a multipotent cell or a pluripotent cell such as a stem cell, e.g., a mesenchymal stem cell, a cell expressing a recombinant protein or RNA, an immune system cell, such as an antigen-presenting cell, a lymphocyte, a neutrophil, or a monocyte, or a platelet.

Clause 17. The ruggedized particle or cell of any one of clauses 1-16, wherein the plurality of first nucleic acid and/or nucleic acid analog nanostructures form a first layer about the cell, and the plurality of second nucleic acid and/or nucleic acid analog nanostructures form a second layer over the first layer.

Clause 18. The ruggedized particle or cell of any one of clauses 1-17, further comprising a nucleic acid and/or nucleic acid analog nanostructure environmental sensor attached to the shell.

Clause 19. The ruggedized particle or cell of clause 18, wherein the environmental sensor comprises a fluorophore or a FRET pair.

Clause 20. A method of preparing a ruggedized particle or cell, comprising anchoring a plurality of first nucleic acid and/or nucleic acid analog nanostructures to a surface of a particle or a live cell, and forming a shell by crosslinking the plurality of first nucleic acid and/or nucleic acid analog nanostructures with a plurality of second nucleic acid and/or nucleic acid analog nanostructures using a plurality of nucleic acids and/or nucleic acid analog staples, wherein the plurality of first nucleic acid and/or nucleic acid analog nanostructures are attached to the plurality of second nucleic acid and/or nucleic acid analog nanostructures.

Clause 21. The method of clause 20, further comprising:
forming a plurality of first nucleic acid and/or nucleic acid analog nanostructure beams comprising the plurality of nucleic acids and/or nucleic acid analog staples, the beams comprising one or more single-stranded scaffold nucleic acids or nucleic acid analogs;
anchoring the plurality of first nucleic acid and/or nucleic acid analog nanostructure beams comprising the plurality of nucleic acids and/or nucleic acid analog staples to the surface of the particle or cell;
forming the plurality of second nucleic acid and/or nucleic acid analog nanostructures comprising a plurality of nucleic acids and/or nucleic acid analog staples, wherein the nucleic acids and/or nucleic acid analog staples of the plurality of second nucleic acid and/or nucleic acid analog nanostructures are complementary to the plurality of nucleic acids and/or nucleic acid analog staples of the plurality of first nucleic acid and/or nucleic acid analog nanostructure beams;
cross-linking the plurality of first nucleic acid and/or nucleic acid analog nanostructure beams and the plurality of second nucleic acid and/or nucleic acid analog nanostructures through hybridization of the staples to link two or more of the first nucleic acid and/or nucleic acid analog nanostructure beams to the second nucleic acid and/or nucleic acid analog nanostructures to form the shell.

Clause 22. The method of clause 20 or 21, wherein the plurality of first nucleic acid and/or nucleic acid analog nanostructures comprise nucleic acid and/or nucleic acid analog nanostructure beams.

Clause 23. The method of clause 20 or 21, wherein the second nucleic acid and/or nucleic acid analog nanostructures comprise nucleic acid and/or nucleic acid analog nanostructure beams.

Clause 24. The method of any one of clauses 22 or 23, wherein the nucleic acid and/or nucleic acid analog nanostructure beams comprise from two to ten rods (e.g., helical nucleic acid analog bundles), such as six rods.

Clause 25. The method of clause 24, wherein the rods of the first nucleic acid and/or nucleic acid analog nanostructure beams are anchored at an end to the particle or cell.

Clause 26. The method of any one of clauses 20-25, wherein the particle or cell is a live cell.

Clause 27. The method of clause 26, where the plurality of first nucleic acid and/or nucleic acid analog nanostructures are anchored to the glycocalyx of the cell.

Clause 28. The method of clause 26, wherein the plurality of first nucleic acid and/or nucleic acid analog nanostructures are anchored to a surface protein of the cell, such as a receptor.

Clause 29. The method of clause 26, wherein the plurality of first nucleic acid and/or nucleic acid analog nanostructures are anchored to a surface protein of the cell or to the glycocalyx of the cell by a click chemistry reaction, and optionally a biorthogonal click chemistry method.

Clause 30. The method of clause 26, wherein the plurality of first nucleic acid and/or nucleic acid analog nanostructures are linked to a hydrophilic moiety and are anchored to the lipid bilayer membrane of the cell via the hydrophobic moiety.

Clause 31. The method of clause 30, wherein the hydrophobic moiety comprises one or more fatty acid or cholesterol moieties.

Clause 32. The method of clause 31, wherein the hydrophobic moiety comprises a glycosylphosphatidylinositol (GPI) moiety.

Clause 33. The method of any one of clauses 26-32, wherein the cell is a multipotent cell or a pluripotent cell such as a stem cell, e.g., a mesenchymal stem cell, a cell expressing a recombinant protein or RNA, an immune system cell, such as an antigen-presenting cell, a lymphocyte, a neutrophil, or a monocyte, or a platelet.

Clause 34. The method of any one of clauses 20-33, wherein forming the shell comprises:
forming a first layer about the particle or cell with the plurality of first nucleic acid and/or nucleic acid analog nanostructures, and
forming a second layer over the first layer with the plurality of second nucleic acid and/or nucleic acid analog nanostructures.

Clause 35. The method of any one of clauses 20-34, further comprising attaching a nucleic acid and/or nucleic acid analog nanostructure environmental sensor to the shell.

Clause 36. The method of clause 35, wherein the environmental sensor comprises a fluorophore or a FRET pair.

Clause 37. A method of preparing a graft for tissue replacement or repair in a patient, the method comprising:
depositing a ruggedized cell as described in clause 2 onto the surface of a polymer matrix structure;
culturing the ruggedized cells on the polymer matrix structure under conditions for differentiating the cells into cells of the tissue to be repaired or replaced; and
culturing the ruggedized cells to expand the number of cells on the polymer matrix structure.

Clause 38. The method of clause 37, wherein the ruggedized cell is deposited through an electrospray deposition method or a printing method.

Clause 39. An environmental sensing method comprising, introducing the ruggedized particle or cell of clause 18 to a sensing environment, and determining the effect of the environment on the sensor.

Clause 40. The method of clause 39, wherein the environmental sensor is a force sensor.

Clause 41. The method of clause 40, wherein the force sensor comprises one or more FRET pairs that fluoresce differently depending on mechanical forces applied to the cell.

### BRIEF DESCRIPTION OF THE DRAWINGS

FIG. 1 provides structures of exemplary nucleic acid analogs.

FIG. 2 provides structures of exemplary nucleobases.

FIGS. 3A-3B provide structures of exemplary nucleobases.

FIGS. 4A-4C provide structures of exemplary nucleobases.

FIG. 5 shows the design of 3-rod (e.g., helix) γPNA beams. FIG. 5(a) is a schematic comparison between a full helical turn of DNA and PNA double helix. FIG. 5(b) is a schematic representation of the structural motif for the SST design that shows 6-base domains and an overall repeat unit of 18 bases. FIG. 5(c) shows melt curve studies. FIG. 5(d) shows how 6-base domains correspond to 120° rise in helical rotation enabling the structural motif to program the assembly of 3-rod (e.g., helix) beams that can polymerize lengthwise.

FIG. 6 depicts an exemplary four-helix ss-γPNA SST structure.

FIG. 7 depicts an exemplary six-helix ss-γPNA SST structure.

FIG. 8 is a graph of DNA oligos for DNA nanorods.

FIGS. 9A-9D depict the schematics of the nanoshell synthesis process and validation of stability. FIG. 9A depicts the synthesis of the DNA nanoshell on the cellular membrane through a three-step immobilization process including: (i) the immobilization of a′-ssDNA initiators on the glycocalyx; (ii) the binding of Rod A to a′-ssDNA via ssDNA hybridization and (iii) the binding and crosslinking of Rod B to Rod A via the hybridization of h-ssDNA on rod A and h′-ssDNA on Rod B. Atomic Force Microscopy imaging of the rods were shown. Scale bar: 500 nm. FIG. 9B shows an agarose gel electrophoresis of individual DNA rods, and a mixture of rods after a 30 minute incubation of Rod A and Rod B at 37° C. FIG. 9C shows an agarose gel electrophoresis study of the stability of individual DNA rods and the aggregate in two types of cell culture medium. Rod A and Rod A and Rod B mixtures were incubated at 37° C. for 6 hours and 30 hours in each cell culture medium. FIG. 9D shows a schematic for the immobilization of DBCO-labeled a′-ssDNA initiators on azide-presenting cell-surface glycocalyx through copper free click chemistry.

FIGS. 10A-10F depicts the modulation of the DNA nanoshell composition by engineering the position and number of overhangs on rods. FIG. 10A is fluorescence imaging and intensity quantification of four experimental conditions including: (i) Rod A (biotin) coating with a′-ssDNA initiators on the glycocalyx; (ii) Rod A (biotin) coating without a′-ssDNA initiators; (iii) Rod B (biotin) coating with both the initiators and Rod A; and (iv) Rod B (biotin) coating with initiators but without Rod A. The +/− symbols and accompanying bar graphs indicated whether Rod A or Rod B populations, respectively, were fluorescently labeled. FIG. 10B is the normalized fluorescence intensity profiles of nanoshell-coated cells with Cy3-staining rod A and Cy5-staining rod B. FIG. 10C shows the gel electrophoresis studies for the quantification of the percentage of aggregate resulting from each mixture of DNA Rod A and Rod B as a function of the number of hybridization overhangs on rods. FIG. 10D shows the modulation of the binding of Rod B to Rod A by changing the number of h′-ssDNA on Rod B. FIG. 10E shows the modulation of the binding of Rod B to Rod A by changing the number of h-ssDNA on Rod A. FIG. 10F shows the modulation of the binding of Rod A onto the cell membrane and Rod B to Rod A by changing the position of a-ssDNA on Rod A. The fluorescence intensity quantification of four conditions includes: (i) edge-decorated Rod A (biotin); (ii) center-decorated Rod A (biotin); (iii) edge-decorated Rod A and Rod B (biotin); and (iv) center-decorated Rod A and Rod B (biotin). Schematics of the rods and the representative fluorescence microscope images are shown. Data were presented as means±s.d. in FIGS. 10A and 10D-10F, **P≤0.01, ***P0.001. All scale bars: 10 μm.

FIGS. 11A and 11B show the modulation of nanoshell composition by the concentrations of DNA rods. FIG. 11A is a graph of the fluorescence intensity (labeled with AF-647) of the binding of Rod A by adjusting the concentration of Rod A from 1 nM, 5 nM to 10 nM. FIG. 11B is a graph of the fluorescence intensity of the binding of Rod B to Rod A after 5 nM of rod A was first administrated to cells and then Rod B was introduced where the concentration of Rod B was adjusted from 1 nM, 5 nM to 10 nM. ***P≤0.001 (n=5).

FIGS. 12A-12F show the cell surface retention time, cellular internalization, and morphology of the DNA nanoshell. FIG. 12A is a graph of the distribution of cell populations versus AF647 fluorescence intensity to show the cell surface retention time of DNA rods measured by fluorescence-activated cell sorting (FACS). FIG. 12B are fluorescence images and the quantification on the cellular uptake of DNA Rod A on Rod A-coated cells and nanoshell-coated cells using HUVECs. FIG. 12C are plots showing the analysis of DNA rod distribution on the borders of cell membranes. The fluorescence intensity along the cell borders was tracked and plotted for rod A-coated cells and nanoshell-coated cells after 0 hours and 3 hours incubation. Wide-field fluorescence microscopy images are included and show the distribution of fluorescence signal on cells. Signal valleys are shown and marked in the plots. The signal valley quantification of FIG. 12C was reported in terms of the number (FIG. 12D) and the length (FIG. 12E) of fluorescence signal valleys in each group. n=10. *P≤0.05, **P≤0.01, ***P≤0.001. FIG. 12F includes confocal microscopy cross-section images and reconstructed three-dimensional models of representative Rod A-coated cells and nanoshell-coated cells. All scale bars: 10 μm.

FIGS. 13A and 13B depict DNA nanorods and DNA nanoshell degradation by DNase I. FIG. 13A is a graph of the normalized fluorescence signal intensity of Rod A on Rod A-coated cells before and after DNase I treatment. FIG. 13B is a graph of the normalized fluorescence signal intensity of Rod A and Rod B on nanoshell-coated cells before and after DNase I treatment. ***P≤0.001.

FIGS. 14A-14F depict the tunable modulation of the biophysical properties of cell membranes by the DNA origami nanoshell. FIG. 14A) is a representative image of micropipette aspiration with a cell being aspirated into a micropipette with an inner radius Rp=2.5 μm under an aspiration pressure ΔP. FIG. 14B is a graph showing the relationship between the ΔP and the normalized aspiration length Lₚ/Rₚ. The datapoints represent cells including native cells, rod A-coated cells, and nanoshell-coated cells with rods with 1, 3 and 14 h/h′-ssDNA. FIG. 14C is a graph showing the evaluation of membrane elastic modulus (Theret et al. “The Application of a Homogeneous Half-Space Model in the Analysis of Endothelial Cell Micropipette Measurements”, Journal of Biomechanical Engineering, 1988, 110:190-199). For FIGS. 14B and 14C, n=7. FIG. 14D shows FRAP experiments and the normalized membrane lipid fluorescence intensity over time after photobleaching a region of interest on the membranes of cells with different rod coating. Data are presented as means (solid lines) ±s.d. (shadow areas) (n=10). FIG. 14E is a graph of the evaluation of the mobile fraction of membrane lipid and FIG. 14F is a graph of the half-time recovery of membrane lipid of native cells, rod A-coated cells and nanoshell-coated cells (rods with 1, 3 and 14 h/h′-ssDNA). For FIGS. 14E and 14F, n=30. *P≤0.05, ***P≤0.001. Allscale bars: 10 μm. FIGS. 15A-15B depict the fluorescence recovery after photobleaching (FRAP) experiments of native cells, Rod A-coated cells, and nanoshell-coated cells. FIG. 15A is a graph of the recovery constant of cell membrane lipids. FIG. 15B is a graph of the mobility of DBCO-cy5 in native cell+DBCO-cy5, the mobility of Rod A in Rod A-coated cells, and the mobility of Rod A in nanoshell-coated cells. For nanoshell-coated cells, two conditions were included, where the rods were decorated with 1, 3, and 14 h/h′-ssDNA. n=30. *P≤0.05, ***P≤0.001.

FIG. 16 is a graph showing the cell viability of native cells in control buffers and cells after DNA rod and nanoshell coating. Four conditions include native cells in cell media (RPMI+10% FBS), PBS and cells decorated with Rod A and the nanoshell, both in PBS. Data are presented as mean±standard deviation as indicated by error bars. n=3.

FIGS. 17A-17F show the protective effects of DNA nanoshell armor against challenging environments. FIG. 17A is fluorescence images of native cells, Rod A-coated cells, and nanoshell-coated cells after 10 minutes incubation in a range of concentrations (mass per unit volume) of sodium chloride (NaCl) of 0.9%, 0.6%, 0.3%, and 0%. Scale bars: 10 μm. FIG. 17B is a bar graph for quantifying the cell size of native cells, Rod A-coated cells, and nanoshell-coated cells under osmotic imbalanced solutions. n=100. FIG. 17C is a graph showing the relationship between cell viability and incubation in osmotic imbalanced solutions at three NaCl concentrations: 0.9%, 0.6%, and 0.3%. The representative cell images are those applied with 0.3% NaCl. FIG. 17D is a graph showing the relationship between cell viability and centrifugation. A range of centrifugation rates were tested including 110 g, 1500 g, and 3000 g. The representative cell images are those of cells applied with 3000 g. FIG. 17E is a graph showing the relationship between cell viability and fluid shear stress in a syringe-pump model. A range of flow rates were tested, which resulted in the minimal fluid shear stresses of 18, 179 and 259 dyne/cm². The representative cell images are cells applied with 259 dyne/cm². Cells in FIGS. 17C-17E were live/dead stained. Scale bars for FIGS. 17C-17E: 62.5 μm. FIG. 9F shows the multicellular assembly mediated by ssDNA and nanoshell. ssDNA-mediated assemblies were formed by a-ssDNA anchored cells (bright) and a′-ssDNA anchored cells. Nanoshell-mediated assemblies were formed by Rod A-coated cells (bright) and Rod-B coated cells. Scale bars: 25 μm. Data presented as means±s.d. as indicated by error bars (n=3). *P≤0.05, **P≤0.01,***P≤0.001.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more. A patient is a human or non-human animal.

As used herein, the term “comprising” is open-ended and may be synonymous with “including”, “containing”, or “characterized by”. As used herein, embodiments “comprising” one or more stated elements or steps also include, but are not limited to embodiments “consisting essentially of” and “consisting of” these stated elements or steps.

A “moiety” (pl. “moieties”) is a part of a chemical compound, and includes groups, such as functional groups. As such, a nucleobase moiety is a nucleobase that is modified by attachment to another compound moiety, such as a polymer monomer, e.g. the nucleic acid or nucleic acid analog monomers described herein, or a polymer, such as a nucleic acid or nucleic acid analog as described herein.

“Alkyl” refers to straight, branched chain, or cyclic hydrocarbon groups including from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. “Substituted alkyl” refers to alkyl substituted at 1 or more, e.g., 1, 2, 3, 4, 5, or even 6 positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” refer to divalent alkyl and divalent substituted alkyl, respectively, including, without limitation, ethylene (—CH₂—CH₂—). “Optionally substituted alkylene” refers to alkylene or substituted alkylene.

“Alkene or alkenyl” refers to straight, branched chain, or cyclic hydrocarbyl groups including from 2 to about 20 carbon atoms, such as, without limitation C₁₋₃, C₁₋₆, C₁₋₁₀ groups having one or more, e.g., 1, 2, 3, 4, or 5, carbon-to-carbon double bonds. “Substituted alkene” refers to alkene substituted at 1 or more, e.g., 1, 2, 3, 4, or 5 positions, which substituents are attached at any available atom to produce a stable compound, with substitution as described herein. “Optionally substituted alkene” refers to alkene or substituted alkene. Likewise, “alkenylene” refers to divalent alkene. Examples of alkenylene include without limitation, ethenylene (—CH═CH—) and all stereoisomeric and conformational isomeric forms thereof. “Substituted alkenylene” refers to divalent substituted alkene. “Optionally substituted alkenylene” refers to alkenylene or substituted alkenylene.

“Cycloalkyl” refer to monocyclic, bicyclic, tricyclic, or polycyclic, 3- to 14-membered ring systems, which are either saturated, unsaturated or aromatic. “Cycloalkenyl” refer to monocyclic, bicyclic, tricyclic, or polycyclic, 3- to 14-membered ring systems, which comprise at least one carbon-to-carbon double bond in the ring system. The cycloalkyl group may be attached via any atom. Cycloalkyl also contemplates fused rings wherein the cycloalkyl is fused to an aryl or hetroaryl ring. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with 1, 2 or 3 substituents, attached at any available atom to produce a stable compound.

As used herein a “polypeptide” includes proteins and oligopeptides as a class, and generally refers to a polypeptide comprising two or more amino acid residues, though typically referring to longer amino acid chains.

A “sample” comprising a polypeptide may be blood, plasma, serum, urine, cerebrospinal fluid, cell lysate, cell culture medium, or any other biological composition comprising a polypeptide. In one embodiment, the sample is a cell lysate from a sample of a patient's tissue. In another embodiment, the sample is prepared by lysing pelleted cells from a cell culture, e.g., to produce a recombinant protein or to analyze the proteome of cells of a cell, tissue, or organ culture.

The term “polymer composition” may be a composition comprising one or more polymers. As a class, “polymers” can include, without limitation, homopolymers, heteropolymers, co-polymers, block polymers, block co-polymers and can be both natural and synthetic. Homopolymers contain one type of building block, or monomer, whereas co-polymers contain more than one type of monomer. An “oligomer” can be a polymer that comprises a small number of monomers, such as, for example, from 3 to 100 monomer residues. As such, the term “polymer” can include oligomers. The terms “nucleic acid” and “nucleic acid analog” can include nucleic acid and nucleic acid polymers and oligomers.

A polymer “comprises” or is “derived from” a stated monomer if that monomer is incorporated into the polymer. Thus, the incorporated monomer that the polymer comprises is not the same as the monomer prior to incorporation into a polymer, in that at the very least, certain linking groups are incorporated into the polymer backbone or certain groups are removed in the polymerization process. A polymer is said to comprise a specific type of linkage if that linkage is present in the polymer. An incorporated monomer can be a “residue”. A typical monomer for a nucleic acid or nucleic acid analog is referred to as a nucleotide.

A linker is a moiety in a compound that connects one moiety to another. An “inert” linker is a moiety that covalently attaches, and optionally spaces, one moiety in a compound from another or connects a ligand or fluorophore to the backbone of a nucleic acid or nucleic acid analog and which no substantial negative effect on the activity of the overall compound, e.g., in context of the present invention, the ability of the reactive groups, such as a tetrazine group, an azide, a succidimidyl group, an alkyne containing group, or a dicarboxylic anhydride group, to react with their intended targets, and form and maintain a bond according to the methods described herein. Aside from serving to covalently-link two moieties, a linker may have a beneficial effect, such as in the physical separation of moieties to which it is attached, e.g., to optimize spacing to avoid steric effects. A linker also may serve some additional function, such as altering the hydrophobicity/hydrophilicity of the overall molecule, to provide an additional site, e.g., an amine protected by a protective group for linking additional moieties to the compound, or to rigidize the overall molecule. A linker is attached to the remainder of the compound by any suitable linkage moiety (“linkage”), e.g., by a carbon-carbon bond, an ester, a thioester, an amine, an ether, an amide, a carbonate, or a carbamate linkage to the additional moieties of the compound. Linkers typically comprise a direct bond or an atom such as oxygen, nitrogen, phosphorus, or sulfur, a unit such as, C(O), C(O)NH, SO, SO₂, SO₂NH, or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, in which one or more carbons, e.g., methylenes or methylidynes (CH═) is optionally interrupted or terminated by a hetero atom, such as O, S, or N, substituted or unsubstituted aryl, substituted or unsubstituted heteroaryl, substituted or unsubstituted heterocyclic. In one aspect, the linker may comprise or consist of between about 5 to 25 atoms, e.g., 5-20, 5-10, e.g., 5, 6, 7, 8, 9, 10, 11, 12, 13, 14, 15, 16, 17, 18, 19, or 20 atoms, or a total of from 1 to 10, e.g., 1, 2, 3, 4, 5, 6, 7, 8, 9, or 10 C and heteroatoms, e.g., O, P, N, or S atoms. The linker may have a molecular weight, based on the atomic mass of its constituent atoms, of less than 500 Daltons (Da) or less than 400 Da.

The linker may include from 1-10 carbon atoms (C₁-C₁₀), optionally substituted with a hetero-atom, such as a N, S, or O, or a non-reactive linkage, such as an amide linkage (peptide bond) formed by reacting an amine with a carboxyl group. Examples of C₁-C₁₀ alkylenes include linear or branched, alkylene (bivalent) moieties such as a methylene, ethylene, trimethylene, tetramethylene, pentamethylene, hexamethylene, hepamethylene, octamethylene, nonamethylene, or decamethylene moieties (e.g., CH₂—[CH₂]ₙ—, where n ranges from 1 to 9). The linkers may comprise from ethylene oxide (e.g., —(O—CH₂—CH₂)ₙ— or —(CH₂—CH₂—O)ₙ— (PEG) moieties, where n ranges from 2 to 100, e.g., from 2-10 (PEG₂₋₁₀), or from 2-5 (PEG₂₋₅) such as 2, 3, 4 (PEG₄), 5, 6, 7, 8, 9, or 10. A PEG linker may comprise one or more methylene groups at either end in addition to suitable linkages that attach the PEG group.

Provided herein is a ruggedized particle. In one aspect, the particle is a ruggedized live cell. The ruggedized particle comprises: a nanoparticle; and a shell comprising a plurality of first nucleic acid and/or nucleic acid analog nanostructures anchored to the particle, such as a nanoparticle; and a plurality of second nucleic acid and/or nucleic acid analog nanostructures cross-linking the first nucleic acid and/or nucleic acid analog nanostructures by a plurality of nucleic acid and/or nucleic acid analog staples. The ruggedized cell comprises: a live cell; and a shell comprising a plurality of first nucleic acid and/or nucleic acid analog nanostructures anchored to the cell; and a plurality of second nucleic acid and/or nucleic acid analog nanostructures cross-linking the first nucleic acid and/or nucleic acid analog nanostructures by a plurality of nucleic acid and/or nucleic acid analog staples

The particle may be a live cell, or a biotic or abiotic particle. Examples of particles include, without limitation: lipid-based nanoparticles or nanocarriers, such as nanoemulsions, solid lipid nanoparticles, phospholipid micelles, liposomes, vesicles, exosomes, giant unilamellar vesicles. Inorganic nanoparticles include, without limitation: iron oxide nanoparticles, gold nanoparticles, and carbon allotrope particles, such as graphene particles, nanotubes, or single-layer graphene. Polymeric nanoparticles include without limitation: PLGA (poly(lactic-co-glycolic acid)), mPEG-PGA (methoxy-poly(ethyleneglycol)-block-poly(L-glutamic acid)); PEG-PMT (PEG-poly(ω-penta-decalactone-co-N-methyldiethyleneamine sebacate-co-2,2′-thiodiethylene sebacate), and PEI-PLGA (polyethyleneimine-PLGA) particles, as merely illustrative of suitable polymeric particles. Appropriate reagents for ligand binding may be employed to link the nucleic acid and/or nucleic acid analog nanostructures to the particles, and are broadly-known in the art, including, without limitation: cholesterol-embedding in lipidic structures, antibody-receptor/antigen binding, and biotin-streptavidin binding. A non-limiting example of a biotic particle is a virus particle.

A live cell may be a multipotent cell or a pluripotent cell, such as a stem cell. For example, the cell may be a mesenchymal stem cell, a cell expressing a recombinant protein or RNA, an immune system cell, such as an antigen-presenting cell, a lymphocyte, a neutrophil, or a monocyte, or a platelet. A live cell may be eukaryotic or prokaryotic.

The first nucleic acid and/or nucleic acid analog nanostructures are anchored to the cell, such as anchored to the glycocalyx of the cell or to a surface protein of the cell (e.g., a receptor) by a click chemistry reaction, and optionally a bio-orthogonal click chemistry method.

“Click Chemistry” describes reactions that are high yielding, wide in scope, create only byproducts that can be removed without chromatography, are stereospecific, simple to perform, and can be conducted in easily removable or benign solvents. In the context of the present disclosure a click chemistry reaction is biorthogonal, meaning it is sufficiently selective that it can be performed reliably even in a complex biological environment. These reactions must proceed efficiently in the presence of the multitude of functional groups found in living systems such nucleophiles, electrophiles, reductants, oxidants, and water. Simultaneously, these reactions should have a minimal impact on the biology itself.

In the context of bio-orthogonal reactions, e.g., a bio-orthogonal click chemistry reaction, relies on bond formation between molecules or moieties not found in natural compounds, referred to herein as bio-orthogonal coupling pairs. Such reactions are preferably: selective over other potential reactive functional groups present on biomolecules, proceed in aqueous media at near physiological pH, and have fast reaction rates at room temperature (or up to 37° C.) using low reactant concentrations, all to ensure high modification efficiency (Lopes Bernardeset al. “Inverse electron demand Diels-Alder reactions in chemical biology”, Chemical Society Reviews, 2017, 46:16:4895-4950). Bio-orthogonal reaction reagents (bio-orthogonal coupling pairs) do not react with natural cellular products, such as proteins or nucleic acids. Coupling occurs under a wide range of aqueous conditions and are stable once formed.

In one example, the bio-orthogonal coupling pair is an alkyne-azide coupling pair such as a propargyl moiety or a cyclooctyne moiety and an azido moiety, as are broadly-known. The alkyne-azide coupling pair may be formed from a bio-orthogonal coupling reaction that includes an alkyne-azide reaction, e.g. with an azide group and an alkyne-containing group, such as an alkyne or alkyne-DBCO (dibenzocyclooctyne).

In these alkyne-azide bio-orthogonal coupling reactions, a compound may be provided that comprises a first member of a bio-orthogonal coupling pair, such as alkyne-containing group. For example, the first member of the bio-orthogonal coupling pair may be DBCO. The DBCO may be covalently attached, through an amide bond and an optional linker, to a single stranded DNA (ss-DNA) or a single stranded RNA (ss-RNA). The second member of the bio-orthogonal coupling pair, such as an azide ligand, may be covalently incorporated onto the cell membrane, e.g., through glycocalyx, through metabolic glycan labeling using an azido monosaccharide, e.g., N-azidoacetylmannosamine-tetraacylated (Ac4-ManNAz). The compound comprising the first member of the bio-orthogonal coupling pair, e.g. DBCO, may then be reacted with the second member of the bio-orthogonal coupling pair, e.g. the azide ligand, through a copper free click reaction to immobilize the DBCO and its covalently bound attachments (e.g., ss-DNA) (see e.g., Wijesekara et al. “Accessing and Assessing the Cell-Surface Glycocalyx Using DNA Origami”, Nano Letters, 2021, 21:4765-4773; Wang et al. “Membrane and glycocalyx tethering of DNA nanostructures for enhanced uptake”, BioRxiv, 2023).

A compound may be provided that comprises a first member of a bio-orthogonal coupling pair, such as a tetrazine moiety, linked, optionally using a linker, to a dicarboxylic anhydride moiety (see e.g., U.S. Pat. No. 11,655,271 B2). Dicarboxylic acid anhydrides, such as maleic anhydrides, e.g. citraconic anhydride, have been used in the conjugation of proteins (see, e.g., Klapper et al. “Acylation with Dicarboxylic Acid Anhydrides”, Methods Enzymol. 1972, 25:531-536; Atassi et al. “Reaction of proteins with citraconic anhydride”, Methods Enzymol., 1972, 25:546-553; Butler et al. “Maleylation of amino groups”, Methods Enzymol., 1972, 25:191-199; Kirby et al. “Structure and efficiency in intramolecular and enzymic catalysis. Catalysis of amide hydrolysis by the carboxy-group of substituted maleamic acids”, Journal of the Chemical Society Perkin Transactions, 1972, 2(9):1206-1214. The dicarboxylic anhydride moiety of the compound may react with a free primary amine (—NH₂), e.g. on a protein (see U.S. Pat. No. 11,655,271 B2) or a ssDNA, through a ring opening reaction.

The bio-orthogonal reaction may be an inverse-electron-demand Diels-Alder reaction (IEDDA, e.g., inverse electron demand [4+2] Diels-Alder cycloaddition), in which an electron-rich dienophile reacts (e.g., a strained alkene) with an electron-poor diene (e.g., a tetrazine such as a 1,2,4,5-tetrazine or a 4-(1,2,4,5-tetrazinyl)phenyl moiety such as 4-(1,2,4,5-tetrazin-3-yl)phenyl, 6-alkyl-1,2,4,5-tetrazine, 6-pyridin-2-yl-1,2,4,5-tetrazine, 6-pyrimidin-2-yl-1,2,4,5-tetrazine, 4-(6-alkyl-1,2,4,5-tetrazin-3-yl)phenyl, 4-(6-pyridin-2-yl-1,2,4,5-tetrazin-3-yl)phenyl, or 4-(6-pyrimidin-2-yl-1,2,4,5-tetrazin-3-yl)phenyl, where alkyl may be a C₁₋₄ alkyl group) (see, e.g., Karver, M R, et al., “Synthesis and Evaluation of a Series of 1,2,4,5-tetrazines for Bio-orthogonal Conjugation”, Bioconjug Chem., 2011, 22(11):2263-2270) in contrast to a normal electron demand Diels-Alder reaction, where an electron-rich diene reacts with an electron-poor dienophile (see, e.g., Lopes Bernardes, Chemical Society Reviews for further details, as well as providing examples of other bio-orthogonal reactions). Of note “1,2,4,5-tetrazine” refers to the precise 1,2,4,5-tetrazine compound or a 1,2,4,5-tetrazinyl moiety, while “a 1,2,4,5-tetrazine” refers to a compound or moiety comprising the 1,2,4,5-tetrazinyl moiety.

In IEDDA reactions, Electron-poor dienes, such as 1,2,4,5-tetrazines, are reacted with an electron-rich dienophile, for example, a strained dienophile, and fine-tuning the choice of electron-poor diene and electron-rich dienophile (“IEDDA coupling pair”) can be used to tailor the reaction kinetics (Id.). Non-limiting examples of suitable electron-poor dienes for IEDDA reactions include: tetrazines, such as 1,2,4,5 tetrazines, e.g., methyltetrazine and triazines (see, e.g., Devaraj, N K, et al., “Fast and Sensitive Pretargeted Labeling of Cancer Cells via Tetrazine/Trans-Cyclooctene Cycloaddition”, Agnew Chem Int Ed Engl., 2009, 48(38):7013-7016 and Karver, M R, et al., Bioconjug Chem., 2011, 22(11):2263-2270). A non-limiting example of an electron-rich dienophile for IEDDA reactions is trans-cyclooctene. One non-limiting example of a bio-orthogonal reaction and reaction pair is the reaction of methyl-tetrazine (mTet) and trans-cyclooctene (TCO).

As used herein, the term “nucleic acid” refers to deoxyribonucleic acids (DNA) and ribonucleic acids (RNA). Nucleic acids may comprise modified backbones, such as, for example and without limitation: 2′-O-methyl-substituted RNA, locked nucleic acids, unlocked nucleic acids, triazole-linked DNA, peptide nucleic acids including γPNA, morpholino oligomers, dideoxynucleotide oligomers, glycol nucleic acids, threose nucleic acids and combinations thereof including, but not limited to, ribonucleotide or deoxyribonucleotide residue(s). Herein, “nucleic acid” and “oligonucleotide,” in reference to nucleic acids, are used interchangeably, and can refer to a short, single-stranded structure made of up nucleotides. An oligonucleotide may be referred to by the length (i.e., number of nucleotides) of the strand, through the nomenclature “-mer”. For example, an oligonucleotide of 22 nucleotides would be referred to as a 22-mer. All nucleotide sequences are provided in a 5′ to 3′ direction, left to right, unless indicated otherwise.

A “nucleic acid analog” can be a composition comprising a sequence of nucleobases arranged on a substrate, such as a polymeric backbone, and can bind DNA and/or RNA by hybridization by Watson-Crick, or Watson-Crick-like hydrogen bond base pairing. Non-limiting examples of common nucleic acid analogs include peptide nucleic acids (PNAs), such as γPNA, nucleic acids with modified backbones, for example as described above, morpholino nucleic acids, phosphorothioates, locked nucleic acid (2′-O-4′-C-methylene bridge, including, but not limited to, oxy, thio or amino versions thereof), unlocked nucleic acid (the C2′-C3′ bond is cleaved), 2′-O-methyl-substituted RNA, threose nucleic acid, glycol nucleic acid, etc.

A conformationally preorganized nucleic acid analog can be a nucleic acid analog that has a backbone (a preorganized backbone) that forms either a right-handed helix or a left-handed helix, depending on the structure of the nucleic acid backbone. One example of a conformationally preorganized nucleic acid analog is γPNA, which has a chiral center at the γ carbon, and, depending on, and due to, the chirality of the groups at the y carbon, forms a right-handed helix or a left-handed helix. Likewise, locked nucleic acids can comprise a ribose with a bridge between the 2′ oxygen and the 4′ carbon, which “locks” the ribose into a 3′-endo (North) conformation.

In the context of the present disclosure, a “nucleotide” may refer to a monomer comprising at least one nucleobase and a backbone element (backbone moiety), which in a nucleic acid, such as RNA or DNA, is ribose or deoxyribose. “Nucleotides” also typically comprise reactive groups that permit polymerization under specific conditions. In native DNA and RNA, those reactive groups are the 5′ phosphate and 3′ hydroxyl groups. For chemical synthesis of nucleic acids and analogs thereof, the bases and backbone monomers may contain modified groups, such as blocked or protected amines. A “nucleotide residue” may refer to a single nucleotide that is incorporated into an oligonucleotide or polynucleotide. A nucleic acid or a nucleic acid analog may comprise a sequence of nucleobases, referred to herein as a binding domain, that is able to hybridize to (e.g., bind to) a complementary nucleic acid or nucleic acid analog sequence, e.g., a complementary binding domain or binding partner, on a nucleic acid by cooperative base pairing, e.g., Watson-Crick base pairing or Watson-Crick-like base pairing. Complementary binding domains may be referred to individually as binding partners, and together as a binding pair.

In further detail, nucleotides, for either RNA, DNA, or nucleic acid analogs, can have the structure A-B wherein A is a backbone monomer moiety and B is a nucleobase as described herein. The backbone monomer can be any suitable nucleic acid backbone monomer, such as a ribose triphosphate or deoxyribose triphosphate, or a monomer of a nucleic acid analog, such as peptide nucleic acid (PNA), such as a gamma PNA (γPNA). In one example the backbone monomer is a ribose mono-, di-, or tri-phosphate or a deoxyribose mono-, di-, or tri-phosphate, such as a 5′ monophosphate, diphosphate, or triphosphate of ribose or deoxyribose. The backbone monomer can include both the structural “residue” component, such as the ribose in RNA, and any active groups that are modified in linking monomers together, such as the 5′ triphosphate and 3′ hydroxyl groups of a ribonucleotide, which are modified when polymerized into RNA to leave a phosphodiester linkage. Likewise for PNA, the C-terminal carboxyl and N-terminal amine active groups of the N-(2-aminoethyl)glycine backbone monomer can be condensed during polymerization to leave a peptide (amide) bond. The active groups can be phosphoramidite groups useful for phosphoramidite oligomer synthesis. The nucleotide also optionally may comprise one or more protecting groups, such as 4,4′-dimethoxytrityl (DMT). A number of additional methods of preparing synthetic nucleic acids or nucleic acid analogs depend on the backbone structure and particular chemistry of the base addition process. Determination of which active groups to utilize in joining nucleotide monomers and which groups to protect in the bases, and the required steps in preparation of oligomers is well within the abilities of those of ordinary skill in the chemical arts and in the field of nucleic acid and nucleic acid analog oligomer synthesis.

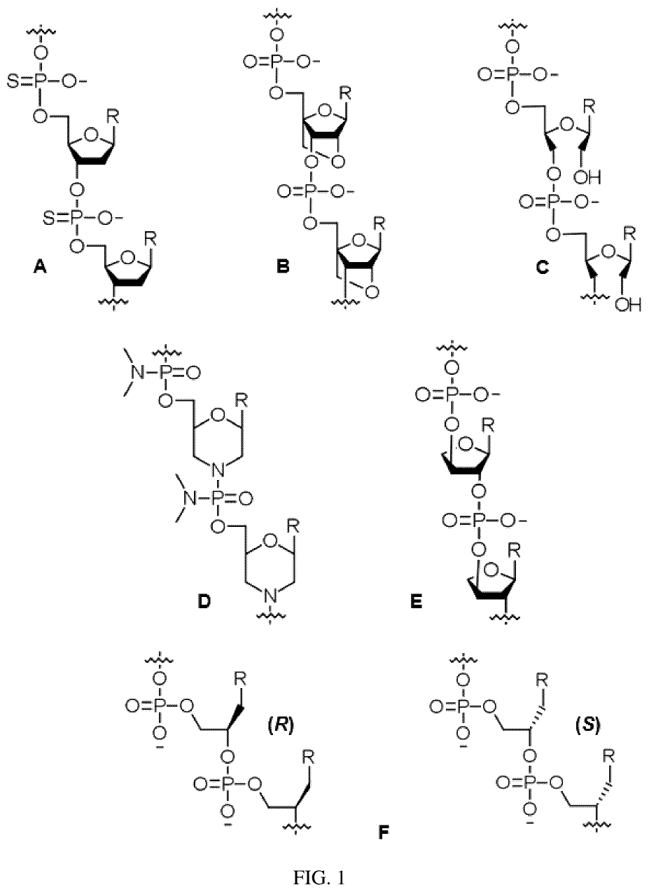

Non-limiting examples of common nucleic acid modifications for production of modified nucleic acids, e.g. nucleic acid analogs, include peptide nucleic acids, such as yPNA, phosphorothioate (e.g., FIG. 1 (A)), locked nucleic acid (2′-O-4′-C-methylene bridge, including, but not limited to, oxy, thio or amino versions thereof, (e.g., FIG. 1 (B)), unlocked nucleic acid (the C2′-C3′ bond is cleaved, e.g., FIG. 1 (C)), 2′-O-methyl-substituted RNA, morpholino nucleic acid (e.g., FIG. 1 (D)), threose nucleic acid (e.g., FIG. 1 (E)), glycol nucleic acid (e.g., FIG. 1 (F), showing R and S Forms), phosphorodiamidate morpholino oligomer (PMO). FIG. 1 (A-F) shows monomer structures for various examples of nucleic acid analogs. FIG. 1 (A-F) each show two monomer residues incorporated into a longer chain as indicated by the wavy lines. Incorporated monomers are referred to herein as “residues” and the part of the nucleic acid or nucleic acid analog excluding the nucleobases is referred to as the “backbone” of the nucleic acid or nucleic acid analog. As an example, for RNA, an exemplary nucleobase is adenine, a corresponding monomer is adenosine triphosphate, and the incorporated residue is an adenosine monophosphate residue. For RNA, the “backbone” consists of ribose subunits linked by phosphates, and, thus, the backbone monomer is ribose triphosphate prior to incorporation and a ribose monophosphate residue after incorporation. Like γPNA, Locked Nucleic Acid (e.g., FIG. 1 (B)) is conformationally preorganized. A modified nucleic acid include natural residues, e.g. natural RNA or DNA nucleosides or nucleotides, with one or more modified nucleoside or nucleotide residues. A nucleic acid analog may be a modified nucleic acid, but also includes compounds that do not contain natural RNA or DNA nucleosides, such as PNAs.

A “peptide nucleic acid” refers to a nucleic acid analog, or DNA or RNA mimic, in which the sugar phosphodiester backbone of the DNA or RNA is replaced by an N-(2-aminoethyl)glycine unit. A gamma PNA (γPNA) is an oligomer or polymer of gamma-modified N-(2-aminoethyl)glycine monomers of the following structure:

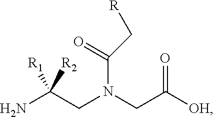

where R is a nucleobase moiety, and at least one of R₁ or R₂ attached to the gamma carbon is not a hydrogen, such that the gamma carbon is a chiral center. When R₁ and R₂ are hydrogen (N-(2-aminoethyl)-glycine backbone), or the same, there is no such chirality about the gamma carbon. Where R₂ is hydrogen, and R₁ is not, the γPNA is said to be left-handed. Where R₁ is hydrogen, and R₂ is not, the γPNA is said to be right-handed. Right-handed PNA is able to hybridize in a Watson-Crick or Watson-Crick-like manner with complementary single-stranded DNA or RNA. In some cases where neither R₁ nor R₂ are hydrogen but R₁ and R₂ are different, the handedness of the residue or oligomer may be dictated by the respective bulkiness of the R₁ and R₂ groups, or by other physical or chemical properties of the groups, and the chirality may be designated right-handed where the configuration hybridizes to complementary DNA or RNA strands, but the opposite configuration does not.

R₁ and R₂ may include, but are not limited to, any one of the following function groups:
(1) dAmino acid sidechains (Ala, —CH₃; Val, —CH(CH₃)₂; Ile, —CH(CH₃)CH₂CH₃; Leu, —CH₂CH(CH₃)₂; Met, —CH₂CH₂SCH₃; Phe, —CH₂C₆H₅; Tyr, —CH₂C₆H₄OH; Trp, —CH₂C₈l-₁₅NH; Ser, —CH₂OH; HSer, —CH₂CH₂OH; Thr, —HCH₃OH; Asn, —CH₂CONH₂; Gln, —CH₂CH₂CONH₂; Cys, —CHSH; Sec, —CH₂SeH; Gly, —H; Pro, —(CH₂)3—; Arg, —(CH₂)₃NHC (NH)NH₂; His, —CH₂C₃H₃N₂; Lys, —(CH₂)₄NH₂; Asp, —CH₂CO₂H; and Glu, —(CH₂)₂CO₂H).
(2) Linear or branched (C₁-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene, (C₃-C₈)cycloalkyl(C₁-C₆)alkylene, —CH₂(OCH₂—CH₂)_{q}OH, —CH₂—(OCH₂—CH₂)_{q}—NH₂, —CH₂—(OCH₂—CH₂)_{q}—NHC(NH)NH₂, —CH₂—(OCH₂—H₂—O)_{q}—SH and —CH₂—SCH₂—CH₂)_{q}—SH, —(CH₂CH₂)_{q}—NHC(NH)NH₂, where subscript q is an integer between 0-25.

An incorporated PNA or γPNA monomer,

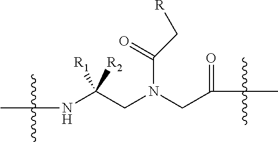

is referred to herein as a PNA or γPNA “residue”, in reference to the remaining structure after integration into an oligomer or polymer, with each residue having the same or different R group as its base (nucleobase), such as adenine, guanine, cytosine, thymine and uracil bases, or other bases, such as the monovalent and divalent bases described herein, such that the order of bases on the PNA is its “sequence”, as with DNA or RNA. A sequence of nucleobases in a nucleic acid or a nucleic acid analog oligomer or polymer, such as a PNA or yPNA oligomer or polymers, binds to a complementary sequence of adenine, guanine, cytosine, thymine and/or uracil residues in a nucleic acid or nucleic acid analog strand by nucleobase pairing, in a Watson-Crick or Watson-Crick-like manner, essentially as with double-stranded DNA or RNA.

A “guanidine” or “guanidinium” group may be added to the recognition reagent to increase solubility and/or bioavailability. Because PNA is produced in a similar manner to synthetic peptides, a simple way to add guanidine groups is to add one or more terminal arginine (Arg) residues to the N-terminal and/or C-terminal ends of the PNA, e.g., γPNA, recognition reagent. Likewise, an arginine side group,

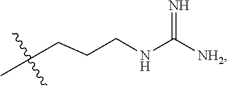 [SMILES: *CCCNC(=N)N]

or a guanidine-containing moiety, such as

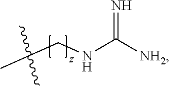

where z, for example and without limitation, ranges from 1-5, or a salt thereof, can be attached to a recognition reagent backbone as described herein. A guanidine-containing group is a group comprising a guanidine moiety, and may have less than 100 atoms, less than 50 atoms, e.g., less than 30 atoms. In one aspect, the guanidine-containing group has the structure:

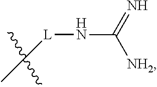

where L is a non-reactive linker, e.g., a non-reactive aliphatic hydrocarbyl linker, such as a methylene, ethylene, trimethylene, tetramethylene, or pentamethylene linker. The guanidine-containing group may have the structure:

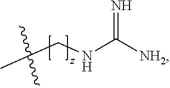

where z is 1-5, e.g., the guanidine group may be an arginine side group.

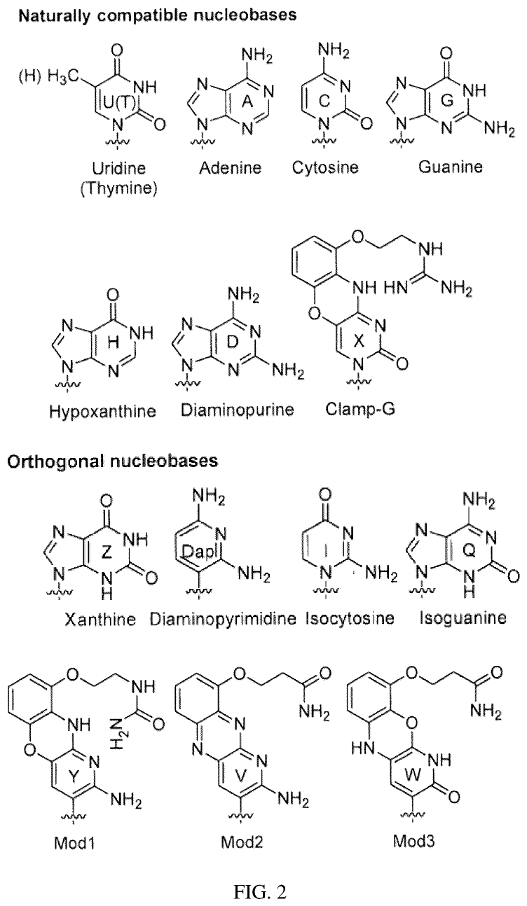

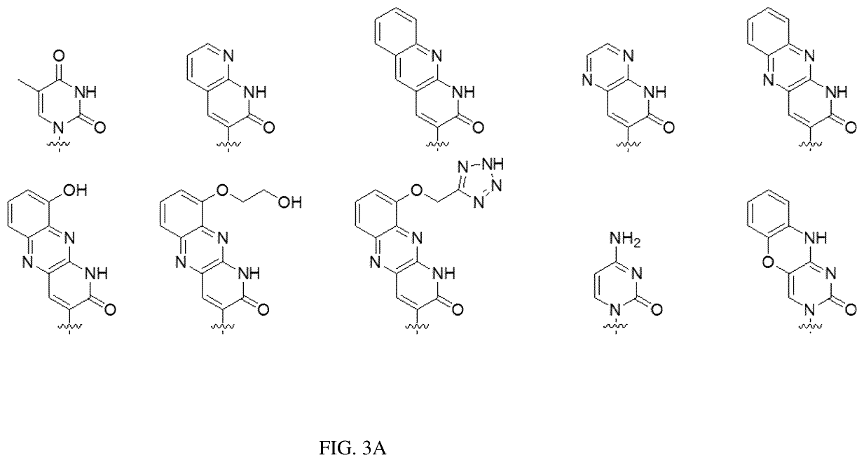

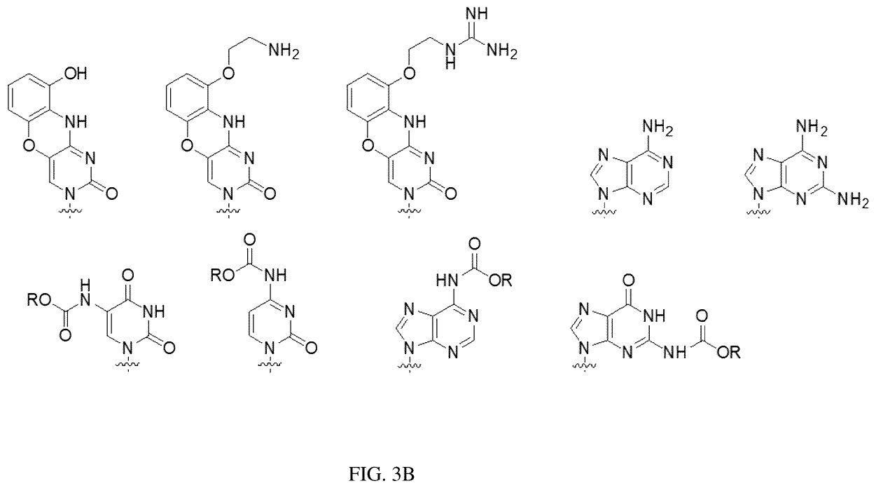

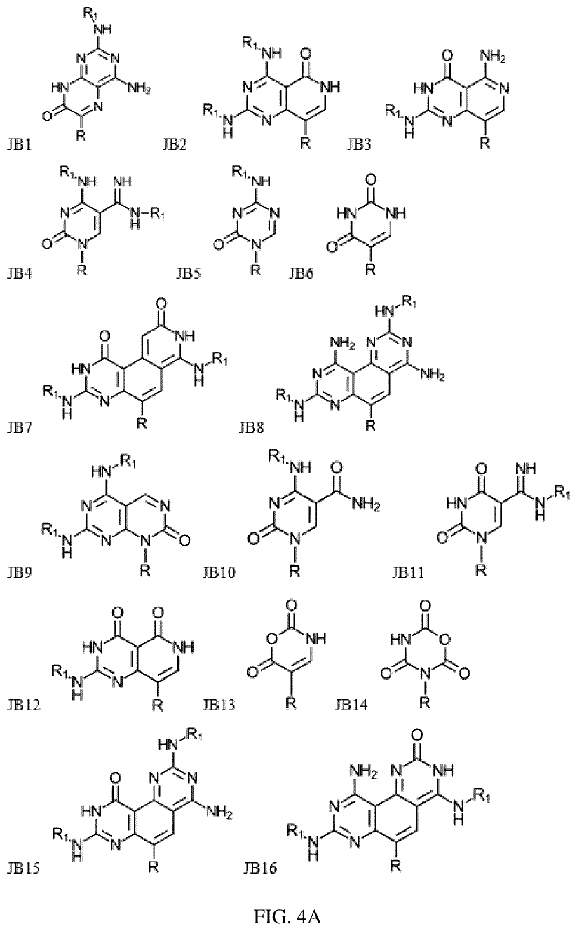

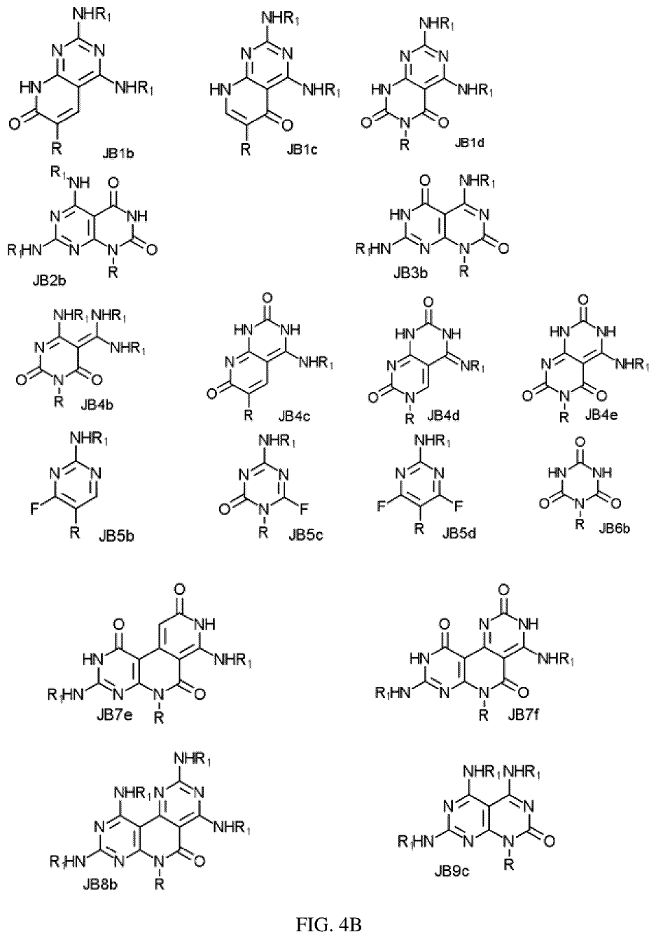

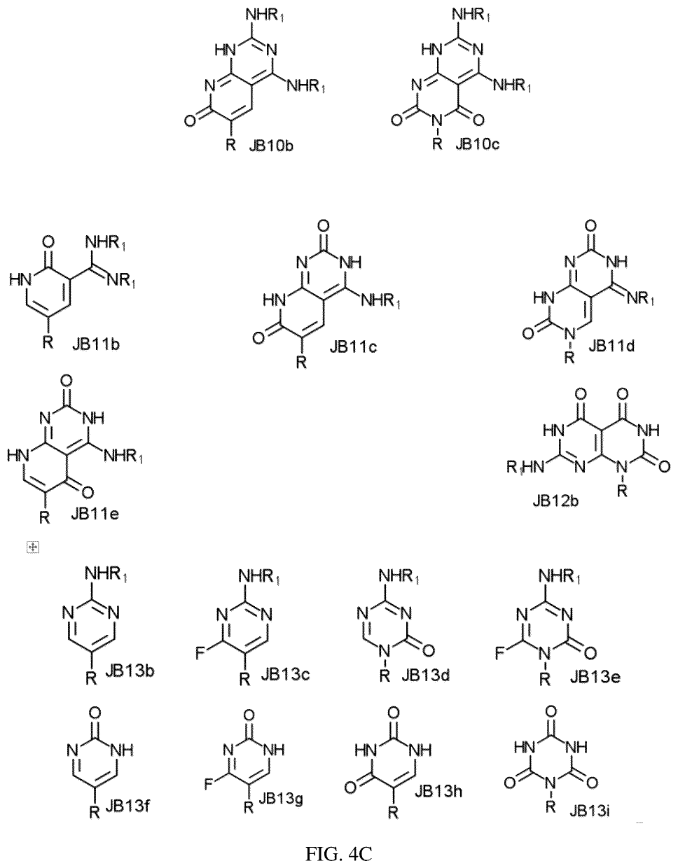

A “nucleobase” can include primary nucleobases: adenine, guanine, thymine, cytosine, and uracil, as well as modified, non-natural, purine and pyrimidine bases, such as, without limitation, hypoxanthine, xanthene, 7-methylguanine, 5, 6, dihydrouracil, 5-methylcytosine, and 5-hydroxymethylcytosine. FIGS. 2, 3A, 3B, and 4A-4C also depict non-limiting examples of nucleobases, including, but not limited to, monovalent nucleobases (e.g., adenine, cytosine, guanine, thymine or uracil, which bind to one strand of nucleic acid or nucleic acid analogs), and divalent nucleobases (e.g., JB1-JB16 described herein) which bind complementary nucleobases on two strands of DNA simultaneously, and “clamp” nucleobases, such as a “G-clamp,” which binds complementary nucleobases with enhanced strength. Additional purine, purine-like, pyrimidine and pyrimidine-like nucleobases are disclosed, for example in United States Pat. Nos. 8,053,212, 8,389,703, and 8,653,254. For divalent nucleobases JB1-JB16, shown in FIG. 4A, Table A shows the specificity of the different nucleobases. Of note, JB1-JB4 series bind complementary bases (C-G, G-C, A-T and T-A), while JB5-JB16 bind mismatches, and, thus, can be used to bind two strands of matched and/or mismatched bases. Divalent nucleobases are described in further detail in United States Patent Application Publication No. 2016/0083434 A1 and International Patent Publication No. WO 2018/058091, both of which are incorporated herein by reference.

TABLE A
Divalent Nucleobases

| Nucleobase | Bases represented |
|---|---|
| JB1 | T/D* |
| JB2 | D/T |
| JB3 | G/C |
| JB4 | C/G |
| JB5 | C/C |
| JB6 | U/U |
| JB7 | G/G |
| JB8 | D/D |
| JB9/JB9b | A/C |
| JB10 | C/A |
| JB11 | U/G |
| JB12 | G/U |
| JB13 | C/U |
| JB14 | U/C |
| JB15 | G/D |
| JB16 | D/G |

*diaminopurine, an adenine analog.

Exemplary γPNA structures are disclosed in International Patent Publication No. WO 2012/138955, incorporated herein by reference. The γPNA may be left-handed PNA, which is orthogonal to natural RNA and DNA.

Complementary refers to the ability of polynucleotides (nucleic acids and nucleic acid analogs) to hybridize (anneal) to one another, forming inter-strand base pairs. Annealing is temperature-dependent, and complementary strands anneal at a temperature below the melting temperature (Tm) for the strands, which depends on, for example, the respective nucleobase sequences of the strands, the length of the complementary sequences, the solvent in which the strands are dissolved, and the backbone structure of the strands. Base pairs are formed by hydrogen bonding between nucleotide units in polynucleotide or polynucleotide analog strands that are typically in antiparallel orientation. Complementary polynucleotide strands can base pair (hybridize) in the Watson-Crick manner (e.g., A to T, A to U, C to G), or in any other manner that allows for the formation of duplexes. In RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline) or phosphate-buffered saline, in a polar aprotic solvent or polar aprotic organic solvent, such as DMSO and/or DMF, optionally in the presence of an anionic surfactant, as described herein, or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, e.g., by salt concentration and temperature, with the melting temperature (Tm) lowering with increased mismatches and increased stringency. Perfectly matched sequences are said to be “fully complementary”, though one sequence (e.g., a target sequence in an mRNA) may be longer than the other.

A moiety in a compound, such as a fluorophore or ligand such as biotin, can be covalently attached to the nucleic acid or nucleic acid analog backbone, and, thus, is said to be “linked” to the backbone. Depending on the chemistry used to prepare the compound, the linkage may be direct, or through a “linker” which is a moiety that covalently attaches two other moieties or groups. The linkers are non-bulky in that they do not sterically hinder or otherwise interfere to any substantial extent with the formation of a DNA origami structure. The linker, when incorporated into a compound is the remaining moiety or residue resulting from the linking. It is noted in the context of nucleic acid nanostructures as described herein, single-stranded nucleic acids, such as ssDNA, can serve as a linker.

Environmental sensors, such as force sensors, may be built into or linked to the nucleic acid and/or nucleic acid analog nanostructures. Beltran SM et al. (Beltran S M, Slepian M J, Taylor R E. Extending the Capabilities of Molecular Force Sensors via DNA Nanotechnology. Crit Rev Biomed Eng. 2020;48(1)1 -16) describe exemplary molecular tension sensors. Fluorophore Förster Resonance Energy Transfer (FRET) pairs are sensitive to separation and relative rotation. DNA duplexes can unzip or flex in response to load, thereby sensing force quantitatively. In this way attached molecules can sense solution environment conditions and respond fluorescently, plasmonically, electrically, etc. Non-limiting examples of micromechanical force sensors are disclosed in U.S. Pat. No. 11,708,601, the disclosure of which is hereby incorporated herein by reference in its entirety.

In one example, a spring force sensor may be attached to a nucleic acid and/or nucleic acid analog nanostructure linked to a cell as described herein. The micromechanical spring sensor may comprise:
a spring element comprising a nucleic acid and/or nucleic acid analog nanostructure beam pair comprising one or more single-stranded scaffold nucleic acids or nucleic acid analogs, and nucleic acids or nucleic acid analog staples, comprising:
a flexible first beam and a flexible second beam pinned together at two or more spaced-apart pinning locations on the beams and defining unpinned sections of the beams and beam pair between the pinning locations, the beam pair comprising two or more fluorescent donor moieties arranged in the unpinned section of the first beam and two or more quenching acceptor moieties that quench the signal of the donor and which are spatially aligned in the unpinned section of the second beam with a corresponding donor moiety on the first beam, defining a gap distance between the corresponding donor and acceptor moieties;
a first tether attached to the first beam at the unpinned section; and
a second tether attached to the second beam at the unpinned section;
wherein the unpinned section of the beams flex from a closed position with no tensional force applied to the tethers, defining a resting gap distance between the fluorescent donor moieties and the quenching acceptor moieties, to an open position with the application of a tensional force to the tethers where the beams flex apart to an open position with the first and second beam remaining pinned together at the pinning locations, defining a tensioned gap distance, wherein the resting gap distance between the fluorescent donor moieties and the quenching acceptor moieties is less than the tensioned gap distance between the fluorescent donor moieties and the quenching acceptor moieties for at least one pair of corresponding donor and acceptor moieties, such that the sensor produces a detectably different emission spectra when the beams are in the closed position as compared to when the beams are in the open position; and
a drag element attached to a tether of one of the beam pairs. Fluorescence and quenching thereof is one manner in which forces acting on a cell may be determined. Other environmental sensor pair types and detection methods may be used to detect forces acting on the ruggedized cell as disclosed herein.

Fluorescent or fluorescence refers to the ability of a fluorophore to absorb electromagnetic radiation, for example ultraviolet radiation or light, to reach an excited state, and release a photon upon relaxation to a lower energy state, (e.g., a ground state). The excited state may be metastable, resulting in phosphorescence. Transfer of the energy of the fluorophore to another molecule is referred to as quenching, examples of which include Förster Resonance Energy Transfer (FRET), along with other mechanisms.

A FRET pair is a pair of chromophores, e.g., fluorophores, that, when placed within a sufficiently small radius from each-other, undergo resonance energy transfer from a first member of the FRET pair (donor) to the second member of the FRET pair (acceptor or quenching acceptor). The process of resonance energy transfer takes place when a donor fluorophore in an electronically excited state transfers its excitation energy to a nearby chromophore, the acceptor, such that the acceptor quenches the donor. The emission spectrum for the donor may overlap with the excitation spectrum for the acceptor. When the donor and acceptor in a FRET pair are sufficiently close together, excitation of the donor results in the transfer of the excitation energy to the acceptor (quenching of the donor by the acceptor), resulting in fluorescence at the emission wavelength of the acceptor. As the distance between the donor and acceptor in a FRET pair increases, excitation of the donor results in increasingly lesser transfer of the excitation energy to the acceptor, resulting in lower fluorescence, or no fluorescence at the emission wavelength of the acceptor, and typically increased emission at the emission wavelength of the donor.

Primary criteria for FRET to occur can be as follows: donor and acceptor molecules are in close proximity (typically 10-100 A); the absorption spectrum of the acceptor overlaps the fluorescence emission spectrum of the donor; and donor and acceptor transition dipole orientations are approximately parallel.

The Förster critical distance (R(0)) is the acceptor-donor separation distance for which the transfer rate equals the rate of donor decay (de-excitation) in the absence of acceptor. In other words, when the donor and acceptor radius (r) equals the Förster critical distance, then the transfer efficiency is 50 percent. At this separation radius, half of the donor excitation energy is transferred to the acceptor via resonance energy transfer, while the other half is dissipated through a combination of all the other available processes, including fluorescence emission of the donor. The Förster critical distance value typically falls within a range of 2 to 6 nanometers. Roy et al. (Roy et al. “A practical guide to single-molecule FRET.” Nature methods vol. 5(6) (2008): 507-16. doi:10.1038/nmeth.1208) among many other resources describe the FRET process and options for FRET pairs.

A large variety of small molecule fluorophores (e.g., 2000 Da or less, or 1000 Da or less, such as cyanine, xanthine, squaraine, among many other dyes, see, e.g., FPbase (fpbase.org) and fluorophores.tugraz.at) or protein fluorophores (typically 10 kDa or larger) are known and are shown to be effective in FRET pairs, and can be arranged on the beams of the spring sensor described herein to produce FRET activity when the beam is in its closed configuration, and to produced progressively less FRET activity on application of tensional force and flexing the beams of the sensor to an open configuration. Selection of FRET pairs may be made by determining the presence of overlap in the emission spectra of the donor and excitation spectrum of the acceptor (see, e.g., Roy et al., providing non-limiting examples of certain small molecule FRET donors, such as Cy3, ATT0550, and Alexa555, and certain small molecule FRET acceptors, such as Cy5, ATTO647N, and Alexa647, and describing methods of conjugating the dyes or biotin to nucleic acids (see, Table 2 of Roy et al.).

Example of protein FRET pairs include, without limitation: ECFP-EYFP; mTurquoise2-sEYFP; mTurquoise2-mVenus; EGFP-mCherry; Clover-m Ruby2; mClover3-mRuby3; mNeonGreen-mRuby3; eqFP650-iRFP; mAmetrine-tdTomato; LSSmOrange-mKate2; EGFP-sREACh; EGFP-ShadowG; EGFP-activated PA-GFP; EGFP-Phanta; mTagBFP-sfGFP; mVenus-mKOK; and CyOFP1-mCardinal (See, e.g., Bajar B T, et al. A Guide to Fluorescent Protein FRET Pairs. Sensors (Basel). 2016;16(9):1488).

The quenching acceptor can be a fluorescent quencher that fluoresces at its emission spectrum upon excitation by the donor. The quenching acceptor can be a dark quencher, which is non-fluorescent, but may dissipate energy received from the donor by non-fluorescent means, such as by heat dissipation (e.g., by molecular vibration). That is, the excited quencher acceptor may return to the ground state through fluorescent emission or nonradiatively (dark quenching). Non-limiting examples of dark quenchers include: Dabsyl (dimethylaminoazobenzenesulfonic acid), BLACK HOLE QUENCHER® Dyes, QXL™ quenchers, IOWA BLACK® FQ, IOWA BLACK® RQ, and IRDye® QC-1, among others.

Additional types of environmental sensors may be attached to the shell as described herein, such as, for example and without limitation: pH sensors and redox sensors.

In one example, the nucleic acid or nucleic acid analog may be peptide-nucleic acid, such as gamma-PNA (γPNA). For linkage to a nucleic acid or nucleic acid analog, any expedient and available linker may be one that reacts with a hydroxyl or phosphate group, and in the case of PNA, with an amine or carboxyl group to form an amide linkage, e.g., using peptide synthesis chemistries to add amino acids to the recognition reagent, where amino acids, such as arginine, may be pre-modified with a chemical moiety, such as a left-handed γPNA, or a guanidine group. Linking to non-peptide nucleic acid analogs can be achieved using any suitable linking chemistry, such as by using carbodiimide chemistry.

A single-stranded gamma peptide nucleic acid (ss-γPNA) may be included in the nanostructure. The composition may comprise a PNA backbone, and thus has a structure exemplified by:

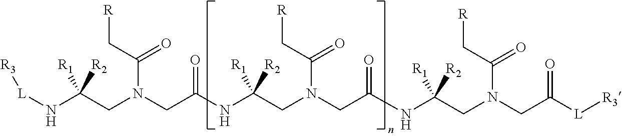

where, each R is independently, nucleobases;
n is an integer ranging from 1 and 6, such as 1, 2, 3, 4, 5, or 6;
each L is independently, linkers;
each R₁ and R₂ may be, independently: a guanidine-containing group such as

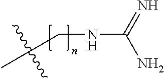

where n=1, 2, 3, 4, or 5; an amino acid side chain, such as:

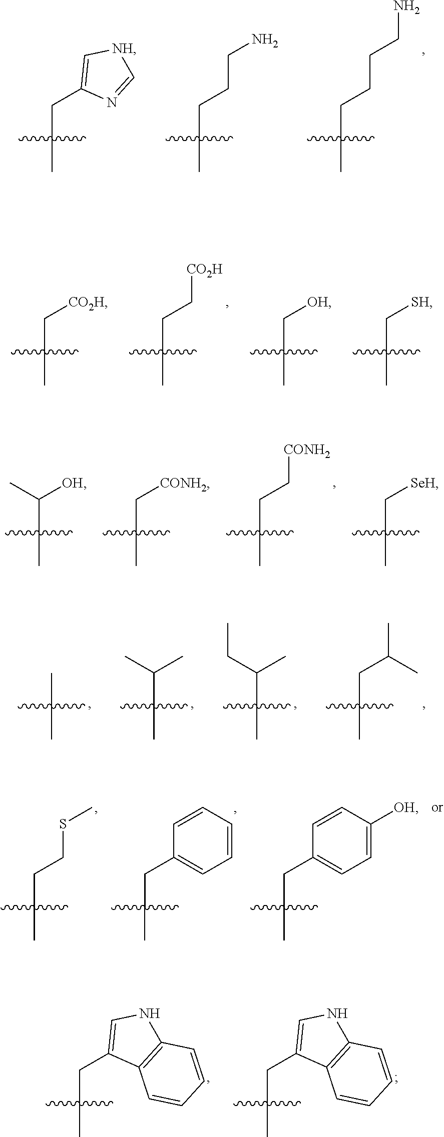

methyl, ethyl, linear or branched (C₃-C₈)alkyl, (C₂-C₈)alkenyl, (C₂-C₈)alkynyl, (C₁-C₈)hydroxyalkyl, (C₃-C₈)aryl, (C₃-C₈)cycloalkyl, (C₃-C₈)aryl(C₁C₆)alkylene, (C₃-C₈)cycloalkyl(C₁-C₆)alkylene, each optionally substituted with a polyethylene glycol chain of 1 to 50 units; H, —CH₂—(OCH₂—CH₂)_{q}OP₁; CH₂—(OCH₂-CH₂)_{q}—NHP₁; —CH₂—(SCH₂—CH₂)_{q}—SP₁; —CH₂—(OCH₂—CH₂)ᵣ—OH; —CH₂—(OCH₂—CH₂)ᵣ—NH₂; —CH₂—(OCH₂—CH₂)ᵣ—NHC(NH)NH₂; or —CH₂—(OCH₂—CH₂)ᵣ—S—S[CH₂CH₂]ₛNHC(NH)NH₂, where P₁ is H, (C₁-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene or (C₃-C₈)cycloalkyl(C₁-C₆)alkylene; q is an integer from 0 to 50; r is an integer from 1 to 50, and s is an integer from 1 to 50; where, for γPNA R₁ and R₂ are different, R₁ is H and R₂ is not H, R₂ is H and R₁ is not H, or R₂ is not H and R₁ is not H but is different than R₂ so as to form a chiral center at the γ carbon. For binding to natural nucleic acids, such as RNA or DNA, R₁ is H and R₂ is not H, thereby forming “right-handed” L-γPNA. “Left-handed” D-γPNA, in which R₂ is H and R₁ is not H, does not bind natural nucleic acids; and
R₃ and R₃′ are terminal groups, such as H, a guanidine-containing group, a tag, a dye or fluorophore, one or more consecutive amino acids, such as arginine or lysine, or a detectable or capturable moiety, such as, for example and without limitation, a cyanine dye, a his tag, or biotin.

Each instance of L is, independently, a linker, and may comprise one or more amino acid residues, or substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, in which one or more carbons, e.g., methylenes or methylidynes (—CH═) is optionally interrupted or terminated by a hetero atom, such as O, S, or N, substituted or unsubstituted aryl, substituted or unsubstituted heteroaryl, substituted or unsubstituted heterocyclic, and optionally comprises a guanidine-containing group such as

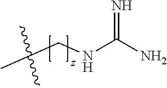

where z=1, 2, 3, 4, or 5, and/or an amino acid side chain.

Each L is optional and may comprise or consist of between about 5 to 25 atoms, e.g., 5-20, 5-10, e.g., 5, 6, 7, 8, 9, 10, 11, 12, 13, 14, 15, 16, 17, 18, 19, or 20 atoms, or a total of from 1 to 10, e.g., 1, 2, 3, 4, 5, 6, 7, 8, 9, or 10 C and heteroatoms, e.g., O, P, N, or S atoms. In one aspect, R₁ or R₂ may be (C₁-C₆)alkyl substituted with —(OCH₂—CH₂)_{q}OP₁; —(OCH₂—CH₂)_{q}—NHP₁; —(SCH₂—CH₂)_{q}—SP₁; —(OCH₂—CH₂)ᵣ—OH; —(OCH₂—CH₂)ᵣ—NH₂; —(OCH₂) CH₂)ᵣ—NHC(NH)NH₂; or —(OCH₂—CH₂)ᵣ—S—S[CH₂CH_{2]}NHC(NH)NH₂, where P₁ is H, (C₁-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene or (C₃-C₈)cycloalkyl(C₁-C₆)alkylene; q is an integer from 0 to 50; r is an integer from 1 to 50, and s is an integer from 1 to 50.

Unless otherwise indicated, the nucleic acids and nucleic acid analogs described herein are not described with respect to any particular sequence of nucleobases. The present disclosure is directed to ruggedized particles, such as ruggedized cells comprising a live cell and a shell and methods in preparing the ruggedized particles or cells and compositions for use, and is independent of the identity and sequence of bases attached thereto. Any nucleobase sequence attached to the backbone of the described oligomers can hybridize in a specific manner with a complementary nucleobases sequence of a target nucleic acid or nucleic acid analog by Watson-Crick or Watson-Crick-like hydrogen bonding. The compositions and methods described herein are sequence-independent and describe a novel, generalized method, and related compositions, for ruggedized particle or cell production.

Nucleobases of the binding domains described herein are arranged in a sequence complementary to target sequences of other binding domains, so that binding domains as described herein bind by base pairing, e.g., by Watson-Crick, or Watson-Crick-like base pairing.

As above, the ruggedized particles or cells of the present application comprises a particle or live cell; and a shell comprising a plurality of first nucleic acid and/or nucleic acid analog nanostructures anchored to the cell; and a plurality of second nucleic acid and/or nucleic acid analog nanostructures cross-linking the first nucleic acid and/or nucleic acid analog nanostructures by a plurality of nucleic acid and/or nucleic acid analog staples.

The structures, such as beams, rods, helices, balls, or sheets, described herein, comprise ordered nucleic acid or nucleic acid analogs, and are referred to herein as nucleic acid and/or nucleic acid analog nanostructure (e.g., DNA nanostructure), produced from nucleic acids (including modified nucleic acids) and/or nucleic acid analogs. Nucleic acid and/or nucleic acid analog nanostructures may be nucleic acid and/or nucleic acid analog origami (e.g., DNA origami) structures or tiled structures.

For example, a ruggedized particle or cell may comprise the particle or a live cell; a plurality of first nucleic acid and/or nucleic acid analog nanostructure beams anchored to the particle or cell, the beams comprising one or more single-stranded scaffold nucleic acids or nucleic acid analogs; nucleic acids and/or nucleic acid analog staples attached to the plurality of first nucleic acid and/or nucleic acid analog nanostructure beams and nucleic acids and/or nucleic acid analog staples attached to the plurality of second nucleic acid and/or nucleic acid analog nanostructures, wherein the nucleic acids and/or nucleic acid analog staples attached to the plurality of second nucleic acid and/or nucleic acid analog nanostructures are complementary to the nucleic acids and/or nucleic acid analog staples attached to the plurality of first nucleic acid and/or nucleic acid analog nanostructure beams; and the plurality of second nucleic acid and/or nucleic acid analog nanostructure linked to the plurality of first nucleic acid and/or nucleic acid analog nanostructure beams by the staples and which cross-link two or more of the first nucleic acid and/or nucleic acid analog nanostructures to the second nucleic acid and/or nucleic acid analog nanostructures. The plurality of second nucleic acid and/or nucleic acid analog nanostructures may be second nucleic acid and/or nucleic acid analog nanostructure beams. Alternatively, the plurality of second nucleic acid and/or nucleic acid analog nanostructures may have a structure that is different from the plurality of plurality of first nucleic acid and/or nucleic acid analog nanostructure beams (i.e., the plurality of second nucleic acid and/or nucleic acid analog nanostructures are not beams).

“DNA origami” and “nucleic acid and/or nucleic acid analog origami” generally refers to the folding of a large, single-stranded nucleic acid (the “scaffold”) by annealing with smaller “staple” nucleic acids to form a designated structure. Staples may be fully incorporated within the nucleic acid and/or nucleic acid analog structure, or may comprise a pendant portion that does not bind to the scaffold or to a staple and which extends (pends) from the structure. The pendant portions may be linked to a drug molecule, e.g., via a labile bond, such as an ester linkage, or linked to (labeled with) a tag, such as a fluorophore, a fluorescent protein, or a quenching acceptor moiety. The pendent portions may be used as a “tether” to attach other structures or compounds to the structure either by annealing of complementary nucleic acid strands present on other structures or an intermediate linking compound or structure, or by covalent linkage of a member of a binding pair to the tether. For example, a tether may be biotinylated to bind with avidin/streptavidin. A tether may include an aptamer sequence for specific binding to a target molecule. A tether may be linked to an antibody or a fragment thereof, e.g., a paratope, for specific binding to a target antigen or epitope. A pinning staple is a type of staple used to connect two larger nucleic acid and/or nucleic acid analog origami structures, such as the beams described herein, and may comprise a pendant portion used to anneal with a pendant portion of another structural element, such as another beam, a plate, or spherical/ball structure, as described herein.

The plurality of first nucleic acid and/or nucleic acid analog nanostructures and/or the plurality of second nucleic acid and/or nucleic acid analog nanostructures may comprise at least 1, such as from 3 to 30 nucleic acid and/or nucleic acid analog staples, such as 3, 4, 5, 6, 7, 8, 9, 10, 11, 12, 13, 14, 15, 16, 17, 18, 19, 20, 21, 22, 23, 24, 25, 26, 27, 28, 29, or 30 staples. For example, the nucleic acid and/or nucleic acid analog staples may be ss-DNA having five or more bases in the sequence. The nucleic acid and/or nucleic acid analog staples may be positioned at the edge of the first nucleic acid and/or nucleic acid analog nanostructures and/or the edge of the second first nucleic acid and/or nucleic acid analog nanostructures. Alternatively, the nucleic acid and/or nucleic acid analog staples may be positioned at the center of the first nucleic acid and/or nucleic acid analog nanostructures and/or the center of the second first nucleic acid and/or nucleic acid analog nanostructures.

The first nucleic acid and/or nucleic acid analog nanostructures and/or the second first nucleic acid and/or nucleic acid analog nanostructures may comprise nucleic acid and/or nucleic acid analog nanostructure beams. A beam may be a single rod nucleic acid nanostructure, or may be a larger bundle of rods, e.g. a tube, formed from a bundle of single rods, such as from 2-20 rods (e.g., helical nucleic acid and nucleic acid analog bundles), e.g. 2, 3, 4, 5, 6, 7, 8, 9, 10, 11, 12, 13, 14, 15, 16, 17, 18, 19, or 20 rods, or even from more rods. The rods in a bundle of rods may include multiples of the same rod, or may be formed from different rods.

For example, the first nucleic acid and/or nucleic acid analog nanostructure may be a beam comprising from two to ten rods (e.g., helical nucleic acid and/or nucleic acid analog bundles), such as six rods and the second nucleic acid. For example, the second nucleic acid and/or nucleic acid analog nanostructure may be a beam comprising from two to ten rods (e.g., helical nucleic acid and/or nucleic acid analog bundles), such as six rods. The beams of the first nucleic acid and/or nucleic acid analog nanostructure may comprise from 3 to 30 nucleic acid and/or nucleic acid analog staples and the nucleic acid and/or nucleic acid analog staples may be positioned in the center. The beams of the second nucleic acid and/or nucleic acid analog nanostructure may comprise from 3 to 30 nucleic acid and/or nucleic acid analog staples and the nucleic acid and/or nucleic acid analog staples may be positioned in the center.

The beams may comprise a diameter of at least 3 nanometers (nm), such as from 3 nm to 10 nm, from 4 nm to 9 nm, from 5 nm to 8 nm, or from 6 nm to 8 nm. The beam may comprise a length of at least 200 nanometers and not greater than 100 microns (μm), such as from 200 nm to 90 μm, from 200 nm to 50 μm, from 200 nm to 10 pm, from 200 nm to 1 pm, from 200 nm to 900 nm, from 200 nm to 800 nm, from 200 nm to 700 nm, from 200 nm to 600 nm, from 200 nm to 600 nm, from 200 nm to 500 nm, from 300 nm to 700 nm, from 300 nm to 600 nm, or from 300 nm to 500 nm. A cell membrane anchor is a chemical moiety that comprise one or more hydrophobic or lipophilic moieties that intercalate, traverse, or otherwise integrate into a cell's lipid bilayer. A cell membrane anchor may be attached to a first nucleic acid and/or nucleic acid analog nanostructure as described herein such that the first nucleic acid and/or nucleic acid analog nanostructure may be anchored to a cell membrane of a cell to be incorporated into a ruggedized cell structure as described herein. Anchoring to a cell membrane is in contrast to anchoring to an already-anchored cell surface protein or glycocalyx structure. Many, if not most eukaryotic and prokaryotic cells are covered by a dense layer of carbohydrate moieties of glycoproteins and glycolipids, collectively termed the “glycocalyx”. In animal cells, the glycocalyx is an organelle of vital significance, actively involved in and functionally relevant for various cellular processes. The glycocalyx can be involved in, for example, cell recognition, cell communication, intercellular adhesion, and barrier functions (see e.g., Möckl, L. “The Emerging Role of the Mammalian Glycocalyx in Functional Membrane Organization and Immune System Regulation”, Front Cell Dev Biol., 2020, 15(8):253). A cell membrane anchor moiety may comprise a protein or polypeptide, such as a transmembrane segment of a membrane-anchored protein. A cell membrane anchor moiety may comprise a non-peptidyl hydrophobic moiety, which typically comprise one or more fatty acid moieties. A classic example of a hydrophobic moiety that can be attached to the first nucleic acid and/or nucleic acid analog nanostructure to anchor it into a cell membrane is a glycosylphosphatidylinositol (GPI) anchor (see, e.g., Paulick M G, Bertozzi C R. The glycosylphosphatidylinositol anchor: a complex membrane-anchoring structure for proteins. Biochemistry. 2008 Jul. 8;47(27):6991-7000) such as phosphatidylinositol. Other fatty acid-containing, or cholesterol-containing moieties, including phospholipids, glycerophospholipids, phosphatidylcholine, phosphatidylethanolamine, and similar amphiphilic structures may be utilized, and are well-known in the art and are commercially available. Any of these may be linked to a nucleic acid and/or nucleic acid analog nanostructure by conventional methods (see e.g., Liu et al. “The effects of overhang placement and multivalency on cell labeling by DNA origami”, Nanoscale, 2021, 13:6819-6828).

In one non-limiting embodiment, the plurality of first nucleic acid and/or nucleic acid analog nanostructures are anchored to the glycocalyx of the cell. In another non-limiting embodiment, the plurality of first nucleic acid and/or nucleic acid analog nanostructures are anchored to a surface protein of the cell, such as a receptor.

The plurality of first nucleic acid and/or nucleic acid analog nanostructures may form a first layer about the cell, and the plurality of second nucleic acid and/or nucleic acid analog nanostructures may form a second layer over the first layer. The second layer is cross-linked to the first layer.

In the context of the present disclosure, the terms “nucleic acid nanostructure” refers to structures made from single-stranded deoxyribonucleic acids (ss-DNAs), DNA nanostructures, as well as structures that comprise or consist entirely of ribonucleic acid (RNA), DNA, or nucleic acid analog strands, unless specified. A “DNA nanostructure” or a “DNA origami” structure refers to a structure comprising DNA, or a DNA analog, such as PNA, and may also comprise or alternatively comprise RNA and/or RNA analogs. As such “nucleic acid nanostructures” can include nanostructures comprising ss-DNA, single-stranded RNA (ss-RNA), single stranded DNA analogs, single-stranded RNA analogs, modified single-stranded DNA or DNA analogs, and/or modified single-stranded RNA or RNA analogs, including combinations of any of the preceding.

Nucleic acids may comprise partially- or completely-modified backbones. Nucleic acid analogs include peptide nucleic acid (PNA), which includes chiral PNAs, such as gamma-PNA (γPNA, see, e.g., US Patent Application Publication No. 2017/0058325 A1, incorporated herein by reference in its entirety for its description of PNA, and particularly γPNA compositions). DNA origami has been used to prepare a large number of micromechanical structures, and choice of nucleic acids and/or nucleic acid analogs to produce a specified structure typically is well within the skill of one of ordinary skill in the art (see, e.g., Beltran S M, et al. Extending the Capabilities of Molecular Force Sensors via DNA Nanotechnology. Crit Rev Biomed Eng. 2020;48(1)1 -16; Ijäs H, et al. Dynamic DNA Origami Devices: from Strand-Displacement Reactions to External-Stimuli Responsive Systems. Int J Mol Sci. 2018;19(7):2114. Published 2018 Jul. 20; Lee C, et al. Polymorphic design of DNA origami structures through mechanical control of modular components [published correction appears in Nat Commun. 2018 Feb. 7;9(1):626]. Nat Commun. 2017; 8(1):2067. Published 2017 Dec. 12; Hunter, Philip. “Nucleic acid-based nanotechnology: The ability of DNA and RNA to fold into precise and complex shapes can be exploited for applications both in biology and electronics.” EMBO reports vol. 19,1 (2018): 13-17; and Kearney C J, et al. DNA Origami: Folded DNA-Nanodevices That Can Direct and Interpret Cell Behavior. Adv Mater. 2016; 28(27):5509-5524). DNA origami nanostructures may be modeled using available software, such as cadnano software (www.cadnano.org, see, also, Douglas, Shawn M et al. “Rapid prototyping of 3D DNA-origami shapes with caDNAno.” Nucleic acids research vol. 37, 15 (2009): 5001-6. doi:10.1093/nar/gkp436). Further, a specified structure is independent of the particular nucleic acid sequences used, but is dictated by the arrangement of complementary sequences. Although the M13 bacteriophage genome often is used as the long strand to be folded into a designated shape by the staples, any suitably long nucleotide may be utilized, provided it contains sufficient unique nucleotide sequences to permit folding into a desired shape.

Nucleic acid and/or nucleic acid analog origami structures are formed by annealing of the scaffold and staples. The individual scaffold and staples are mixed together in a suitable solvent, typically an aqueous solvent, optionally with suitable salts (e.g., MgCl₂) and buffers (e.g., Tris-acetate), and any other useful ingredients, such as chelating agents (e.g., ethylenediaminetetraacetic acid, EDTA). First, the mixture is heated, e.g. above the Tm for all ssDNA species, e.g., to 80° C., 85° C., 90° C., or 95° C., to dissociate the scaffold and staples, and then slowly cooling the mixture, e.g., to room temperature. Then the mixture is cooled, typically slowly, to anneal the complex structure. Typical cooling rates are less than 1° C. per minute, and rates may be varied within certain temperature ranges, for example in a range between 60° C. and 24° C., the rate of cooling may be slower than at higher or lower temperatures, such as less than 0.2° C. min⁻¹, less than 0.1° C. min⁻¹, less than 0.05° C. min⁻¹, less than 0.04° C. min⁻¹, less than 0.03° C. min⁻¹, less than 0.02° C. min⁻¹, or less than 0.01° C. min⁻¹. The mixture may be cooled in a typical thermal cycler apparatus that can be configured for such slow-cooling. See, e.g., U.S. Pat. No. 11,708,601 B2.Tile-based, e.g., single-stranded tile (SST) structures, in contrast to DNA origami, utilize only short sequences of ssDNA that are specifically designed to assemble into complex nanostructures. Unlike DNA origami, tile-based DNA nanostructures do not contain a long scaffold strand. However, like DNA origami, tile-based approaches such as single-stranded tiles (SSTs) and DNA “bricks” can be used to form 2D and 3D structures with either a multilayer or wireframe architecture. As such, the shell can be formed from the hybridization of nucleic acid or nucleic acid analog species as a tile structure comprising binding domains that interconnect the single-stranded nucleic acid or nucleic acid analog molecules into a defined or ordered structure, such as a structure having a periodic structure comprising two or more repeats of a specific structural motif formed from the binding of one or more single-stranded nucleic acid or nucleic acid analog molecules in a particular arrangement, optionally in combination with one or more nucleic acids or nucleic acid analogs in addition to the single-stranded nucleic acid or nucleic acid analog molecules. By SST, it is meant, without limitation, a repeated organization of nucleic acid or nucleic acid analog strands, and should not be construed as limiting to any particular structure or structure design or organization. The arrangement of the single-stranded nucleic acid or nucleic acid analog molecules may be dictated by one or more nucleobase sequences each forming a binding domain on the single-stranded nucleic acid or nucleic acid analog molecule that binds or hybridizes as a member of a binding pair with a complementary nucleobase sequence forming a binding domain on a different single-stranded nucleic acid or nucleic acid analog molecule. Complementary binding domains form a binding pair. Although they may be longer, each single-stranded nucleic acid or nucleic acid analog binding domain may be from 3 to 9, e.g., 3, 4, 5, 6, 7, 8, or 9 bases in length, typically having 100% sequence identity with its complementary binding partner. “Species” in the context of single-stranded nucleic acid or nucleic acid analog used to produce a nucleic acid and/or nucleic acid analog nanostructure refers to identical nucleic acids and nucleic acid analogs, such as single-stranded nucleic acid and/or nucleic acid analog molecules having the same sequence and structure. As such, more than one nucleic acid species may be used to produce nucleic acid or nucleic acid analog nanostructures.

The binding partners may be on a single single-stranded nucleic acid and/or nucleic acid analog strand, allowing the same strand to concatenate to form a linear, double-stranded nanostructure. More typically, and to facilitate more complex SST structures, members of binding domain pairs are located on different nucleic acid or nucleic acid analog species that form a nucleic acid and/or nucleic acid analog nanostructure. Each species of nucleic acid and/or nucleic acid analog, such as single-stranded nucleic acid or nucleic acid analog, used to produce the nucleic acid or nucleic acid analog nanostructures may include one, two, three, four, or more binding domains, dependent on the desired nucleic acid and/or nucleic acid analog nanostructures to be formed. For example, two or more different species, such as single-stranded nucleic acid and/or nucleic acid analog species, may be configured with suitable binding domains to form a nanotube of those species. Nucleic acid and/or nucleic acid analog species, that form the body of a contiguous structure, such as a linear rod or sheets, tubes, balls, or other 3D structures, typically formed from more than one rod, may be referred to as contiguous strands. Nucleic acid and/or nucleic acid analog species, that link two or more contiguous structures, such as a linear, sheet, or tube structure, may be referred to as cross-linking strands or pinning strands. Binding domains may be included in a contiguous strand, with its binding partners included in a crosslinking strand to direct specific arrangement of multiple contiguous structures, such as, for example to form an ordered bundle of tubes.

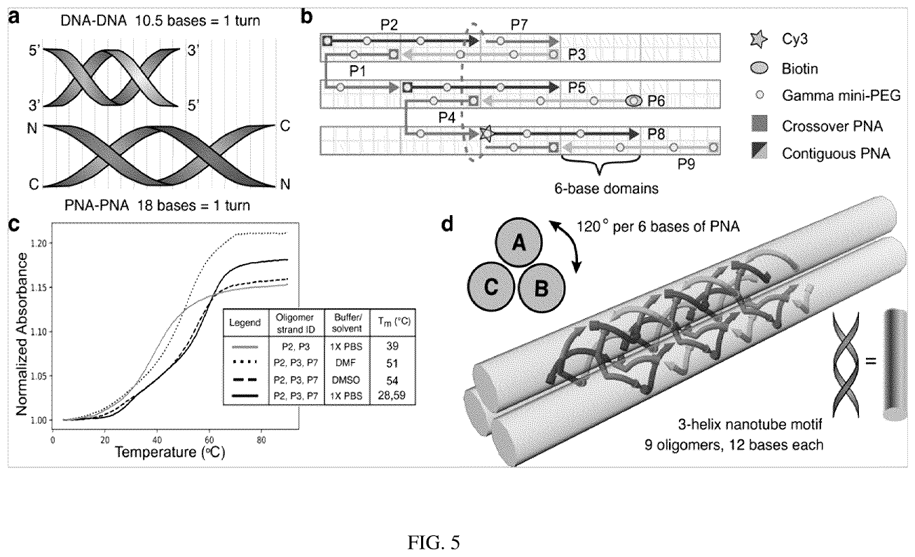

In one example, a nanostructure as described herein may comprise PNA. The following described PNA nanostructures, which may be used as a non-limiting alternative to ssDNA-based structures. FIG. 5 shows one structural difference between PNA and DNA double helices. B-form 95 DNA are right-handed double helices that rotate 34.3^{o} per base pair, or 10.5 base pairs per helical turn. To account for this property and prevent undesired pre-stress, DNA SST designs usually aim for 10.4-10.7 bases per helical revolution (see International Patent Application Publication No. WO 2020/205588, which is incorporated herein by reference in its entirety). Unlike DNA, PNA double helices are reported to have 18 base pairs per turn. Therefore, a motif with 18 bases-long repeat unit to make a beam consisting of three interwoven double helices (e.g., rods) can be prepared. The design shown in FIG. 5 (b) is based on repeating tubular units where the number of unique oligomers required to form the individual units are 3X the number of helices in the corresponding bundles (e.g., 9 oligomers for a 3-rod (e.g., helix) beam). The constituent strands fall into one of two categories: two-thirds of them (6 oligomers) are contiguous strands that are arranged linearly and the other one-third (3 oligomers) are crossover strands that each form a crossover from one rod (e.g., helix) to a neighboring rod (e.g., helix). Each yPNA sequence may contain 3 gamma modifications with (R)-diethylene glycol (mini-PEG) at the 1, 4 and 8 positions (FIG. 5 (b), gray dots). In order to immobilize and visualize PNA nanostructures using fluorescence microscopy, N-terminal functionalization of select strands with biotin (FIG. 5 (b), oval) and Cy3 (FIG. 5 (b), star) can be performed. Each oligomer consists of 12 bases, which follows a 6+6 domain-binding pattern. For PNA double helices with 18 base pairs per full helical turn, 6 base pairs correspond to 120° rise in helical rotation in a triangular-sectioned beam (FIG. 5 (b and d)).

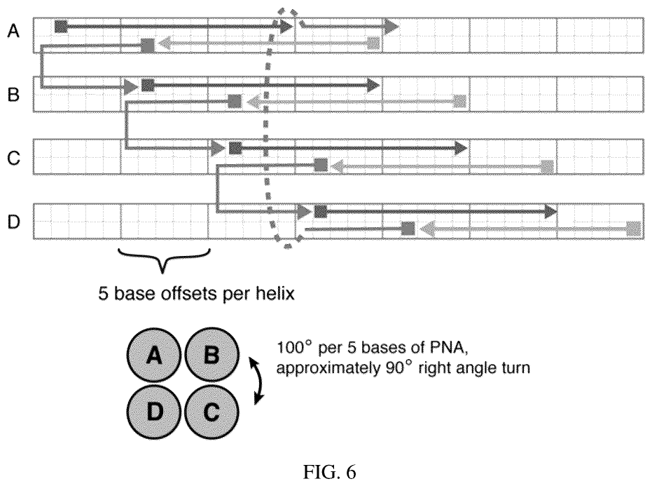

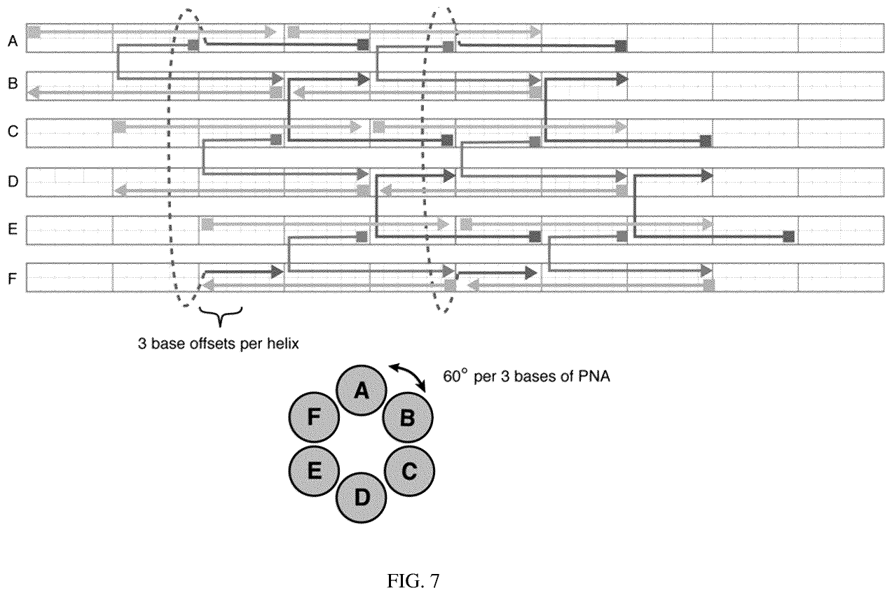

A four-rod (e.g., helix) beam design may be provided. An example of such a design is depicted in FIG. 6. The four-rod (e.g., helix) beam design has a repeating unit that is 20 bases long, which assumes 20 bases per turn (close to PNA's measured 18 bases per turn). A rod-to-rod (e.g., helix-to helix) offset of five bases allows for approximately a 90 degree rotation. This design consists of 12 distinct PNA strands that are 13 and 14 bases in length. However if made radially symmetric, only four distinct strands are needed, with three used in each repeat. A six-rod (e.g., helix) beam can also be provided. An example of such a design is depicted in FIG. 7. For example, the six-rod (e.g., helix) beam design may have a repeating unit that is 18 bases long, which assumes 18 bases per turn (equal to PNAs measured 18 bases per turn). Rod-to-rod (e.g., helix-to-helix) offset of three bases allows for a 60 degree rotation between rods (e.g., helices). This design consists of 12 distinct PNA strands that are 18 bases long.

The nucleic acid and/or nucleic acid analog nanostructures may be formed from a combination of single-stranded nucleic acid or nucleic acid analog species and one or more nucleic acid species and/or nucleic acid analog species, which confer different properties to the nucleic acid or nucleic acid analog nanostructure.

The nucleic acid and/or nucleic acid analog nanostructures may be prepared in any suitable solvent. A surfactant, such as an anionic surfactant, may be added to the solvent, which may improve structural uniformity and stability. The nucleic acid and/or nucleic acid analog nanostructures are prepared by mixing the precursors, that is the nucleic acid and/or nucleic acid analog species in appropriate stoichiometric ratios with the solvent, and complexing the precursors to form the SST structure. Addition of the precursors to the solvent may be enough to form a desired the SST structure. It may be that each precursor is already stored in a suitable solvent prior to mixing and complexing, such as a solvent used to produce the precursor. The precursors may be added in an organized, step-wise manner to first form intermediate structures, and later to form higher-complexity structures. The precursors may be mixed together and maintained at any temperature or series of temperatures useful to make a nucleic acid and/or nucleic acid analog nanostructure therefrom. The precursors may be maintained at a single temperature to form the nucleic acid and/or nucleic acid analog nanostructures. The solvent containing the precursors may be heated to a temperature above which the precursors do not bind, such as a temperature above the Tm of one or more, or all, binding domain pairs in the mixture, followed by cooling the mixture to a temperature at which the precursors bind, such as a temperature below the Tm of one or more, or all, binding domain pairs in the mixture. The temperature may be lowered in a step-wise fashion to allow for partial binding of certain precursors before binding of other precursors. Certain precursors may be added at one time point, and others at a second time point, optionally combined with manipulation of the temperature as described to order the assembly of the nucleic acid or nucleic acid analog nanostructures. Additional reagents, such as solvents, surfactants, emulsifiers, lipids, water and salts, etc. may be included in the mixture, or added to the mixture to further manipulate and facilitate a desired nucleic acid and/or nucleic acid analog nanostructure assembly scheme.

Solvents useful in assembly of the nucleic acid and/or nucleic acid analog nanostructures described herein are solvents that permit assembly of the structures by hybridization of the recognition domains of the single-stranded nucleic acid and/or nucleic acid analog strands. As such, useful solvents may include a solvent that does not interfere with hydrogen-bonding donor/acceptor activity to the extent that it interferes with assembly and/or stability of the SST structure. Such solvents may be a polar aprotic solvent or a polar aprotic organic solvent. Polar aprotic solvents are solvents that lack an acidic hydrogen and therefore are not hydrogen bond donors, examples of which include, without limitation: dimethylsulfoxide (DMSO), dimethylformamide (DMF), hexamethylphosphoramide (HMPA, which not organic), dichloromethane, N-methylpyrrolidone, tetrahydrofuran, acetonitrile, propylene carbonate, pyridine, and ethyl acetate, including combinations of two or more of any of the preceding. Other useful solvents may include: dimethylacetamide, valerolactone, 2,5-dimethyltetrahydrofuran.Ligands may be attached to any shell described herein to facilitate attachment of the shell to a target cell, tissue, or other structure, or to assist in assembly of the shell structure. A ligand can be attached to the described nucleic acid and/or nucleic acid analog nanostructure by any suitable method, either directly by covalent linkage or indirectly using suitable ligands, such as the biotin/streptavidin conjugation, and may be attached, directly or indirectly to a pendant portion or tether of a staple used to prepare the nucleic acid and/or nucleic acid analog nanostructure. The sequence of a nucleic acid-based ligand, such as an aptamer, may be incorporated directly within the pendant portion or tether of a staple used to prepare the nucleic acid and/or nucleic acid analog nanostructure.

The term “ligand” refers to a binding compound for a specific target, referred to as its binding partner. The molecule can be a cognate receptor, a protein a small molecule, a hapten, or any other relevant molecule. The term “antibody” refers to an immunoglobulin, derivatives thereof which maintain specific binding ability, and proteins having a binding domain which is homologous or largely homologous to an immunoglobulin binding domain. As such, the antibody operates as a ligand for its cognate antigen, which can be virtually any molecule. Natural antibodies typically comprise two heavy chains and two light chains and are bi-valent. The interaction between the variable regions of heavy and light chain forms a binding site capable of specifically binding an antigen (e.g., a paratope). The term “V_{H}” refers to a heavy chain variable region of an antibody. The term “V_{L}” refers to a light chain variable region of an antibody. Antibodies may be derived from natural sources, or partly or wholly synthetically produced. Many antibodies and fragments thereof are available from commercial sources. An antibody may be monoclonal or polyclonal. The antibody may be a member of any immunoglobulin class, including, for example and without limitation, any of the human classes: IgG, IgM, IgA, IgD, and IgE and camelid.

“Lectins” are a group of proteins from non-immune origins that bind carbohydrates and agglutinate animal cells. They exhibit extremely high binding affinities for specific sugars, and can be used to target specific cell types expressing their binding partner, including carbohydrates, polysaccharides, glycoproteins, and glycolipids. They serve a variety of functions in their natural setting, but can be a powerful tool when used to target their binding partner. Lectins can agglutinate cells and/or precipitate complex carbohydrates and, as such, have served as a powerful tool for biomedical research and clinical utility, including, carbohydrate studies, fractionation of cells and other particles, lymphocyte subpopulation studies, mitogenic stimulation, blood group typing, and histochemical studies. They are isolated from a wide variety of natural sources, both plant and animal. Concanavalin A (Con A) is a broadly-studied lectin that binds α-D-mannosyl and α-D-glucosyl residues. Peanut agglutinin targets Galβ1-3GaINAcα1 -Ser/Thr and, e.g., inhibits T-cell activity and can be used to distinguish lymphocyte subsets. Many other lectins are broadly-known and characterized, and can be obtained from commercial sources.

Ligands, also referred to as binding reagents, having limited cross-reactivity are generally preferred. In certain embodiments, suitable ligands include, for example, polypeptides, such as for example, lectins or antibodies, monoclonal antibodies, or fragments, derivatives, or analogs thereof, including without limitation: Fv fragments, single chain Fv (scFv) fragments, Fab₁ fragments, F(ab′)₂ fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((ScFv)₂ fragments), diabodies, triabodies, tetrabodies, which typically are covalently linked or otherwise stabilized (e.g., leucine zipper or helix stabilized) scFv fragments, di-scFv (dimeric single-chain variable fragment), single-domain antibody (sdAb), or antibody binding domain fragments and other binding reagents including, for example, bi-specific T-cell engagers (BiTEs), aptamers, template imprinted materials, and organic or inorganic binding elements. In exemplary embodiments, a ligand specifically interacts with a single motif or epitope. In other embodiments, a ligand may interact with several structurally-related motifs or epitopes.

The term “antibody fragment” refers to any derivative of an antibody which is less than full-length. In exemplary embodiments, the antibody fragment retains at least a significant portion of the full-length antibody's specific binding ability. Examples of binding reagents, including antibody fragments, but are not limited to, Fab, Fab′, F(ab′)₂, Fv, Fd, dsFv, scFv, diabody, triabody, tetrabody, di-scFv (dimeric single-chain variable fragment), bi-specific T-cell engager (BiTE), single-domain antibody (sdAb), or antibody binding domain fragments. The antibody fragment may be produced by any means. For instance, the antibody fragment may be enzymatically or chemically produced by fragmentation of an intact antibody, or it may be recombinantly or synthetically produced. The antibody fragment may optionally be a single chain antibody fragment. Alternatively, the fragment may comprise multiple chains which are linked together, for instance, by disulfide linkages. The fragment may also optionally be a multi-molecular complex. A functional antibody fragment may consist of at least about 50 amino acids or at least about 200 amino acids. Antibody fragments also include miniaturized antibodies or other engineered binding reagents that exploit the modular nature of antibody structure, comprising, often as a single chain, one or more antigen-binding or epitope-binding (e.g., paratope) sequences and, at a minimum, any other amino acid sequences needed to ensure appropriate specificity, delivery, and stability of the composition (see, e.g., Nelson, A L, “Antibody Fragments Hope and Hype” (2010) MAbs 2(1):77-83).

Also provided herein is a method of preparing a ruggedized cell. A plurality of first nucleic acid and/or nucleic acid analog nanostructures are anchored to a surface of a live cell. A shell is formed by crosslinking the plurality of first nucleic acid and/or nucleic acid analog nanostructures with a plurality of second nucleic acid and/or nucleic acid analog nanostructures using a plurality of nucleic acids and/or nucleic acid analog staples, wherein the plurality of first nucleic acid and/or nucleic acid analog nanostructures are attached to the plurality of second nucleic acid and/or nucleic acid analog nanostructures. The live cell can be any of the cells described herein. The plurality of first nucleic acid and/or nucleic acid analog nanostructures can be any of the first nucleic acid and/or nucleic acid analog nanostructures described herein. The plurality of second nucleic acid and/or nucleic acid analog nanostructures can be any of the first nucleic acid and/or nucleic acid analog nanostructures described herein. The plurality of nucleic acids and/or nucleic acid analog staples can be any of the nucleic acids and/or nucleic acid analog staples described herein.

The method of forming the shell may comprise forming a plurality of first nucleic acid and/or nucleic acid analog nanostructure beams comprising the plurality of nucleic acids and/or nucleic acid analog staples. The beams may comprise one or more single-stranded scaffold nucleic acids or nucleic acid analogs. The plurality of first nucleic acid and/or nucleic acid analog nanostructure beams comprising the plurality of nucleic acids and/or nucleic acid analog staples may be then be anchored to the cell. The plurality of second nucleic acid and/or nucleic acid analog nanostructures comprising a plurality of nucleic acids and/or nucleic acid analog staples may be formed. The nucleic acids and/or nucleic acid analog staples of the plurality of second nucleic acid and/or nucleic acid analog nanostructures are complementary to the plurality of nucleic acids and/or nucleic acid analog staples of the plurality of first nucleic acid and/or nucleic acid analog nanostructure beams. The plurality of first nucleic acid and/or nucleic acid analog nanostructure beams and the plurality of second nucleic acid and/or nucleic acid analog nanostructures are cross-linked through the hybridization of the staples to link two or more of the first nucleic acid and/or nucleic acid analog nanostructure beams to the second nucleic acid and/or nucleic acid analog nanostructures to form the cell.

Forming the shell may also comprise forming a first layer about the cell with the plurality of first nucleic acid and/or nucleic acid analog nanostructures, and forming a second layer over the first layer with the plurality of second nucleic acid and/or nucleic acid analog nanostructures. The first layer is cross-linked to the second layer.

Also provided herein is a method of preparing a graft for tissue replacement or repair in a patient. In this method, the ruggedized cells can be deposited onto the surface of a polymer matrix structure. The ruggedized cells can be suspended in a liquid, e.g., cell growth media and deposited through an electrospray deposition technique or a printing technique, such as ink-jet printing. Following deposition, the cells can be cultured on the polymer matrix structure under conditions for differentiating the cells into cells of the tissue to be repaired or replaced and cultured to expand the number of cells on the polymer matrix structure.

A number of biocompatible, biodegradable elastomeric polymers and (co)polymers are known and have been established as useful in preparing cell growth support matrices. Non-limiting examples of a bioerodible polymer useful for the polymeric matrix scaffold, include but are not limited to: a polyurethane, a polyester, a polyester-containing copolymer, a polyanhydride, a polyanhydride-containing copolymer, a polyorthoester, and a polyorthoester-containing copolymer. In one aspect, the polyester or polyester-containing copolymer is a poly(lactic-co-glycolic) acid (PLGA) copolymer. In other aspects, the bioerodible polymer is selected from the group consisting of poly(lactic acid) (PLA); poly(trimethylene carbonate) (PTMC); poly(caprolactone) (PCL); poly(glycolic acid) (PGA); poly(glycolide-co-trimethylenecarbonate) (PGTMC); poly(L-lactide-co-glycolide) (PLGA); polyethylene-glycol (PEG-) containing block copolymers; and polyphosphazenes. Additional bioerodible, biocompatible polymers include: a poly(ester urethane) urea (PEUU); poly(ether ester urethane)urea (PEEUU); poly(ester carbonate)urethane urea (PECUU); poly(carbonate)urethane urea (PCUU); a polyurethane; a polyester; a polymer comprising monomers derived from alpha-hydroxy acids such as: polylactide, poly(lactide-co-glycolide), poly(l-lactide-co-caprolactone), polyglycolic acid, poly(dl-lactide-co-glycolide), and/or poly(l-lactide-co-dl-lactide); a polymer comprising monomers derived from esters including polyhydroxybutyrate, polyhydroxyvalerate, polydioxanone, and/or polyglactin; a polymer comprising monomers derived from lactones including polycaprolactone; or a polymer comprising monomers derived from carbonates including polycarbonate, polyglyconate, poly(glycolide-co-trimethylene carbonate), or poly(glycolide-co-trimethylene carbonate-co-dioxanone).

The following Examples illustrate various embodiments of the invention. However, it is to be understood that the invention is not limited to these specific embodiments.

### EXAMPLES

The bioengineering application of cells, such as cell printing and multicellular assembly, are limited by cell damage and death due to harsh environments and mechanical conditions. A nanoshell encapsulation strategy is provided that targets the cell-surface glycocalyx, which utilizes two layers of DNA nanorods by sequentially recruiting and crosslinking them onto cell membranes under physiological conditions. It was shown that the DNA origami nanoshell modulates the biophysical properties of cell membranes by enhancing membrane stiffness and lowering lipid fluidity. The nanoshell also served as armor to protect cells and improve their viability against mechanical stress from osmotic imbalance, centrifugal forces, and fluid shear stress. In addition, the nanoshell allowed for mediated cell-cell interactions for effective and robust multicellular assembly. Thus, the nanoshell is a cellular protection strategy and also provides a platform for cell and cell membrane manipulation.

### Materials and Methods

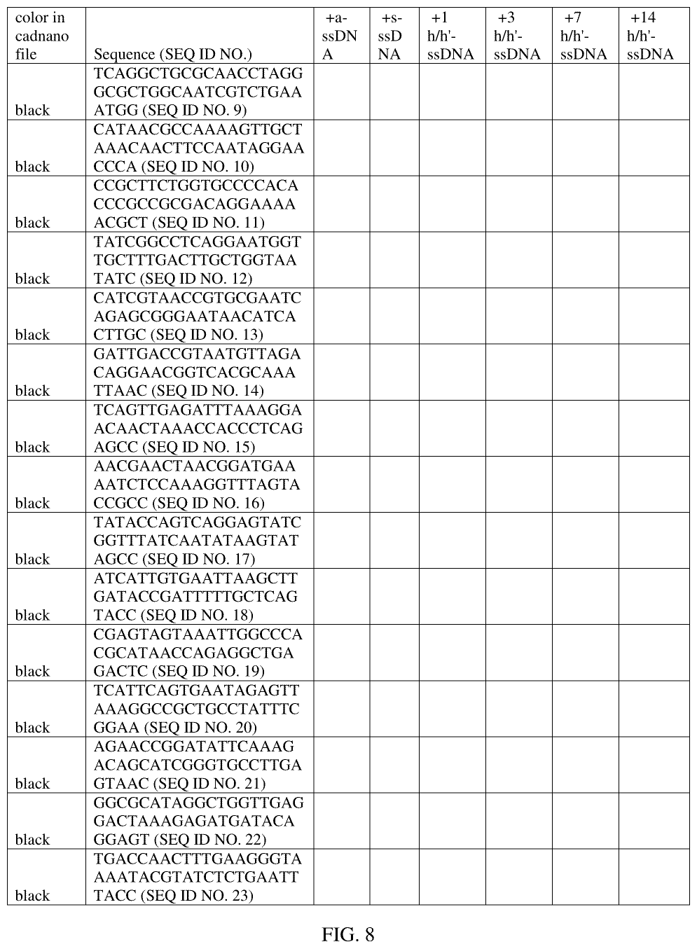

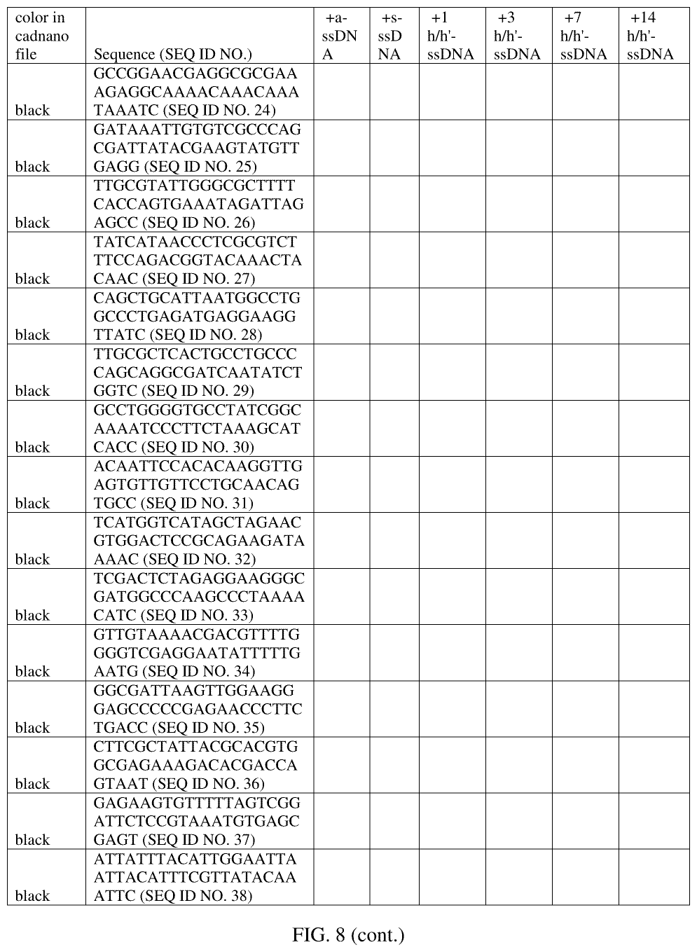

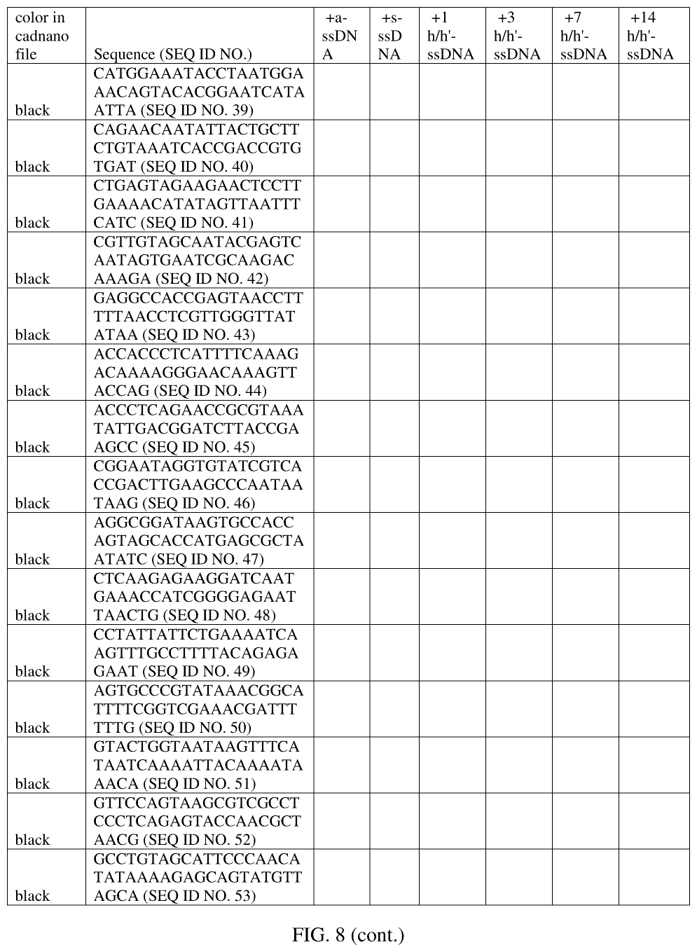

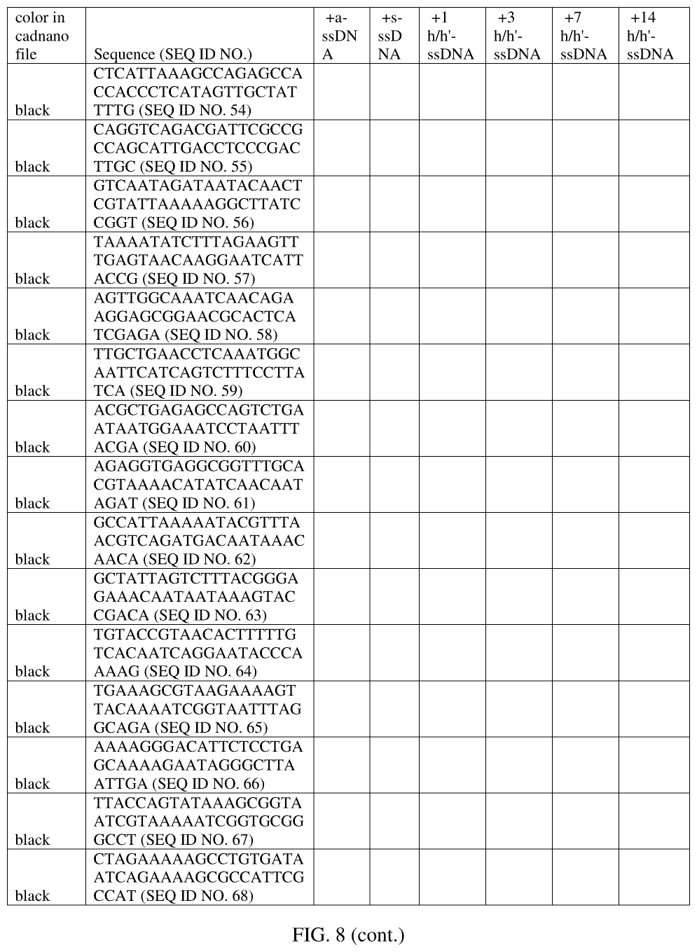

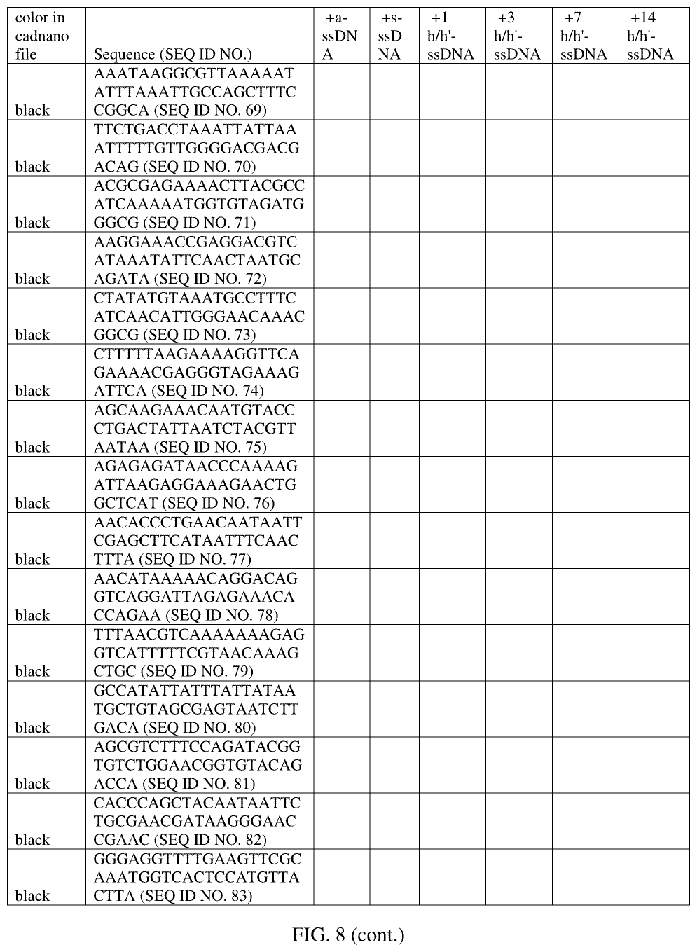

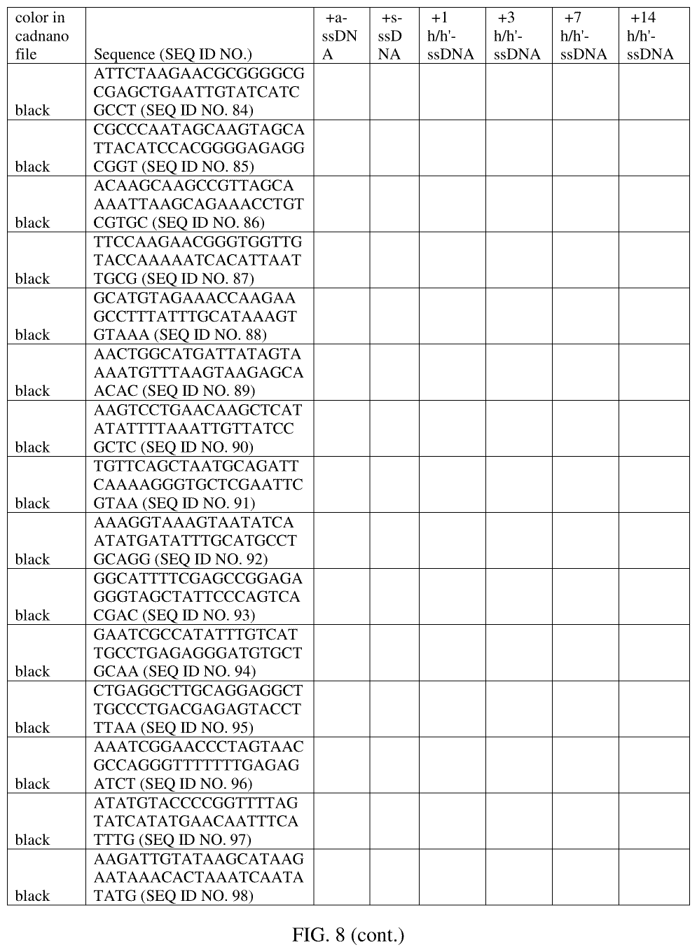

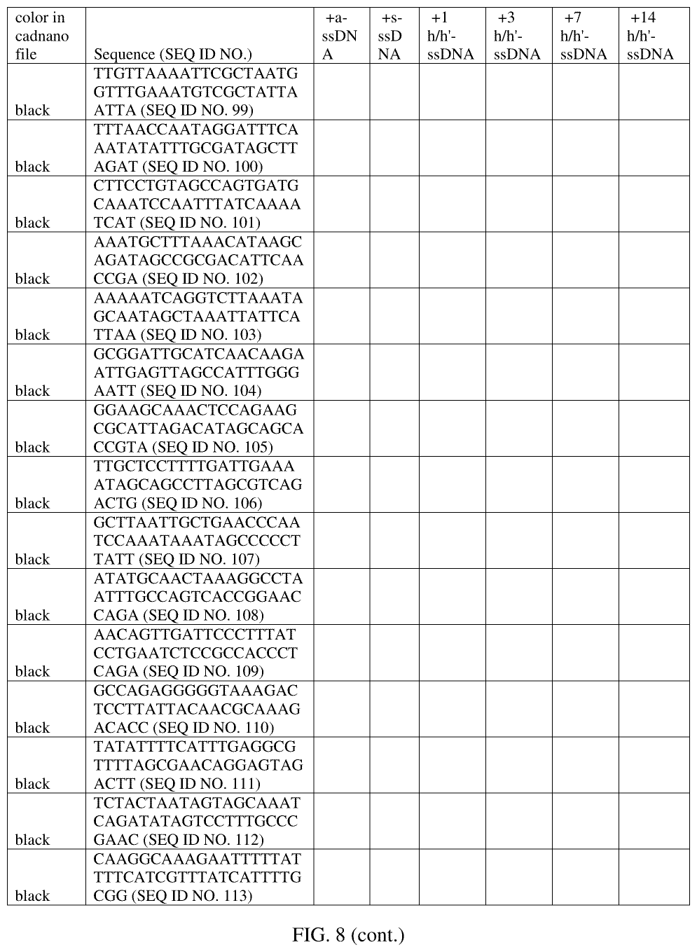

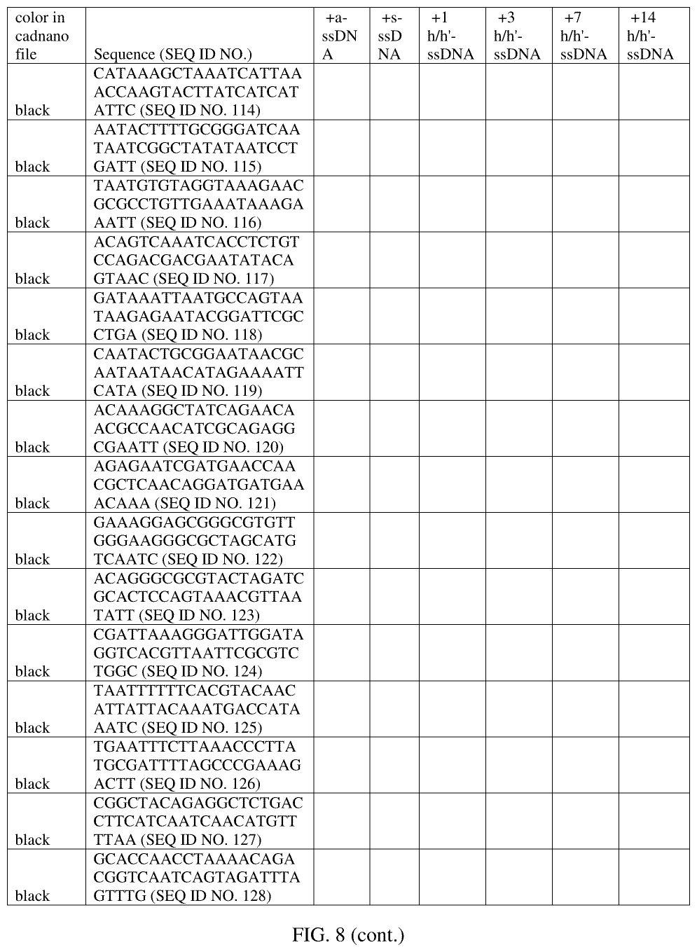

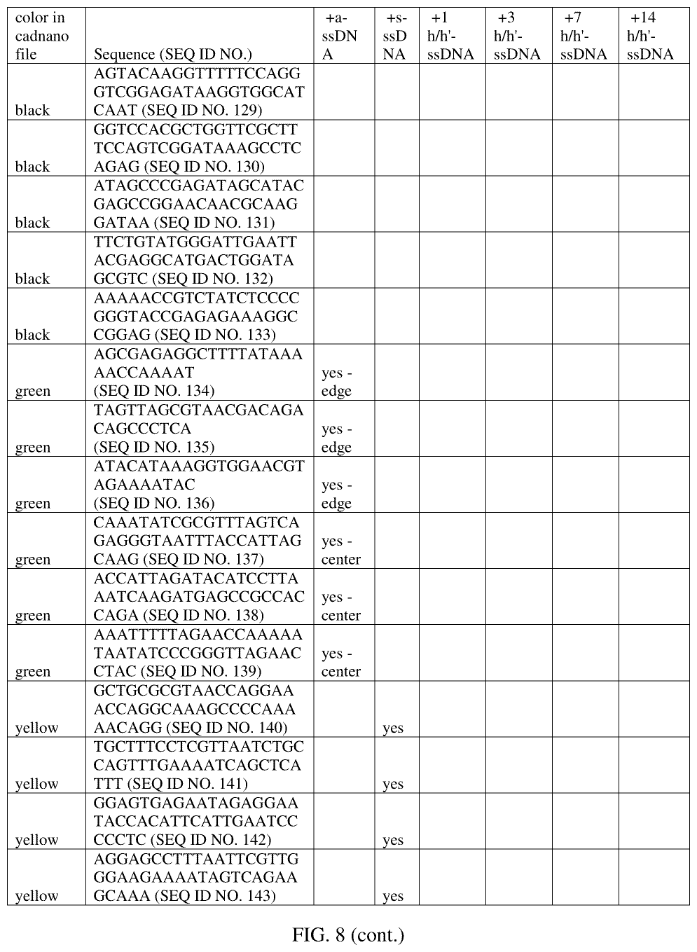

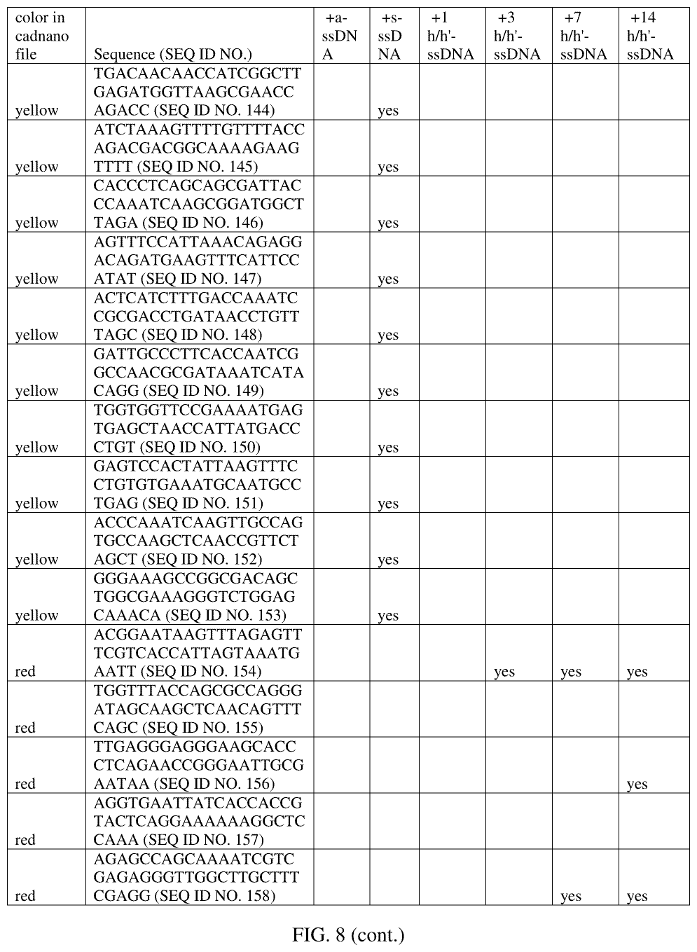

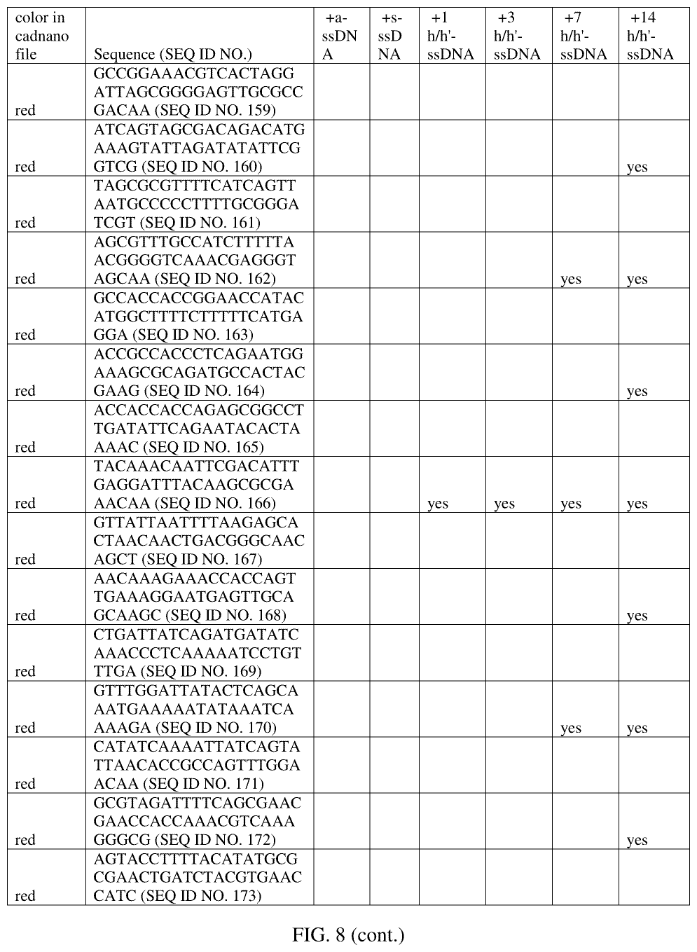

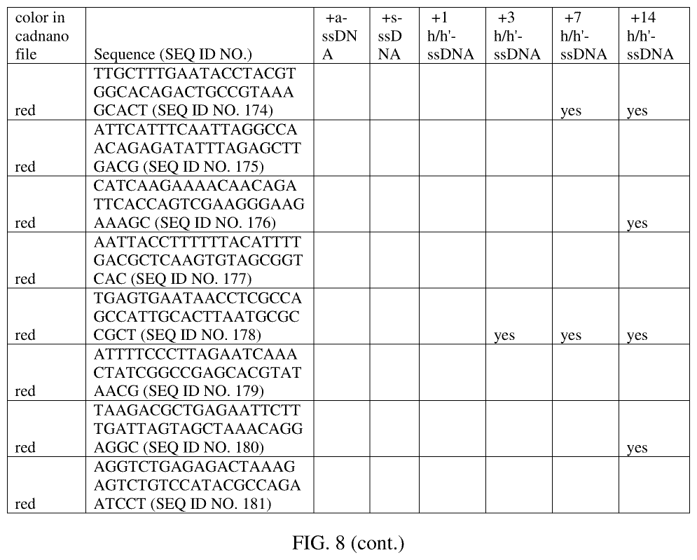

Materials, Reagents, and Equipment: DNA oligos (Table 1) were purchased from Integrated DNA Technologies (Coralville, IA). Additional DNA oligos for DNA nanorods can be found in FIG. 8. DBCO-cy5 and DBCO-Sulfo-NHS-Ester, Sodium chloride and paraformaldehyde and PEG 8000 were purchased from Sigma-Aldrich. Streptavidin-Alexa Fluor 488 and 647 conjugates were purchased from Invitrogen. Roswell park memorial institute (RPMI) 1640 medium, Dulbecco's phosphate-buffered saline (DPBS) was purchased from Corning. Bovine serum albumin (BSA) was purchased from Fisher BioReagents. AFM tips were purchased form NanoAndMore. Micropipettes (5 micron (μm) in diameter) were purchased from World Precision Instruments. Membrane lipid dye DiD and DNase I were purchased from ThermoFisher. ReadyProbes Cell viability imaging kit (Blue/Green) and SYBR Safe DNA gel stain were purchased from Invitrogen. Endothelial cell growth medium-2 BulletKit was purchased from Lonza.

TABLE 1
List of Functional DNA oligos

| Name of oligos | Sequence (SEQ ID NO.) |
|---|---|
| Anchoring-ssDNA (a-ssDNA) modifier | 5′/TT CAGTCAGTCAGTCAGTCAGT/3′ |
| | (SEQ ID NO. 1) |
| staining-ssDNA (s-ssDNA) modifier | 5′/TT GAGAGCAGACCTGGAACTCG/3 |
| | (SEQ ID NO. 2) |
| hybridization-ssDNA modifier (h- | 5′/TT GTGACATACCTTCGGAGCAT/3′ |
| ssDNA, for rod A) | (SEQ ID NO. 3) |
| hybridization-ssDNA complementary | 5′/TT ATGCTCCGAAGGTATGTCAC/3′ |
| modifier (h′-ssDNA, for rod B) | (SEQ ID NO. 4) |
| 5′NH₂-a′-ssDNA | /5AmMC6/TT ACTGACTGACTGACTGACTG |
| | (SEQ ID NO. 5) |
| Biotin-s′-ssDNA | /5BiosG/TT CGAGTTCCAGGTCTGCTCTC |
| | (SEQ ID NO. 6) |
| Cy5-s′-ssDNA modifier | /5Cy5/TT CGAGTTCCAGGTCTGCTCTC |
| | (SEQ ID NO. 6) |
| Cy3-s′-ssDNA modifier | /5Cy3/TT CGAGTTCCAGGTCTGCTCTC |
| | (SEQ ID NO. 6) |
| Chol-a′-ssDNA | 5′/Chol-TEG/ACTGACTGACTGACTGACTG/3′ |
| | (SEQ ID NO. 7) |
| Chol-a-ssDNA | 5′/Chol-TEG/CAGTCAGTCAGTCAGTCAGT/3′ |
| | (SEQ ID NO. 8) |

Synthesis and purification of biotinylated DNA nanorods: DNA nanorods were folded from M13mp18 scaffold (Bayou Biolabs) together with 173 staple strands (Rod A) and 170 staples (Rod B). For annealing, 20 nanomolar (nM) of the scaffold and 100 nM of the staples were mixed in TAE buffer with 12.5 millimolar (mM) magnesium chloride (MgCl₂). The biotin-labeled strands were added pre-annealing with the same concentration as other staples. The mixture was heated to 80 degrees Celsius (° C.) for 5 minutes and gradually cooled down to 4° C. within 45 hours specifically, 80° C.-65° C., 0.1° C. per 24 seconds; 65° C.-24° C., 0.1° C. per 378 seconds; 24° C.-4° C., 0.1° C. per 18 seconds. After annealing, the solutions were collected and precipitated by centrifugation at 10500 g for 25 minutes in a buffer containing 7.5% PEG 8000, 10 mM MgCl₂, 255 mM NaCl, 22.5 mM Tris, 10 mM acetic acid, and 1 mM EDTA. The supernatant was removed and the pellet was resuspended to 5 nM using DPBS buffer with 12.5 mM MgCl₂. DNA nanorods were then administrated to cells within 30 min after purification.

Agarose gel electrophoresis and stability test: 10 microliters (μl) of 10 nM purified DNA nanorod samples were analyzed by electrophoresis in 2% agarose gel in TBE buffer with 12.5 mM MgCl₂ at 90 Volts (V) and room temperature (RT) for 1 hour. To study the structural stability of DNA nanorods in cell media, DNA Rod A were incubated in EGM-2, RPMI+10% FBS at 37° C. for 6 hours and 30 hours before running the gel. To test the stability of Rod A and Rod B aggregate stability, Rod A and Rod B were resuspended in RPMI+10% FBS at 20 nM and were mixed and incubated at 37° C. for 30 minutes and 30 hours before running the gel. The gels were stained with 1×SYBR Safe DNA gel stain and imaged with Biorad ChemiDoc Imaging System

Atomic force microscopy (AFM): 10 μl of 0.5 nM of purified DNA nanorod samples in DPBS with 12.5 mM MgCl₂ was deposited onto a freshly cleaved mica surface, incubated at RT in a humid chamber for 5 minutes. The sample was washed three times with 20 μl DI water and thoroughly blow-dried with nitrogen after each washing. AFM scans were performed using MFP-3D-BIO AFM (Asylum Research) with a 5 nM AFM tip in tapping mode.

Synthesis of 5′DBCO-a′-ssDNA: The 5′NH2-a′-ssDNA oligos were incubated overnight in DPBS with DBCO-sulfo-N-hydroxysuccinimidyl ester (DBCO-Sulfo-NHS-Ester) at a 1:10 molar ratio under agitation at RT for 30 minutes. After incubation, the reaction mixture was dialyzed five times against DPBS using Amicon Ultra Centrifugal filters (molecular weight cut-off, 3 kiloDaltons (kDa)) to remove unconjugated DBCO-Sulfo-NHS-Ester. The above-described conjugation and purification procedures were repeated twice in total to increase the conjugation efficiency. The final concentration of 5′DBCO-a′-ssDNA was adjusted to 500 μM with DPBS.

Cell culture: Jurkat, clone E6-1, were purchased from ATCC. Cells were cultured in RPMI+10% FBS at 37° C. with 5% CO₂ and were maintained at a cell density between 100,000 cells per milliliter (cells/ml) and 3,000,000 cells/ml. Human umbilical vein endothelial cells (HUVECs) were purchased from the Lonza. Cells were cultured in EGM-2 at 37° C. with 5% CO₂. HUVECs of passage 3-5 were plated into 96-well culture plates at a density of 50,000 cells per well, one day prior to the experiment. Both cells have been tested negative for mycoplasma contamination.

Synthesis of DNA nanoshell on membrane through click chemistry and ssDNA hybridization: In the first step, a′-ssDNA was immobilized on glycocalyx. Azide ligands were firstly covalently incorporated onto glycocalyx 24 hours before experiment, through metabolic glycan labeling using an azido monosaccharide, N-azidoacetylmannosamine-tetraacylated (Ac4-ManNAz). Specifically, Ac4ManNAz was diluted in culture medium to a final concentration of 50 μM and administered to cells 24 hours before experiment. DBCO-a′-ssDNA was administrated to Jurkat cells at a final concentration of 50 μM and 100 μl in volume and incubated for 1 hour. Cells were then washed with DPBS+0.1° /0 BSA as washing buffer to remove excess DBCO-a′-ssDNA using centrifugation at 200 g for 3 minutes. Next, 100 μl of 5 nM DNA Rod A in DPBS with 12.5 mM MgCl₂ were administrated to cells and incubated for 30 minutes. Then 100 μl of 5 nM DNA Rod B in DPBS with 12.5 mM MgCl₂ were administrated to cells and incubated for 30 minutes. Cells then were washed and resuspended in DPBS or RPMI+10% FBS buffers for later experiments.

Examination of the cell surface retention time of the nanoshell with flow cytometry: In this experiment, only DNA Rod A were biotinylated and fluorescently labeled. Cells decorated with only Rod A and with both Rod A and Rod B were incubated at three conditions: 4° C. for 30 minutes, (ii) 37° C. for 30 minutes and (iii) 37° C. for 3 hours, with RPMI1640+10% FBS as buffer. After incubation, cells were collected and fixed using 1 milliliter (ml) of 4% of paraformaldehyde with 10 minutes at RT. DPBS was used for washing. DNA Rod A were then stained with streptavidin-AF647 and cells were washed. Finally, cells were analyzed using flow cytometry and fluorescence intensity of AF647 was examined by FACS.

Degradation of DNA nanorods and nanoshells on cell membranes: DNA Rod A-coated cells and nanoshell-coated cells (1,000,000 cells/m1) were incubated at 37° C. for 30 minutes with a concentration of DNase I at 20 units (U) and with DPBS as buffer. After incubation, cells were washed and imaged.

Examination of nanoshell internalization: Two groups of cells, Rod A-coated cells and nanoshell-coated cells were prepared and incubated at 37° C. for 30 minutes. In both groups, only Rod A were fluorescently labeled with AF647. After incubation, cells were fixed using 4% paraformaldehyde. DNase I at 20 U was introduced to remove cell-surface DNA rods. The background fluorescence noises 13 were removed and the internalization signal intensity were quantified using a previously reported computational pipeline (Wijesekara et al. “Accessing and Assessing the Cell-Surface Glycocalyx Using DNA Origami”, Nano Lett, 2021, 21(11): 4765-4773).

Quantification of fluorescence signal distribution on cell border: The fluorescence signals on cell borders were acquired using National Institutes of Health Image J by manually circling cell borders and recording the intensity (Schneider et al. “NIH Image to ImageJ: 25 Years of Image Analysis”, Nat. Methods, 2012, 9(7): 671-675). The data were then analyzed by a custom Python 3.8 code using SciPy where the signal valley was defined as a fluorescence signal that was lower than 0.4, and the number and length of signal valleys were quantified (Virtanen et al., “SciPy 1.0: Fundamental Algorithms for Scientific Computing in Python”, Nat. Methods, 2020, 17(3): 261-272).

Examination of membrane lipid fluidity with FRAP: Live native cells, Rod A-coated cells and nanoshell-coated cells were observed under confocal laser scanning microscope (LSM 880 with Airyscan, ZEISS). Cell membranes were stained with DiD lipid dye (red) and DNA Rod A were stained with AF488 (green). Region of interest (ROI) was selected and set to be photobleached. The pinhole size for photobleaching was 2 μm. After photobleaching, the relation of fluorescence recovery with time was recorded and analyzed to calculate the mobile fraction, t_{0.5} and recovery constant by ZEN (ZEISS).

Examination of cell membrane elastic modulus with micropipette aspiration: Live native cells, Rod A-coated cells and nanoshell-coated cells were deformed by glass micropipettes. Specifically, the micropipette was connected to tubing that ran to a water-filled reservoir with controllable height. The tip of the micropipette was then placed near a cell and a suction pressure was applied within the micropipette. The pressure difference deformed the cell and a membrane protrusion was formed. The length of the protrusion, the aspirated length, was recorded. Pressure difference and aspirated length were fitted linearly by forcing the fitted line to pass through the origin. Elastic modulus was calculated using the model and equation by Theret et al., assuming the cell as a continuum-medium model with homogeneity (Theret et al., “The Application of a Homogeneous Half-Space Model in the Analysis of Endothelial Cell Micropipette Measurements”, J. Biomech. Eng., 1988, /10(3):190-199).

Protective effect of the nanoshell against osmotic swelling and centrifugal forces: In the osmotic swelling assay, live native cells, Rod A-coated cells and nanoshell-coated cells were diluted to a concentration of 1,000,000 cells/ml. Cell membranes were stained by DID lipid dye (red). Cells were then centrifuged and resuspended in 1 ml of NaCl solutions (0%, 0.3%, 0.6%, 0.9%). After 10 minutes of incubation at 37° C., cells were collected and imaged by a fluorescence microscope (EVOS). The sizes of cells were analyzed using Image J software by applying the same threshold to red areas in all images. To test cell viability, when incubating cells, NaCl buffers were mixed with cell viability imaging kit solutions. After incubation, cells were imaged on EVOS at 40x in both Cy5 and GFP channels. In the centrifugal assay, cells (1,000,000/ml) were centrifuged (centrifuge 5804R, Eppendorf) five times at 4° C. with a speed of 100 g, 1500 g and 3000 g, respectively. After each centrifugation, cells were resuspended by 1 ml of DPBS. Cells were then incubated in cell viability imaging kit solutions at 37° C. for 10 minutes, followed by fluorescence microscopy imaging. Dead cells were stained green. All cells were stained blue. Cell viability was calculated as the ratio of live cell/all cells.

Protective effect of the nanoshell against ex vivo syringe-needle ejection-induced fluid shear stress: Live native cells, Rod A-coated cells and nanoshell-coated cells were collected at 500,000 cells/ml in RPMI+10% FBS cell medium. 5 ml of cell suspension was slowly loaded into a 20 ml syringe (BD Biosciences). A 30 gauge needle (Jensen Global) was then attached to the syringe. An automated syringe pump (New Era NE-1000) was prepared and the pumping rate was adjusted to 0.6, 5.8 and 8.4 milliliters per minute (ml/min), respectively, corresponding to 10, 97 and 140 microliters per second (μl/s) flow rate. At room temperature, under each pumping rate, cells were ejected through the needle and suspension was collected. The process was repeated 10 times. Cell viability was examined using ReadyProbes Cell viability imaging kit.

DNA nanoshell-mediated cell assembly: First, two groups of Jurkat cells were fluorescently stained with CellTracker Green CMFDA Dye and CellTracker Deep Red Dye, respectively. One group of cells were incubated with cholesterol-a-ssDNA and the other group of cells were incubated with cholesterol-a′-ssDNA. Both incubations were 1 hour long. For ssDNA-mediated cell assembly, two group of cells (cell surface immobilized with cholesterol-a-ssDNA and cholesterol-a′-ssDNA) were mixed at a ratio of 1:100 (10,000 cells/ml: 1,000,000 cells/ml) at 37° C. for 30 minutes. For DNA nanoshell-mediated cell assembly, two groups of cells were firstly incubated with Rod A (3 a-ssDNA, 14 h-ssDNA) and Rod B (3 a-ssDNA, 14 h′-ssDNA, where the positions of a-ssDNA are the same of rod A) at 37° C. for 30 minutes, respectively, followed by centrifugation washing. Then two groups of cells were mixed at 1:100 ratio for another 30 minute incubation at 37° C. Following assembly, cells were transfer to a 96 well plate for fluorescence imaging using the EVOS system. To further test the robustness of the cell assembly, assembled cells were incubated 24 hours, followed by three times of centrifugation at 200 g for 5 minutes. Cell assembly were then assessed again by EVOS system.

Statistics and Reproducibility: Quantitative data were displayed as means±standard deviation (s.d.). Statistical significances were determined using one-way analysis of variance (ANOVA) with post-hoc Tukey's Test. All cell experiments were repeated independently at least three times.

### Results and Discussion

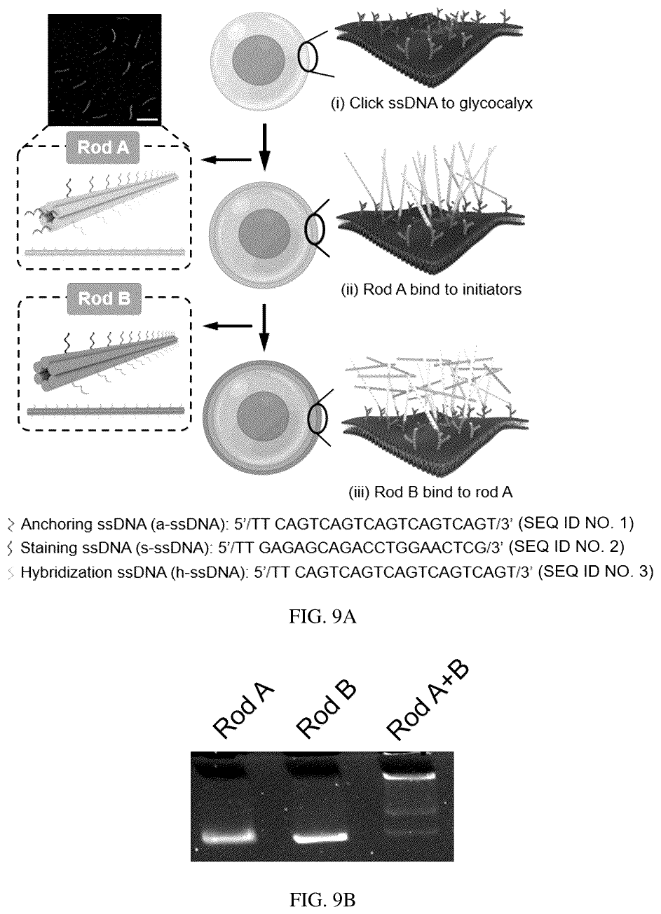

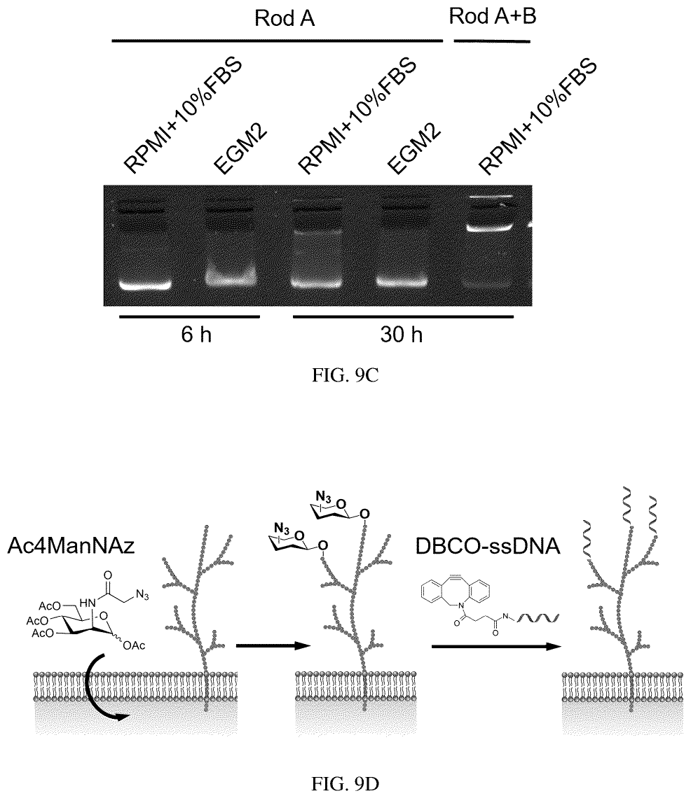

The nanoshell is designed to include of two layers of crosslinking DNA nanorods that are referred to herein in the Examples as “Rod A” and “Rod B”. Rod A and Rod B were both approximately 7 nanometers (“nm”) in diameter and approximately 400 nm in length. The rods were decorated with multiple functional single-stranded DNA (“ssDNA”) binding overhangs (FIG. 9A).

Rod A had three anchoring ssDNA (a-ssDNA) for allowing the anchoring of Rod A to membrane glycocalyx-anchored a-ssDNA complementary initiators (a′-ssDNA), 14 uniformly distributed staining ssDNA (s-ssDNA) for biotin attachment and subsequent streptavidin- fluorophore staining, and 14 uniformly distributed hybridization ssDNA (h-ssDNA) for the cross binding of Rod A and Rod B through ssDNA hybridization.

Rod B nanostructures had 14 s-ssDNA and 14 hybridization ssDNA complementary (h′-ssDNA) that allowed them to bind to h-ssDNA-decorated Rod A. Rod A had monodispersed individual bands when Rod A was modified with 7 or 14 h-ssDNA, showing the formation of DNA origami rods. When modified with 21 or 28 h-ssDNA, Rod A formation was not clear. Therefore, a higher number of h-ssDNA on Rod A led to unstable rod formation.

An atomic force microscopy (AFM) image verified the formation of the rods, which were constructed as six-helical bundles (FIG. 9A). Gel electrophoresis confirmed the monodispersity of the individual DNA origami rods. When combined, the formation of aggregate indicates the successful binding of the two rod species by hybridization (FIG. 9B). Two other distinct bands suggest that rod monomers and dimers existed at the same time. As the nanorods were to be used for cell culture applications, their stabilities in cell culture medium were evaluated (FIG. 9C). Individual rods had minimal degradation after 6 hours and 30 hours incubation at 37° C. in both cell culture medium. The aggregate, formed by the combination of Rod A and Rod B, did not degrade noticeably for up to 30 hours of incubation as well.

To anchor Rod A to the plasma membrane, using Jurkat cells as a suspended mammalian cell model, a method to first immobilize a′-ssDNA initiators onto the cell-surface glycocalyx was utilized (FIG. 9D) (Wijesekara et al., “Accessing and Assessing the Cell-Surface Glycocalyx Using DNA Origami”, Nano Lett. 2021,21: 4765-4773). In this method, azide ligands were covalently incorporated onto glycocalyx through metabolic glycan labeling using an azido monosaccharide, N-azidoacetylmannosamine-tetraacylated (Ac4ManNAz). a′-ssDNA were conjugated with dibenzocyclooctyne (DBCO) to form DBCO-a′-ssDNA through an N-hydroxysuccinimide ester (NHS-Ester) and amine reaction.

Bioorthogonal glycocalyx labeling with copper-free click chemistry allowed the conjugation of azide ligands on glycocalyx and DBCO-a′-ssDNA, leading to the immobilization of a′-ssDNA on glycocalyx. For these studies, Rod A was first introduced for glycocalyx binding, followed by Rod B for hybridization to immobilized Rod A.

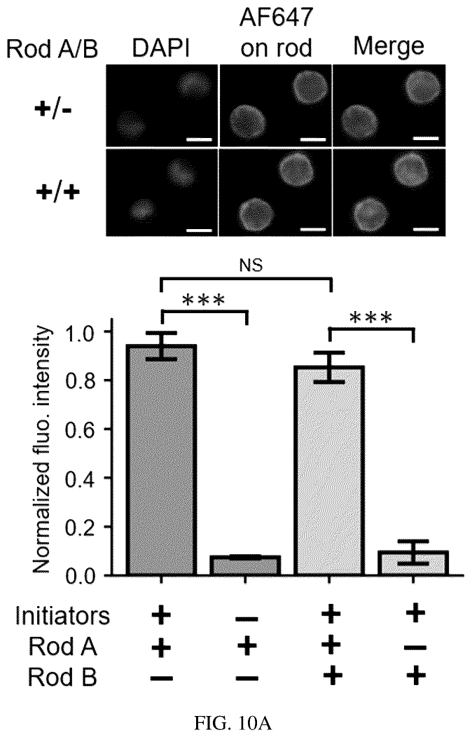

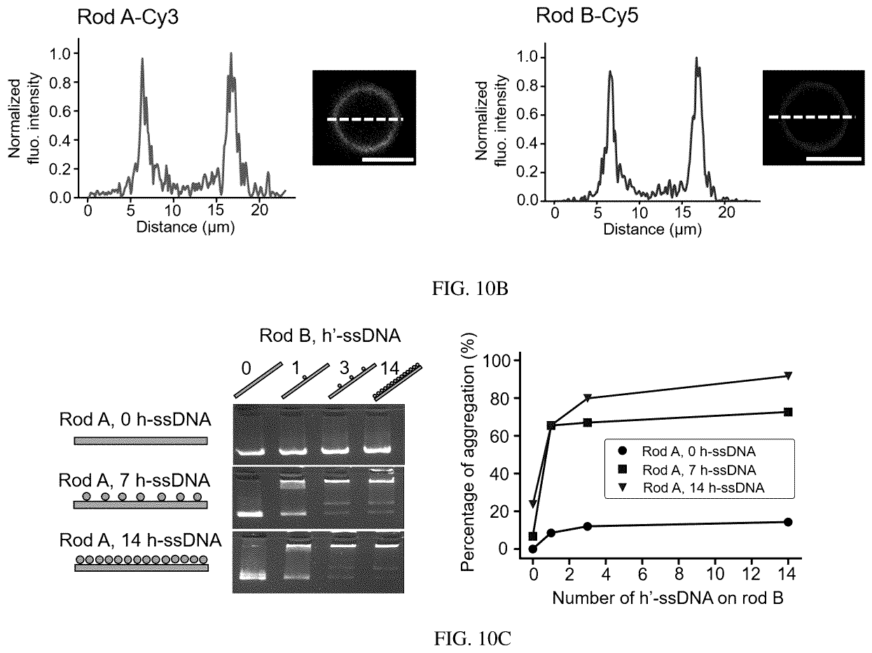

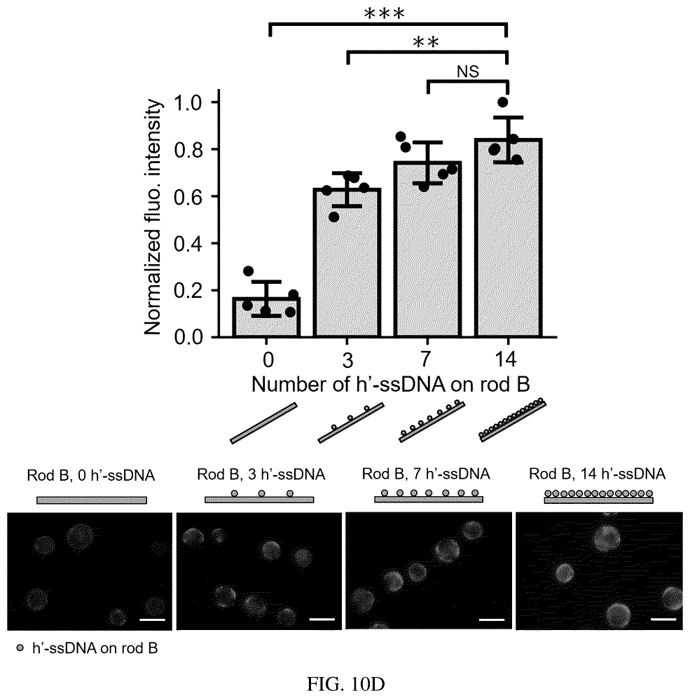

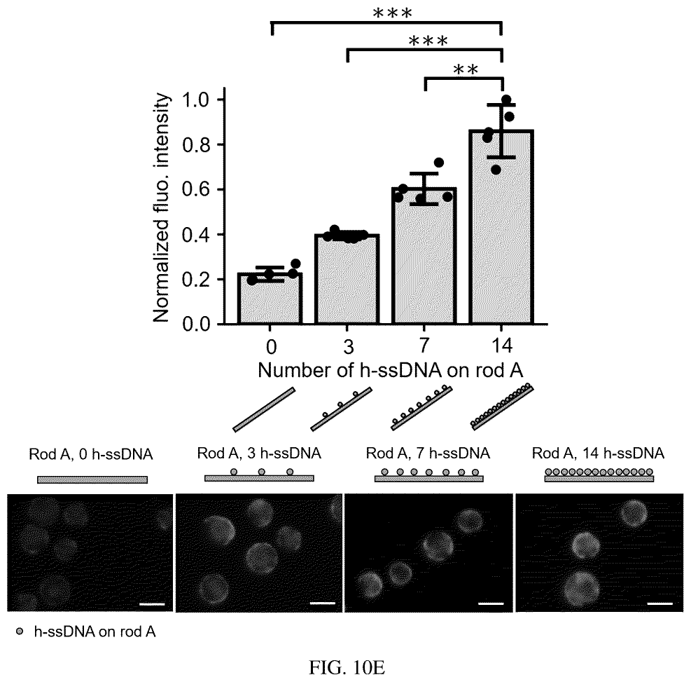

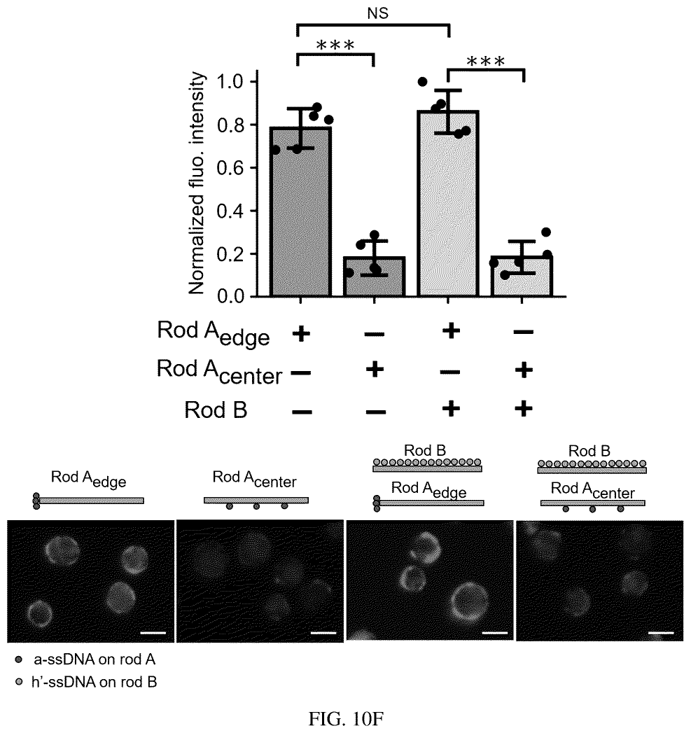

The successful recruitment of Rod A to the membrane and Rod B to Rod A was observed using fluorescence microscopy (FIG. 10A). Confocal microscopy cross-sectional images of cells coated with both rods and fluorescence intensity profiles extracted from those images further confirmed the binding of both Rod A and Rod B to the cell membrane and the formation of a nanoshell structure (FIG. 10B). To confirm the efficacy of this approach on more than one mammalian cell type, the synthesis strategy was replicated on HUVECs, demonstrating the versatility and utility of this nanoshell encapsulation technique for both non-adherent and adherent cell types. As the glycocalyx is on the surface of almost every mammalian cell, this glycocalyx-targeting method could be applicable to a broad array of cell types (Reitsma et al., “The endothelial glycocalyx: composition, functions, and visualization”, Pflugers Arch—Eur J Physiol, 2007, 454: 345-359; Weinbaum et al. “The Structure and Function of the Endothelial Glycocalyx Laye,” Annu. Rev. Biomed. Eng., 2007, 9: 121-167).

To demonstrate the ability to engineer the nanoshell, the roles of the functional ssDNA binding overhangs extending from the DNA nanorods were probed to investigate how the multivalency and positions of overhangs modulate the amount of rod binding in the nanoshell.

First, gel electrophoresis studies were performed by mixing DNA Rod A and Rod B with varying number of h-ssDNA and h′-ssDNA in suspension and incubating for 0.5 hours at 37° C. to allow for binding. The number of evenly displayed h-ssDNA on Rod A was 0, 7, or 14, and the number of h′-ssDNA on Rod B was from 0, 1, 3, or 14. Gel electrophoresis images and the quantification showed a monotonic decline of single rod band intensity and a monotonic increase in the aggregate band intensity with increasing number of h-ssDNA and h′-ssDNA (FIG. 10C). To confirm this finding on Jurkat cells, cells were first labeled with Rod A bearing 14 h-ssDNA overhangs. Next, fluorescently labeled Rod B with 0, 3, 7, or 14 h′-ssDNA were introduced. It was found that the binding of Rod B increased monotonically again with increasing number of h′-ssDNA on Rod B (FIG. 10D). In addition, fluorescently labeled Rod B with 14 h′-ssDNA were introduced to bind to rod A with 0, 3, 7, or 14 h-ssDNA. A similar trend of an increasing amount of Rod B binding with increasing valency of h-ssDNA on Rod A was observed (FIG. 10E). Furthermore, the binding of rods was also increased by adding higher concentrations of rods (FIGS. 11A-11B).

While determining that the multivalency of hybridization ssDNA on Rod A and Rod B regulated the recruitment of Rod B onto Rod A, it was found that the position of anchoring ssDNA on Rod A was important for the recruitment of both rods onto cell membranes. Rod A were modified to display a-ssDNA in two configurations: (1) at the edge and (2) at the center (FIG. 10F). The binding of edge-decorated Rod A to the glycocalyx and subsequent binding of Rod B to Rod A were significantly more than that of center-decorated Rod A. This finding was consistent with previous studies, stating that the recruitment of DNA nanostructures presenting ssDNA overhangs at the sharp or “pointy” areas is more efficient (Ding et al. “DNA Nanostructure-Programmed Like-Charge Attraction at the Cell-Membrane Interface”, ACS Cent. Sci., 2018, 4: 1344-1351; Wang et al. “Visualization of the Cellular Uptake and Trafficking of DNA Origami Nanostructures in Cancer Cells”, J. Am. Chem. Soc., 2018, 140: 2478-2484; Liu et al. “The effects of overhang placement and multivalency on cell labeling by DNA origami”, Nanoscale, 2021, 13: 6819-6828). These results demonstrate the ability to modulate the amount of nanorods incorporated into the nanoshell by changing the valency and positioning of functional ssDNA overhangs on both rods.

As the maximum thickness of the nanoshell is defined by the length of Rod A, an increase in rod binding indicates a higher density of rods. As a result of these findings, all the following studies were performed with three edge-located a-ssDNA and 14 side-located h-ssDNA on Rod A, and 14 side-located h′-ssDNA on Rod B.

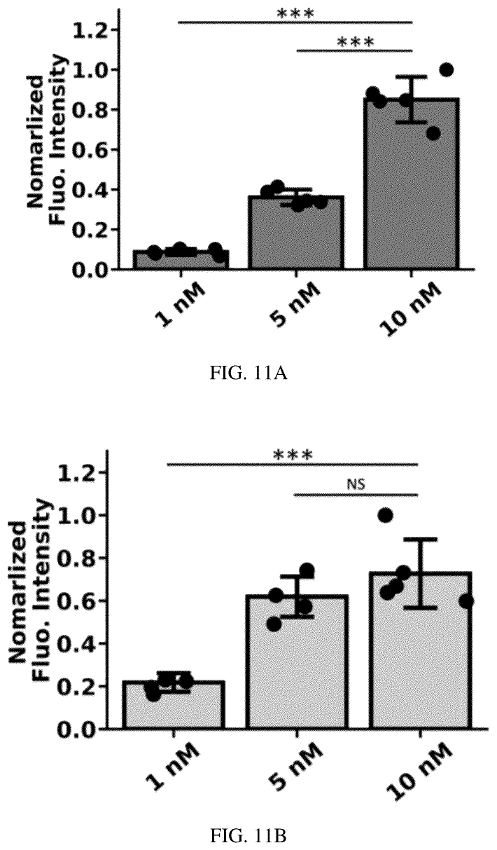

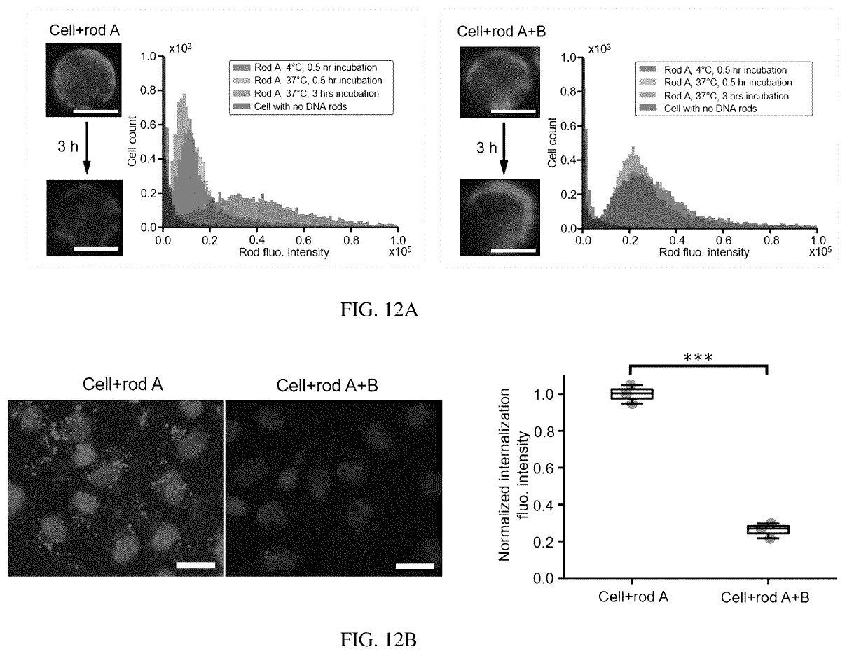

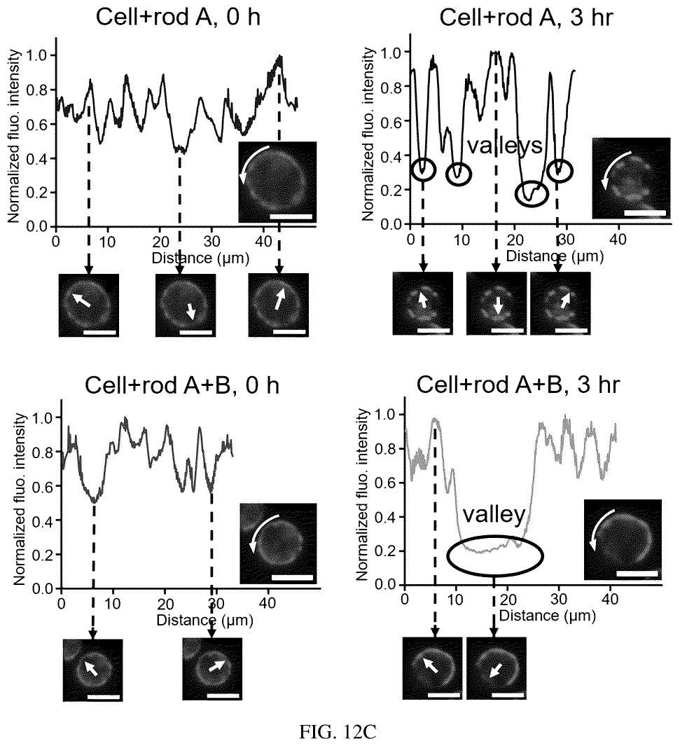

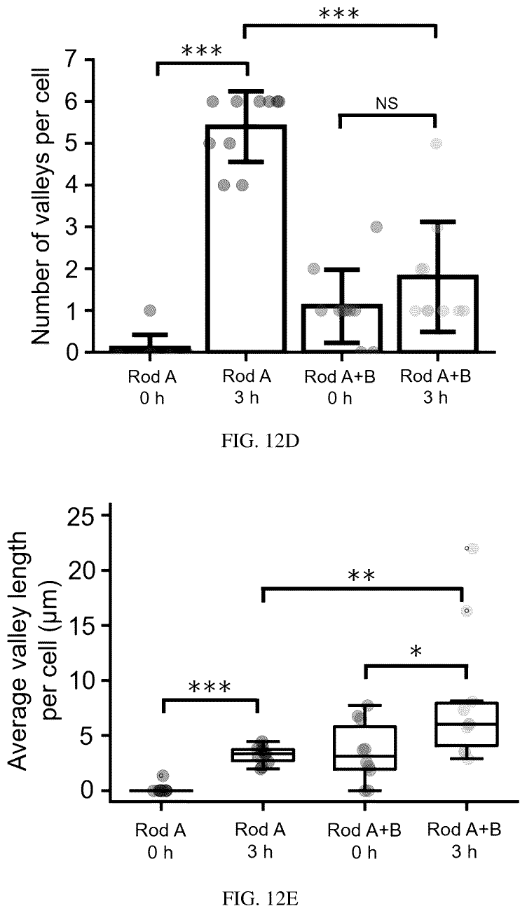

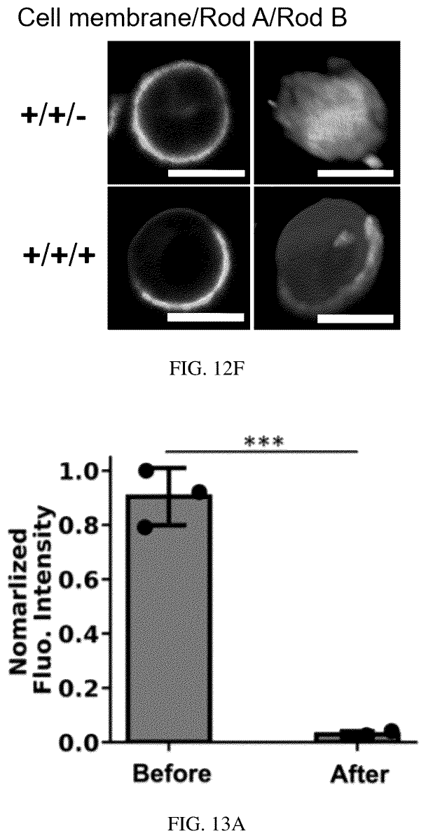

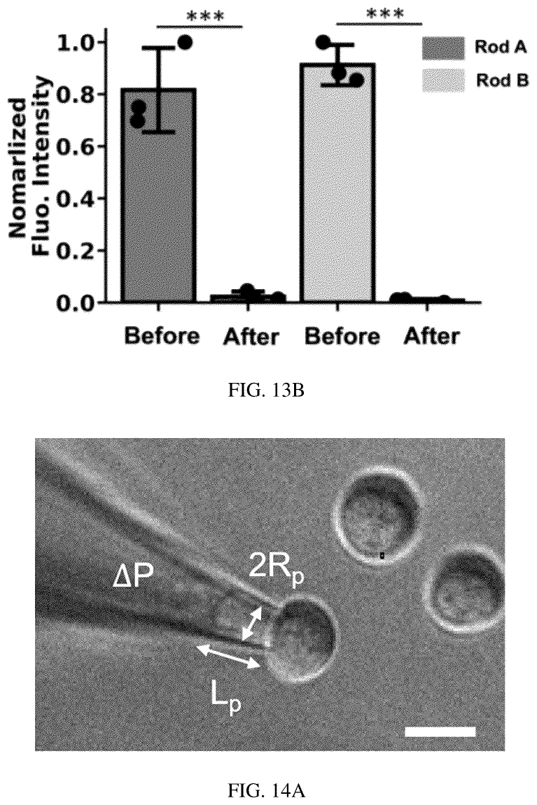

Cells constantly internalize substances outside the membrane, inducing membrane remodeling and deformations at multiple scales (Behzadi et al. “Cellular uptake of nanoparticles: journey inside the cell”, Chem. Soc. Rev., 2017, 46: 4218-4244; McMahon et al. “Membrane curvature and mechanisms of dynamic cell membrane remodeling”, Nature, 2005, 438: 590-596). It was therefore important to evaluate the stability of the nanoshell on the membrane. The surface retention time of the nanorods on the cell membrane was first evaluated as an indicator of stability. Nanoshell-coated cells and Rod A-coated cells were incubated under three conditions: (i) 4° C. for 0.5 hours; (ii) 37° C. for 0.5 hours; and (iii) 37° C. for 3 hours. The first incubation condition was regarded as a baseline as membrane movements and cell activities, especially cellular uptake, which lead to the destabilization of the nanoshells, were minimal. For consistent comparison, only biotinylated-Rod A were stained with streptavidin-AF647 and they were stained after incubation. This staining method allowed for the quantification of only the rods remaining on the external cell surface. From fluorescence-activated cell sorting (FACS) data, rod fluorescence intensity had a dramatic drop at 0.5 hours incubation at 37° C. as compared to 4° C., suggesting that single DNA rods had low stability after being anchored on cell membrane under physiological conditions (FIG. 12A). The fluorescence signal continued to decrease with further incubation at 37° C. to 3 hours, though at a slower rate. In contrast, on nanoshell-coated cells, we observed only a minimal decrease in fluorescence signal intensity with incubation at 37° C., even after 3 hours (FIG. 12A). Next, an examination on the cellular uptake of Rod A for Rod A-coated cells and nanoshell-coated cells using HUVECs was performed. It was found that there was more internalization of Rod A on Rod A-coated cells compared to that of nanoshell-coated cells (FIG. 12B). The improved surface retention time and decreased cellular uptake of rods showed that the crosslinking nanoshell had a higher stability and remained on the cell membrane for a longer duration, as compared to single rod attachment without crosslinking. Moreover, although having a high stability under physiological conditions, the nanoshell can still be degraded through the simple administration of DNase I, making temporary encapsulation possible (FIGS. 13A-13B).

The substantial remodeling of nanoshell was observed as the pattern of rod fluorescence signal evolved throughout incubation period (cell fluorescence images in FIG. 12A). The distribution of fluorescence signals was tracked in cell images from these studies and the signal intensity around the contour of cell borders was measured (FIG. 12C). At the 0 hour time point, where the cellular uptake and membrane detachment of rods were minimal due to insufficient incubation time, relatively continuous and uniform intensity was observed in Rod A-coated cells.

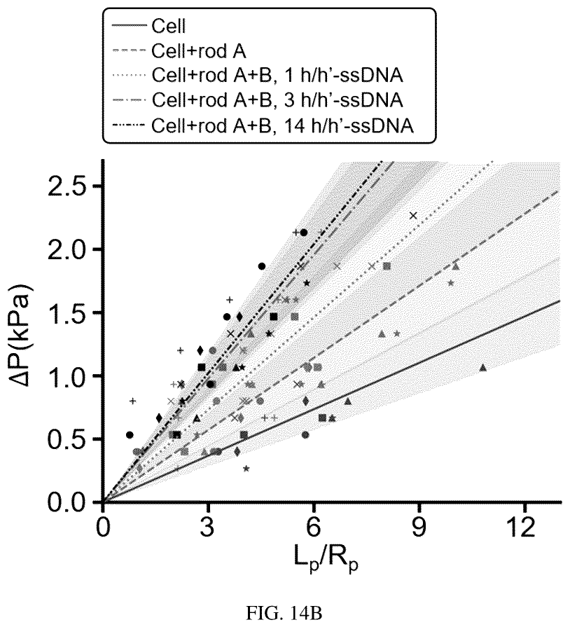

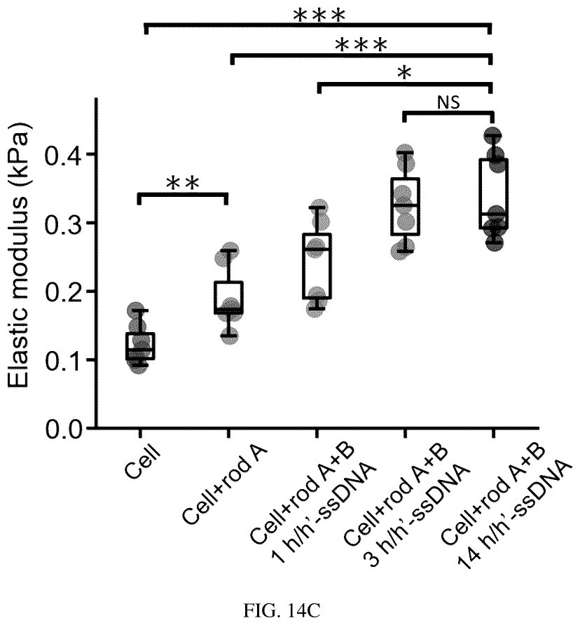

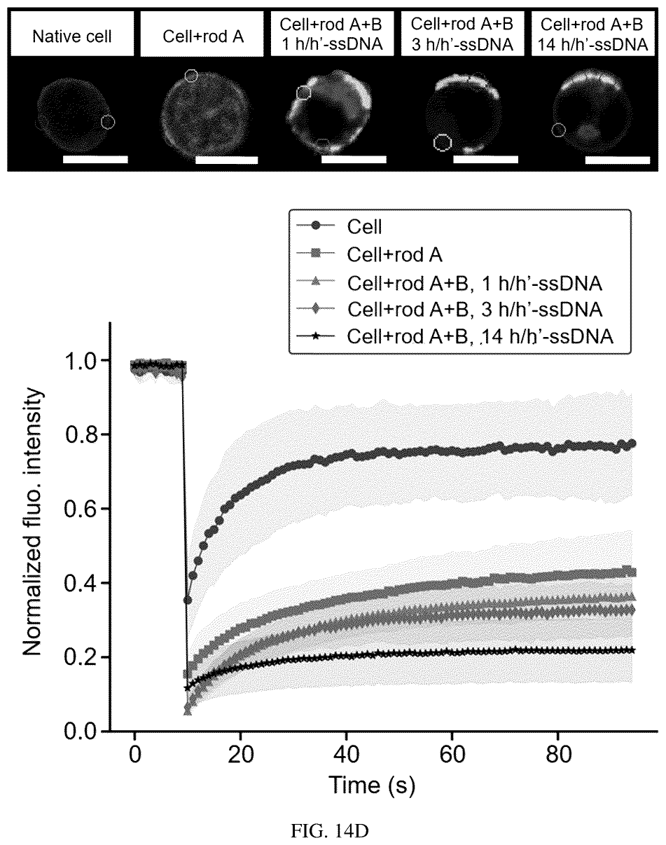

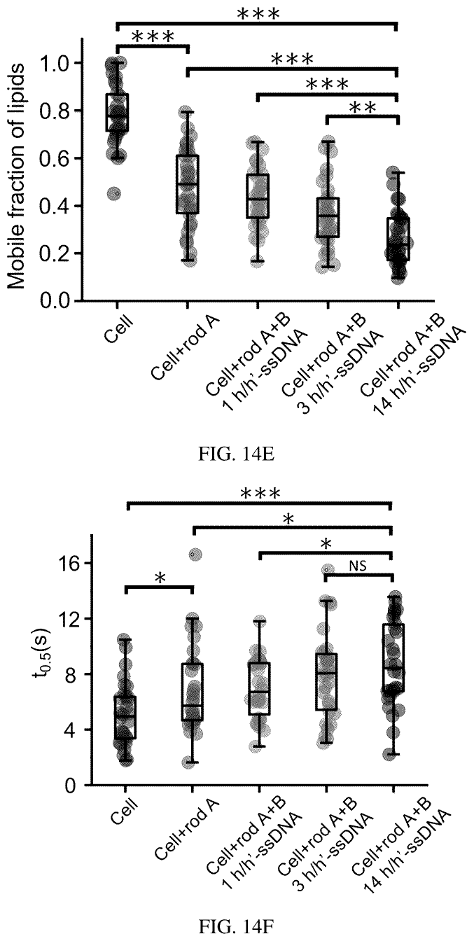

However, the signal became discretized and non-uniform after 3 hours incubation with signal intensity disappearing in discrete regions on cell borders, which resulted in signal valleys, suggesting long-term incubation destabilized the membrane-anchored DNA rods. Signal valleys also appeared in nanoshell-coated cells but were fewer in number and were substantially wider, potentially due to the crosslinking and polymerization of rods. The number and the length of signal valleys were then quantified (FIGS. 12D and 12E). Data showed that the discretization of fluorescence signals was dependent on two factors: (1) incubation time; and (2) the addition of crosslinking Rod B. Incubation-induced signal valleys were presumably due to cellular uptake whereas crosslinking-induced valleys suggested that the rods remodeled their positions on the cell membrane during crosslinking process and incubation. A similar phenomenon was also reported previously on GUVs where the distribution of DNA origami and the morphology of lipid bilayers were altered after the triggered polymerization of DNA origami (Franquelim et al. “Membrane sculpting by curved DNA origami scaffolds. Nat Commun, 2018, 9: 811; Journot et al. “Modifying Membrane Morphology and Interactions with DNA Origami Clathrin-Mimic Networks”, ACS Nano, 2019,13: 9973-9979). Rod A-coated cells and nanoshell-coated cells were imaged with confocal microscopy and their three-dimensional models were reconstructed. The images revealed uniform covering on Rod A-coated cells and partial, localized coverage on nanoshell-coated cells (FIG. 12F). This showed the role of the dynamic interactions between the DNA rods and the cell membrane in repositioning membrane-anchored substances. Membrane deformations were not observed due to DNA construct polymerization (Czogalla et al. “Amphipathic DNA Origami Nanoparticles to Scaffold and Deform Lipid Membrane Vesicles”, Angew. Chem. Int. Ed., 2015, 54:6501-6505; Franquelim et al.; Journot et al.). After demonstrating the ability of the nanoshell to remodel and stabilize itself, the affect of this stabilization on the biophysical properties of the cell membrane, with a focus on membrane stiffness and lipid fluidity was investigated. First, membrane elastic modulus was evaluated by performing micropipette aspiration on pre-treatment native cells and nanoshell-coated cells (FIG. 14A). Cells (having a radius (R) of the cell of approximately 5 μm) were aspirated into micropipettes (having a radius (Rₚ) of 2.5 μm) through aspiration pressure change (ΔP). The elastic modulus (E) can be derived from Equation 1, assuming the cell as a continuum-medium model with homogeneity,

$$\frac{Δ P}{E} = \frac{2 π}{3 {ϕ}_{p}} \frac{{L}_{p}}{{R}_{p}} \qquad ( Equation 1 )$$

where Lp is the aspiration length and ϕₚ is approximately 2.1. The membrane elastic modulus of nanoshell-coated cells was 0.340±0.062 kPa (mean±s.d.), which was around 3-fold that of native cells, measured to be 0.122±0.029 kPa (FIGS. 14B and 14C). A gradual increase in the elastic modulus with an increasing valency of h/h′-ssDNA was also observed, showing the tunability of the membrane stiffening. These results indicated that the nanoshell formed by crosslinking rods mechanically supported the membrane and enhanced the membrane mechanics, functioning analogously to the cytoskeleton underneath the membrane, which is consistent with the crosslinking of the stiff 6-helix nanorod constructs.

A previous study found that by decorating DNA origami on GUVs with cholesterol anchors, the fluidity of artificial lipid was not affected (Czogalla et al. “Switchable domain partitioning and diffusion of DNA origami rods on membranes. Faraday Discuss. 2013, 161, 31-43). To address this question, fluorescence recovery after photobleaching (FRAP) experiments were performed to evaluate the mobile fraction of membrane lipid and the rate of recovery (FIG. 14D). Interestingly, it was observed that the glycocalyx-anchored nanoshell greatly reduced lipid mobility (FIG. 14E). Specifically, in native cells, fluorescence signal recovered to approximately 80% of the pre-photobleaching level within 30 seconds. However, for Rod A-coated cells, the recovery dropped to approximately 40%. The number further decreased when two-component nanoshell was applied and when the valency of h/h′-ssDNA increased. The recovery here represents the mobile fraction of membrane lipid. It was also noted that the rate of recovery was much slower after cells were coated with DNA rods (FIG. 14F). The half time recovery of nanoshell-coated cell membrane lipid was only around half of that of native cell membrane lipid. These results demonstrated that a certain amount of membrane lipid experienced gelation when the membrane was coated with DNA rods and nanoshell, especially the latter case, resulting in a significant decrease in membrane lipid mobility. Consistent with tunable membrane stiffening, the lipid fluidity also demonstrated tenability by altering the valency of overhangs, as more h/h′-ssDNA resulted in more rod crosslinking (FIGS. 14D-14F). The membrane-anchored DNA rods also lost their mobility whereas by comparison, DBCO-cy5 had nearly the same mobility as membrane lipid (FIGS. 15A and 15B). Taken together, these observations suggested that reductions in lipid mobility are not due to the metabolic glycan labeling nor the click conjugation of azide ligands and DBCO molecules, but rather, due to the recruitment of DNA origami. Their large molecular weights and the potential spatial hinderance may be responsible for the low mobility of DNA origami. These findings showed that the crosslinking of DNA rods further decreased the fluidity of membrane lipid, presumably due to enhanced rod-membrane interactions induced by rod crosslinking.

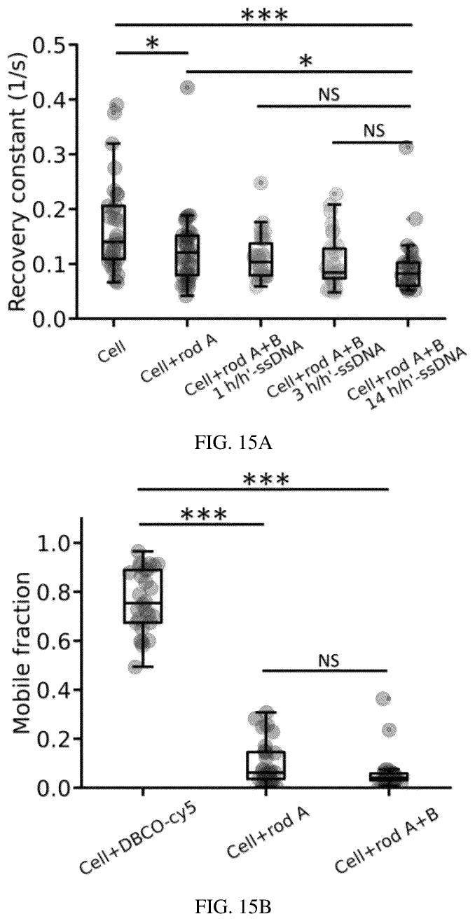

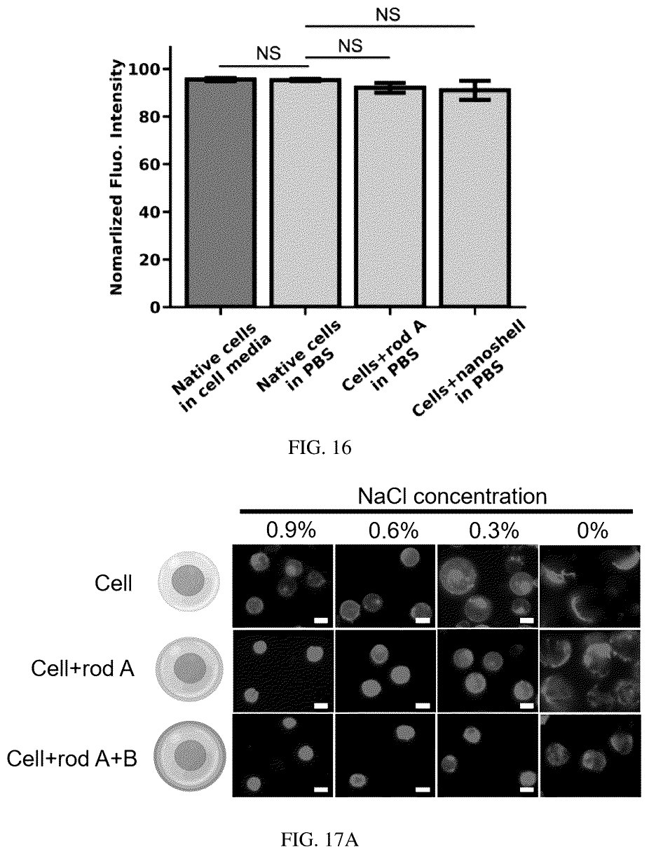

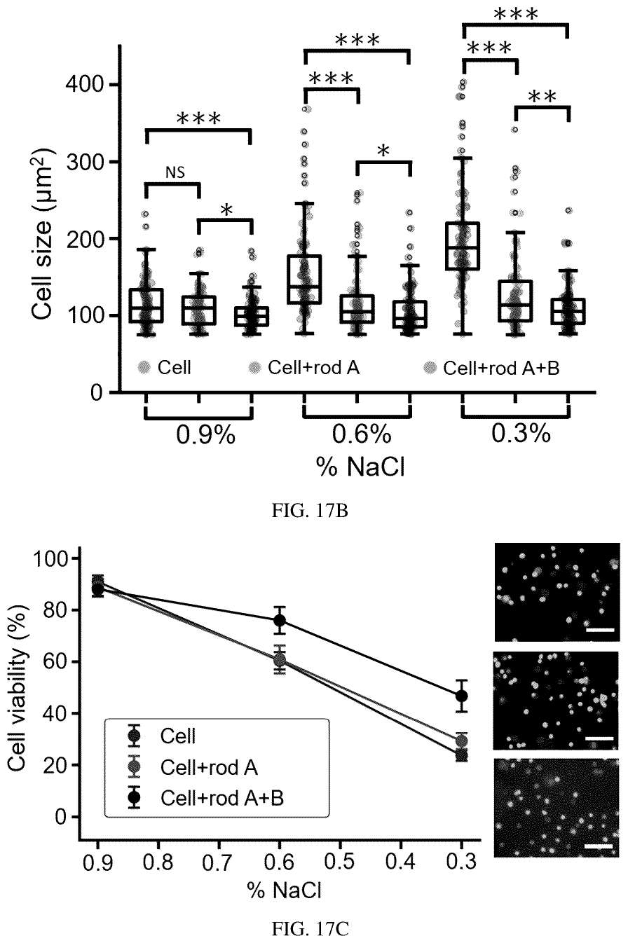

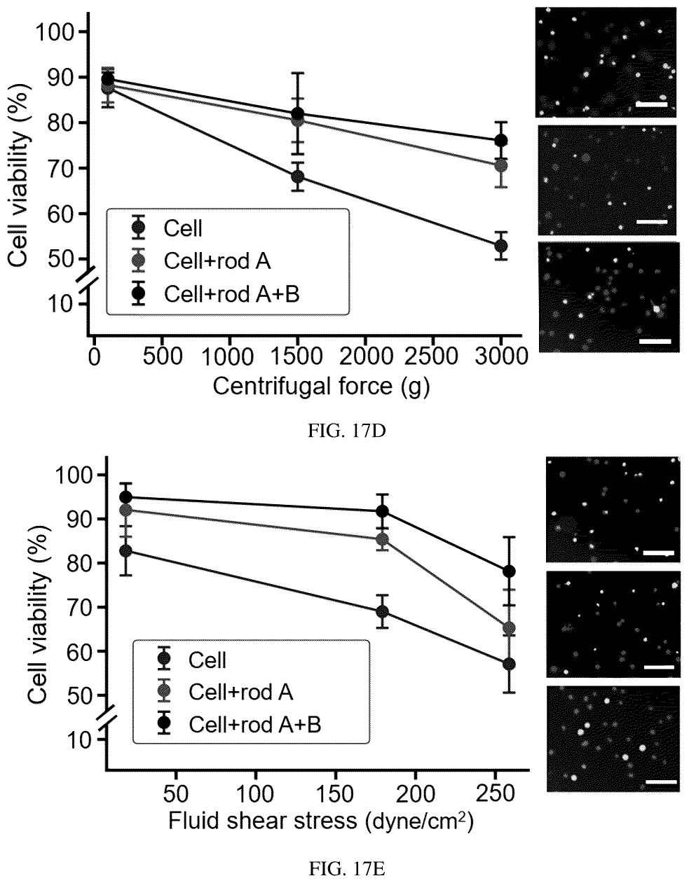

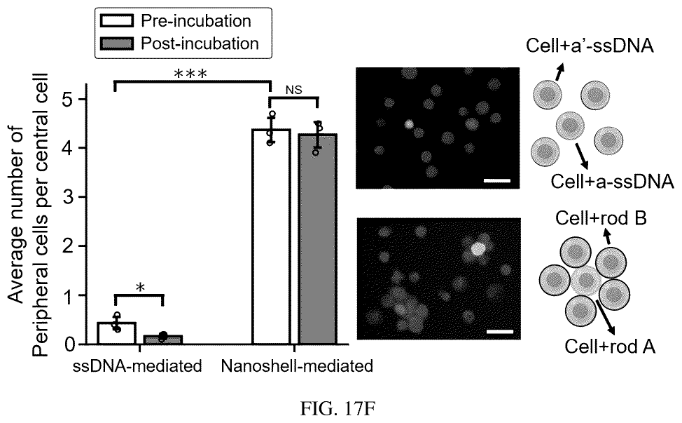

The cell membrane is vital for maintaining cell size, shape and integrity, protecting the cell from outside assaults. The enhancement in membrane mechanics and the gelation of certain membrane lipid due to the nanoshell coating showed the potential of the nanoshell in providing protection to cells under harsh and mechanically challenging environments. The cell viability after cells were coated with nanoshell were examined as a baseline. As expected, nanoshell-coated cells had high viability as the DNA nanostructures were biocompatible and the whole synthesis process was performed under physiological conditions (FIG. 16). Osmotic imbalanced solutions were then applied to cells by changing the sodium chloride (NaCl) concentration from 0.9% to 0.6%, 0.3% and 0%. The resulting cell sizes and viability were measured (FIG. 17A-17C). As the osmotic pressure decreased, a rapid increase in the sizes of native cells and a decrease in their viability were found, whereas nanoshell-coated cells maintained the cell size and had an approximately 20% higher cell viability in lower osmotic solutions, with statistical differences compared to pre-treatment native cells (FIGS. 17B and 17C). Notably, the nanoshell systems even maintained cell shape under 0% NaCl after 10 min incubation. In contrast, native cells and rod A-coated cells burst rapidly within seconds. Although the Rod A coating was also able to limit cell expansion, it was not able rescue cell viability in low NaCl concentration solutions. A slight decrease in the baseline cell size was noticed under 0.9% NaCl with 115±30 μm² for native cells, 109±24 μm² for Rod A-coated cells and 102±20 μm² for nanoshell-coated cells, with statistical difference between nanoshell-coated cells and Rod A-coated cells. These findings demonstrated the utility of coating DNA origami on the membrane to maintain cell size. Even a simple coating of Rod A can limit cell expansion and improve survival, but the crosslinked nanoshell with both Rods A and B was the most effective in limiting expansion and acting as an armor and improving the survival of cells.

Centrifugal forces were applied to cells and it was found that the nanoshell coating was able to rescue viability under 1500 g and 3000 g (FIG. 17D) (Shi et al. “DNA-templated synthesis of biomimetic cell wall for nanoencapsulation and protection of mammalian cells”, Nat Commun., 2019, 10, 2223). The Rod A coating was also able to improve cell viability against centrifugation, similar to its ability in limiting expansion under osmotic swelling.

Furthermore, using a syringe pump system to eject cells, the cell viability under various fluid shear stresses was examined (Barnes et al. “Resistance to Fluid Shear Stress Is a Conserved Biophysical Property of Malignant Cells”, PLoS ONE, 2012, 7, e50973). By changing the flow rate, 18, 179 and 259 dyne/cm 2 of shear stress were applied to cells passing through the needle, according to Poiseuille's equation (Equation 2),

$$τ = \frac{4 Q η}{π {R}^{3}} \qquad ( Equation 2 )$$

where τ is the fluid shear stress, Q is flow rate in cubic centimeters per second (cm³/s), η is the dynamic viscosity of cell medium, which is treated as water (0.01 dyne second per square centimeters (dyne·s/cm²)), and R is the radius of the needle. The syringe ejection process was repeated 10 times. The findings revealed that nanoshell-coated cells exhibit higher viability across all three shear stress conditions, suggesting that the nanoshell helps the cell resist against fluid shears (FIG. 17E). Compared to native cells, nanoshell-coated cells showed a substantial increase in cell viability, rescuing up to 20% more cells.

The mechanism of nanoshell formation through crosslinking can also be applied to facilitate multicellular assembly. Two groups of cells were immobilized with cell-surface anchors using same concentration of cholesterol-a-ssDNA and cholesterol-a′-ssDNA, respectively. Subsequently, Rod A and Rod B (both having three a-ssDNA at edge) were recruited onto the cell membranes. Comparing cell assembly mediated solely by ssDNA (using cells with only a/a′-ssDNA) and nanoshell-mediated assembly, where Rod A and Rod B crosslinked with each other to form assemblies, it was found that the nanoshell-mediated assembly exhibited a significantly higher number of peripheral cells compared to the ssDNA-mediated assembly (FIG. 17F). Furthermore, the effectiveness of the nanoshell-mediated assembly was maintained even after 24 hours of incubation followed by centrifugation, whereas the ssDNA-mediated assemblies were less robust.

These results demonstrated that the DNA origami nanoshell protects cells in challenging environments and its potential benefits for bioengineering applications such as cell printing and multicellular assembly.

It will be readily appreciated by those skilled in the art that modifications may be made to the invention without departing from the concepts disclosed in the foregoing description. Accordingly, the particular embodiments described in detail herein are illustrative only and are not limiting to the scope of the invention, which is to be given the full breadth of the appended claims and any and all equivalents thereof.

The invention claimed is:

1. A ruggedized particle or cell comprising: a particle or live cell; and
a shell comprising a plurality of first nucleic acid and/or nucleic acid analog nanostructures anchored to the particle or cell; and a plurality of second nucleic acid and/or nucleic acid analog nanostructures cross-linking the plurality of first nucleic acid and/or nucleic acid analog nanostructures by a plurality of nucleic acid and/or nucleic acid analog staples.

2. The ruggedized particle or cell of claim 1, wherein the particle or live cell is a live cell.

3. The ruggedized particle or cell of claim 2, comprising:
the live cell;
a plurality of first nucleic acid and/or nucleic acid analog nanostructure beams anchored to the cell, the beams comprising one or more single-stranded scaffold nucleic acids or nucleic acid analogs;
nucleic acids and/or nucleic acid analog staples attached to the plurality of first nucleic acid and/or nucleic acid analog nanostructure beams and nucleic acids and/or nucleic acid analog staples attached to the plurality of second nucleic acid and/or nucleic acid analog nanostructures, wherein the nucleic acids and/or nucleic acid analog staples attached to the plurality of second nucleic acid and/or nucleic acid analog nanostructures are complementary to the nucleic acids and/or nucleic acid analog staples attached to the plurality of first nucleic acid and/or nucleic acid analog nanostructure beams; and
the plurality of second nucleic acid and/or nucleic acid analog nanostructures linked to the plurality of first nucleic acid and/or nucleic acid analog nanostructure beams by the staples and which cross-link two or more of the first nucleic acid and/or nucleic acid analog nanostructures to the second nucleic acid and/or nucleic acid analog nanostructures.

4. The ruggedized particle or cell of claim 2, wherein the plurality of first nucleic acid and/or nucleic acid analog nanostructures are anchored to the glycocalyx of the cell.

5. The ruggedized particle or cell of claim 2, wherein the plurality of first nucleic acid and/or nucleic acid analog nanostructures are anchored to a surface protein of the cell, such as a receptor.

6. The ruggedized particle or cell of claim 2, wherein the plurality of first nucleic acid and/or nucleic acid analog nanostructures are anchored to the surface protein of the cell or to the glycocalyx of the cell by a click chemistry reaction, and optionally a biorthogonal click chemistry method.

7. The ruggedized particle or cell of claim 2, wherein the plurality of first nucleic acid and/or nucleic acid analog nanostructures are linked to a hydrophilic moiety and are anchored to the lipid bilayer membrane of the cell via the hydrophobic moiety.

8. The ruggedized particle or cell of claim 7, wherein the hydrophobic moiety comprises one or more fatty acid or cholesterol moieties.

9. The ruggedized particle or cell of claim 7, wherein the hydrophobic moiety comprises a glycosylphosphatidylinositol (GPI) moiety.

10. The ruggedized particle or cell of claim 2, wherein the cell is a multipotent cell or a pluripotent cell such as a stem cell, e.g., a mesenchymal stem cell, a cell expressing a recombinant protein or RNA, an immune system cell, such as an antigen-presenting cell, a lymphocyte, a neutrophil, or a monocyte, or a platelet.

11. The ruggedized particle or cell of claim 1, wherein the particle or live cell is a lipid-based nanoparticle, a lipid-based nanocarrier, an inorganic nanoparticle, or a polymeric nanoparticle.

12. The ruggedized particle or cell of claim 11, wherein the particle or live cell is a nanoemulsion, a solid lipid nanoparticle, a phospholipid micelle, a liposome, a vesicle, an exosome, a giant unilamellar vesicle, an iron oxide nanoparticle, a gold nanoparticle, a carbon allotrope particle, a PLGA (poly(lactic-co-glycolic acid)) particle, an m PEG-PGA (methoxy-poly(ethyleneglycol)-block-poly(L-glutamic acid)) particle; a PEG-PMT (PEG-poly(ω-penta-decalactone-co-N-methyldiethyleneamine sebacate-co-2,2′-thiodiethylene sebacate) particle, or a PEI-PLGA (polyethyleneimine-PLGA) particle.

13. The ruggedized particle or cell of claim 1, wherein the plurality of second nucleic acid and/or nucleic acid analog nanostructures comprise nucleic acid and/or nucleic acid analog nanostructure beams.

14. The ruggedized particle or cell claim 13, wherein the beams of the first nucleic acid and/or nucleic acid analog nanostructures are comprise from two to ten rods (e.g., helical nucleic acid and/or nucleic acid analog bundles), such as six rods and the beams of the second nucleic acid and/or nucleic acid analog nanostructures optionally comprise from two to ten rods, such as six rods.

15. The ruggedized particle or cell of claim 14, wherein the rods of the first nucleic acid and/or nucleic acid analog nanostructures are anchored at an end to the cell.

16. The ruggedized particle or cell of claim 1, further comprising a nucleic acid and/or nucleic acid analog nanostructure environmental sensor attached to the shell, wherein the environmental sensor optionally comprises a fluorophore or a FRET pair.

17. A method of preparing a ruggedized particle or cell, comprising anchoring a plurality of first nucleic acid and/or nucleic acid analog nanostructures to a surface of a particle or a live cell, and forming a shell by crosslinking the plurality of first nucleic acid and/or nucleic acid analog nanostructures with a plurality of second nucleic acid and/or nucleic acid analog nanostructures using a plurality of nucleic acids and/or nucleic acid analog staples, wherein the plurality of first nucleic acid and/or nucleic acid analog nanostructures are attached to the plurality of second nucleic acid and/or nucleic acid analog nanostructures.

18. The method of claim 17, wherein the particle or cell is a live cell.

19. A method of preparing a graft for tissue replacement or repair in a patient, the method comprising:
depositing a ruggedized cell as claimed in claim 2 onto the surface of a polymer matrix structure;
culturing the ruggedized cells on the polymer matrix structure under conditions for differentiating the cells into cells of the tissue to be repaired or replaced; and
culturing the ruggedized cells to expand the number of cells on the polymer matrix structure.

20. An environmental sensing method comprising, introducing the ruggedized particle or cell of claim 16 to a sensing environment, and determining the effect of the environment on the environmental sensor.


SEQUENCE LISTING (181 sequences)

SEQ ID NO: 1 - DNA, 22 bp, synthetic construct
  source 1..22: mol_type = other DNA
ttcagtcagtcagtcagtcagt

SEQ ID NO: 2 - DNA, 22 bp, synthetic construct
  source 1..22: mol_type = other DNA
ttgagagcagacctggaactcg

SEQ ID NO: 3 - DNA, 22 bp, synthetic construct
  source 1..22: mol_type = other DNA
ttgtgacataccttcggagcat

SEQ ID NO: 4 - DNA, 22 bp, synthetic construct
  source 1..22: mol_type = other DNA
ttatgctccgaaggtatgtcac

SEQ ID NO: 5 - DNA, 22 bp, synthetic construct
  source 1..22: mol_type = other DNA
ttcgagttccaggtctgctctc

SEQ ID NO: 6 - DNA, 22 bp, synthetic construct
  source 1..22: mol_type = other DNA
ttactgactgactgactgactg

SEQ ID NO: 7 - DNA, 20 bp, synthetic construct
  source 1..20: mol_type = other DNA
cagtcagtcagtcagtcagt

SEQ ID NO: 8 - DNA, 20 bp, synthetic construct
  source 1..20: mol_type = other DNA
actgactgactgactgactg

SEQ ID NO: 9 - DNA, 42 bp, synthetic construct
  source 1..42: mol_type = other DNA
tcaggctgcgcaacctagggcgctggcaatcgtctgaaatgg

SEQ ID NO: 10 - DNA, 42 bp, synthetic construct
  source 1..42: mol_type = other DNA
cataacgccaaaagttgctaaacaacttccaataggaaccca

SEQ ID NO: 11 - DNA, 42 bp, synthetic construct
  source 1..42: mol_type = other DNA
ccgcttctggtgccccacacccgccgcgacaggaaaaacgct

SEQ ID NO: 12 - DNA, 42 bp, synthetic construct
  source 1..42: mol_type = other DNA
tatcggcctcaggaatggttgctttgacttgctggtaatatc

SEQ ID NO: 13 - DNA, 42 bp, synthetic construct
  source 1..42: mol_type = other DNA
catcgtaaccgtgcgaatcagagcgggaataacatcacttgc

SEQ ID NO: 14 - DNA, 42 bp, synthetic construct
  source 1..42: mol_type = other DNA
gattgaccgtaatgttagacaggaacggtcacgcaaattaac

SEQ ID NO: 15 - DNA, 42 bp, synthetic construct
  source 1..42: mol_type = other DNA
tcagttgagatttaaaggaacaactaaaccaccctcagagcc

SEQ ID NO: 16 - DNA, 42 bp, synthetic construct
  source 1..42: mol_type = other DNA
aacgaactaacggatgaaaatctccaaaggtttagtaccgcc

SEQ ID NO: 17 - DNA, 42 bp, synthetic construct
  source 1..42: mol_type = other DNA
tataccagtcaggagtatcggtttatcaatataagtatagcc

SEQ ID NO: 18 - DNA, 42 bp, synthetic construct
  source 1..42: mol_type = other DNA
atcattgtgaattaagcttgataccgatttttgctcagtacc

SEQ ID NO: 19 - DNA, 42 bp, synthetic construct
  source 1..42: mol_type = other DNA
cgagtagtaaattggcccacgcataaccagaggctgagactc

SEQ ID NO: 20 - DNA, 42 bp, synthetic construct
  source 1..42: mol_type = other DNA
tcattcagtgaatagagttaaaggccgctgcctatttcggaa

SEQ ID NO: 21 - DNA, 42 bp, synthetic construct
  source 1..42: mol_type = other DNA
agaaccggatattcaaagacagcatcgggtgccttgagtaac

SEQ ID NO: 22 - DNA, 42 bp, synthetic construct
  source 1..42: mol_type = other DNA
ggcgcataggctggttgaggactaaagagatgatacaggagt

SEQ ID NO: 23 - DNA, 42 bp, synthetic construct
  source 1..42: mol_type = other DNA
tgaccaactttgaagggtaaaatacgtatctctgaatttacc

SEQ ID NO: 24 - DNA, 42 bp, synthetic construct
  source 1..42: mol_type = other DNA
gccggaacgaggcgcgaaagaggcaaaacaaacaaataaatc

SEQ ID NO: 25 - DNA, 42 bp, synthetic construct
  source 1..42: mol_type = other DNA
gataaattgtgtcgcccagcgattatacgaagtatgttgagg

SEQ ID NO: 26 - DNA, 42 bp, synthetic construct
  source 1..42: mol_type = other DNA
ttgcgtattgggcgcttttcaccagtgaaatagattagagcc

SEQ ID NO: 27 - DNA, 42 bp, synthetic construct
  source 1..42: mol_type = other DNA
tatcataaccctcgcgtctttccagacggtacaaactacaac

SEQ ID NO: 28 - DNA, 42 bp, synthetic construct
  source 1..42: mol_type = other DNA
cagctgcattaatggcctggccctgagatgaggaaggttatc

SEQ ID NO: 29 - DNA, 42 bp, synthetic construct
  source 1..42: mol_type = other DNA
ttgcgctcactgcctgccccagcaggcgatcaatatctggtc

SEQ ID NO: 30 - DNA, 42 bp, synthetic construct
  source 1..42: mol_type = other DNA
gcctggggtgcctatcggcaaaatcccttctaaagcatcacc

SEQ ID NO: 31 - DNA, 42 bp, synthetic construct
  source 1..42: mol_type = other DNA
acaattccacacaaggttgagtgttgttcctgcaacagtgcc

SEQ ID NO: 32 - DNA, 42 bp, synthetic construct
  source 1..42: mol_type = other DNA
tcatggtcatagctagaacgtggactccgcagaagataaaac

SEQ ID NO: 33 - DNA, 42 bp, synthetic construct
  source 1..42: mol_type = other DNA
tcgactctagaggaagggcgatggcccaagccctaaaacatc

SEQ ID NO: 34 - DNA, 42 bp, synthetic construct
  source 1..42: mol_type = other DNA
gttgtaaaacgacgttttggggtcgaggaatatttttgaatg

SEQ ID NO: 35 - DNA, 42 bp, synthetic construct
  source 1..42: mol_type = other DNA
ggcgattaagttggaagggagcccccgagaacccttctgacc

SEQ ID NO: 36 - DNA, 42 bp, synthetic construct
  source 1..42: mol_type = other DNA
cttcgctattacgcacgtggcgagaaagacacgaccagtaat

SEQ ID NO: 37 - DNA, 42 bp, synthetic construct
  source 1..42: mol_type = other DNA
gagaagtgtttttagtcggattctccgtaaatgtgagcgagt

SEQ ID NO: 38 - DNA, 42 bp, synthetic construct
  source 1..42: mol_type = other DNA
attatttacattggaattaattacatttcgttatacaaattc

SEQ ID NO: 39 - DNA, 42 bp, synthetic construct
  source 1..42: mol_type = other DNA
catggaaatacctaatggaaacagtacacggaatcataatta

SEQ ID NO: 40 - DNA, 42 bp, synthetic construct
  source 1..42: mol_type = other DNA
cagaacaatattactgcttctgtaaatcaccgaccgtgtgat

SEQ ID NO: 41 - DNA, 42 bp, synthetic construct
  source 1..42: mol_type = other DNA
ctgagtagaagaactccttgaaaacatatagttaatttcatc

SEQ ID NO: 42 - DNA, 42 bp, synthetic construct
  source 1..42: mol_type = other DNA
cgttgtagcaatacgagtcaatagtgaatcgcaagacaaaga

SEQ ID NO: 43 - DNA, 42 bp, synthetic construct
  source 1..42: mol_type = other DNA
gaggccaccgagtaacctttttaacctcgttgggttatataa

SEQ ID NO: 44 - DNA, 42 bp, synthetic construct
  source 1..42: mol_type = other DNA
accaccctcattttcaaagacaaaagggaacaaagttaccag

SEQ ID NO: 45 - DNA, 42 bp, synthetic construct
  source 1..42: mol_type = other DNA
accctcagaaccgcgtaaatattgacggatcttaccgaagcc

SEQ ID NO: 46 - DNA, 42 bp, synthetic construct
  source 1..42: mol_type = other DNA
cggaataggtgtatcgtcaccgacttgaagcccaataataag

SEQ ID NO: 47 - DNA, 42 bp, synthetic construct
  source 1..42: mol_type = other DNA
aggcggataagtgccaccagtagcaccatgagcgctaatatc

SEQ ID NO: 48 - DNA, 42 bp, synthetic construct
  source 1..42: mol_type = other DNA
ctcaagagaaggatcaatgaaaccatcggggagaattaactg

SEQ ID NO: 49 - DNA, 42 bp, synthetic construct
  source 1..42: mol_type = other DNA
cctattattctgaaaatcaagtttgccttttacagagagaat

SEQ ID NO: 50 - DNA, 42 bp, synthetic construct
  source 1..42: mol_type = other DNA
agtgcccgtataaacggcattttcggtcgaaacgattttttg

SEQ ID NO: 51 - DNA, 42 bp, synthetic construct
  source 1..42: mol_type = other DNA
gtactggtaataagtttcataatcaaaattacaaaataaaca

SEQ ID NO: 52 - DNA, 42 bp, synthetic construct
  source 1..42: mol_type = other DNA
gttccagtaagcgtcgcctccctcagagtaccaacgctaacg

SEQ ID NO: 53 - DNA, 42 bp, synthetic construct
  source 1..42: mol_type = other DNA
gcctgtagcattcccaacatataaaagagcagtatgttagca

SEQ ID NO: 54 - DNA, 42 bp, synthetic construct
  source 1..42: mol_type = other DNA
ctcattaaagccagagccaccaccctcatagttgctattttg

SEQ ID NO: 55 - DNA, 42 bp, synthetic construct
  source 1..42: mol_type = other DNA
caggtcagacgattcgccgccagcattgacctcccgacttgc

SEQ ID NO: 56 - DNA, 42 bp, synthetic construct
  source 1..42: mol_type = other DNA
gtcaatagataatacaactcgtattaaaaaggcttatccggt

SEQ ID NO: 57 - DNA, 42 bp, synthetic construct
  source 1..42: mol_type = other DNA
taaaatatctttagaagtttgagtaacaaggaatcattaccg

SEQ ID NO: 58 - DNA, 42 bp, synthetic construct
  source 1..42: mol_type = other DNA
agttggcaaatcaacagaaggagcggaacgcactcatcgaga

SEQ ID NO: 59 - DNA, 42 bp, synthetic construct
  source 1..42: mol_type = other DNA
ttgctgaacctcaaatggcaattcatcagtctttccttatca

SEQ ID NO: 60 - DNA, 42 bp, synthetic construct
  source 1..42: mol_type = other DNA
acgctgagagccagtctgaataatggaaatcctaatttacga

SEQ ID NO: 61 - DNA, 42 bp, synthetic construct
  source 1..42: mol_type = other DNA
agaggtgaggcggtttgcacgtaaaacatatcaacaatagat

SEQ ID NO: 62 - DNA, 42 bp, synthetic construct
  source 1..42: mol_type = other DNA
gccattaaaaatacgtttaacgtcagatgacaataaacaaca

SEQ ID NO: 63 - DNA, 42 bp, synthetic construct
  source 1..42: mol_type = other DNA
gctattagtctttacgggagaaacaataataaagtaccgaca

SEQ ID NO: 64 - DNA, 42 bp, synthetic construct
  source 1..42: mol_type = other DNA
tgtaccgtaacactttttgtcacaatcaggaatacccaaaag

SEQ ID NO: 65 - DNA, 42 bp, synthetic construct
  source 1..42: mol_type = other DNA
tgaaagcgtaagaaaagttacaaaatcggtaatttaggcaga

SEQ ID NO: 66 - DNA, 42 bp, synthetic construct
  source 1..42: mol_type = other DNA
aaaagggacattctcctgagcaaaagaatagggcttaattga

SEQ ID NO: 67 - DNA, 42 bp, synthetic construct
  source 1..42: mol_type = other DNA
ttaccagtataaagcggtaatcgtaaaaatcggtgcgggcct

SEQ ID NO: 68 - DNA, 42 bp, synthetic construct
  source 1..42: mol_type = other DNA
ctagaaaaagcctgtgataatcagaaaagcgccattcgccat

SEQ ID NO: 69 - DNA, 42 bp, synthetic construct
  source 1..42: mol_type = other DNA
aaataaggcgttaaaaatatttaaattgccagctttccggca

SEQ ID NO: 70 - DNA, 42 bp, synthetic construct
  source 1..42: mol_type = other DNA
ttctgacctaaattattaaatttttgttggggacgacgacag

SEQ ID NO: 71 - DNA, 42 bp, synthetic construct
  source 1..42: mol_type = other DNA
acgcgagaaaacttacgccatcaaaaatggtgtagatgggcg

SEQ ID NO: 72 - DNA, 42 bp, synthetic construct
  source 1..42: mol_type = other DNA
aaggaaaccgaggacgtcataaatattcaactaatgcagata

SEQ ID NO: 73 - DNA, 42 bp, synthetic construct
  source 1..42: mol_type = other DNA
ctatatgtaaatgcctttcatcaacattgggaacaaacggcg

SEQ ID NO: 74 - DNA, 42 bp, synthetic construct
  source 1..42: mol_type = other DNA
ctttttaagaaaaggttcagaaaacgagggtagaaagattca

SEQ ID NO: 75 - DNA, 42 bp, synthetic construct
  source 1..42: mol_type = other DNA
agcaagaaacaatgtaccctgactattaatctacgttaataa

SEQ ID NO: 76 - DNA, 42 bp, synthetic construct
  source 1..42: mol_type = other DNA
agagagataacccaaaagattaagaggaaagaactggctcat

SEQ ID NO: 77 - DNA, 42 bp, synthetic construct
  source 1..42: mol_type = other DNA
aacaccctgaacaataattcgagcttcataatttcaacttta

SEQ ID NO: 78 - DNA, 42 bp, synthetic construct
  source 1..42: mol_type = other DNA
aacataaaaacaggacaggtcaggattagagaaacaccagaa

SEQ ID NO: 79 - DNA, 42 bp, synthetic construct
  source 1..42: mol_type = other DNA
tttaacgtcaaaaaaagaggtcatttttcgtaacaaagctgc

SEQ ID NO: 80 - DNA, 42 bp, synthetic construct
  source 1..42: mol_type = other DNA
gccatattatttattataatgctgtagcgagtaatcttgaca

SEQ ID NO: 81 - DNA, 42 bp, synthetic construct
  source 1..42: mol_type = other DNA
agcgtctttccagatacggtgtctggaacggtgtacagacca

SEQ ID NO: 82 - DNA, 42 bp, synthetic construct
  source 1..42: mol_type = other DNA
cacccagctacaataattctgcgaacgataagggaaccgaac

SEQ ID NO: 83 - DNA, 42 bp, synthetic construct
  source 1..42: mol_type = other DNA
gggaggttttgaagttcgcaaatggtcactccatgttactta

SEQ ID NO: 84 - DNA, 42 bp, synthetic construct
  source 1..42: mol_type = other DNA
attctaagaacgcggggcgcgagctgaattgtatcatcgcct

SEQ ID NO: 85 - DNA, 42 bp, synthetic construct
  source 1..42: mol_type = other DNA
cgcccaatagcaagtagcattacatccacggggagaggcggt

SEQ ID NO: 86 - DNA, 42 bp, synthetic construct
  source 1..42: mol_type = other DNA
acaagcaagccgttagcaaaattaagcagaaacctgtcgtgc

SEQ ID NO: 87 - DNA, 42 bp, synthetic construct
  source 1..42: mol_type = other DNA
ttccaagaacgggtggttgtaccaaaaatcacattaattgcg

SEQ ID NO: 88 - DNA, 42 bp, synthetic construct
  source 1..42: mol_type = other DNA
gcatgtagaaaccaagaagcctttatttgcataaagtgtaaa

SEQ ID NO: 89 - DNA, 42 bp, synthetic construct
  source 1..42: mol_type = other DNA
aactggcatgattatagtaaaatgtttaagtaagagcaacac

SEQ ID NO: 90 - DNA, 42 bp, synthetic construct
  source 1..42: mol_type = other DNA
aagtcctgaacaagctcatatattttaaattgttatccgctc

SEQ ID NO: 91 - DNA, 42 bp, synthetic construct
  source 1..42: mol_type = other DNA
tgttcagctaatgcagattcaaaagggtgctcgaattcgtaa

SEQ ID NO: 92 - DNA, 42 bp, synthetic construct
  source 1..42: mol_type = other DNA
aaaggtaaagtaatatcaatatgatatttgcatgcctgcagg

SEQ ID NO: 93 - DNA, 42 bp, synthetic construct
  source 1..42: mol_type = other DNA
ggcattttcgagccggagagggtagctattcccagtcacgac

SEQ ID NO: 94 - DNA, 42 bp, synthetic construct
  source 1..42: mol_type = other DNA
gaatcgccatatttgtcattgcctgagagggatgtgctgcaa

SEQ ID NO: 95 - DNA, 42 bp, synthetic construct
  source 1..42: mol_type = other DNA
ctgaggcttgcaggaggcttgccctgacgagagtacctttaa

SEQ ID NO: 96 - DNA, 42 bp, synthetic construct
  source 1..42: mol_type = other DNA
aaatcggaaccctagtaacgccagggtttttttgagagatct

SEQ ID NO: 97 - DNA, 42 bp, synthetic construct
  source 1..42: mol_type = other DNA
atatgtaccccggttttagtatcatatgaacaatttcatttg

SEQ ID NO: 98 - DNA, 42 bp, synthetic construct
  source 1..42: mol_type = other DNA
aagattgtataagcataagaataaacactaaatcaatatatg

SEQ ID NO: 99 - DNA, 42 bp, synthetic construct
  source 1..42: mol_type = other DNA
ttgttaaaattcgctaatggtttgaaatgtcgctattaatta

SEQ ID NO: 100 - DNA, 42 bp, synthetic construct
  source 1..42: mol_type = other DNA
tttaaccaataggatttcaaatatatttgcgatagcttagat

SEQ ID NO: 101 - DNA, 42 bp, synthetic construct
  source 1..42: mol_type = other DNA
cttcctgtagccagtgatgcaaatccaatttatcaaaatcat

SEQ ID NO: 102 - DNA, 42 bp, synthetic construct
  source 1..42: mol_type = other DNA
aaatgctttaaacataagcagatagccgcgacattcaaccga

SEQ ID NO: 103 - DNA, 42 bp, synthetic construct
  source 1..42: mol_type = other DNA
aaaaatcaggtcttaaatagcaatagctaaattattcattaa

SEQ ID NO: 104 - DNA, 42 bp, synthetic construct
  source 1..42: mol_type = other DNA
gcggattgcatcaacaagaattgagttagccatttgggaatt

SEQ ID NO: 105 - DNA, 42 bp, synthetic construct
  source 1..42: mol_type = other DNA
ggaagcaaactccagaagcgcattagacatagcagcaccgta

SEQ ID NO: 106 - DNA, 42 bp, synthetic construct
  source 1..42: mol_type = other DNA
ttgctccttttgattgaaaatagcagccttagcgtcagactg

SEQ ID NO: 107 - DNA, 42 bp, synthetic construct
  source 1..42: mol_type = other DNA
gcttaattgctgaacccaatccaaataaatagcccccttatt

SEQ ID NO: 108 - DNA, 42 bp, synthetic construct
  source 1..42: mol_type = other DNA
atatgcaactaaaggcctaatttgccagtcaccggaaccaga

SEQ ID NO: 109 - DNA, 42 bp, synthetic construct
  source 1..42: mol_type = other DNA
aacagttgattccctttatcctgaatctccgccaccctcaga

SEQ ID NO: 110 - DNA, 42 bp, synthetic construct
  source 1..42: mol_type = other DNA
gccagagggggtaaagactccttattacaacgcaaagacacc

SEQ ID NO: 111 - DNA, 42 bp, synthetic construct
  source 1..42: mol_type = other DNA
tatattttcatttgaggcgttttagcgaacaggagtagactt

SEQ ID NO: 112 - DNA, 42 bp, synthetic construct
  source 1..42: mol_type = other DNA
tctactaatagtagcaaatcagatatagtcctttgcccgaac

SEQ ID NO: 113 - DNA, 42 bp, synthetic construct
  source 1..42: mol_type = other DNA
caaggcaaagaatttttattttcatcgtttatcattttgcgg

SEQ ID NO: 114 - DNA, 42 bp, synthetic construct
  source 1..42: mol_type = other DNA
cataaagctaaatcattaaaccaagtacttatcatcatattc

SEQ ID NO: 115 - DNA, 42 bp, synthetic construct
  source 1..42: mol_type = other DNA
aatacttttgcgggatcaataatcggctatataatcctgatt

SEQ ID NO: 116 - DNA, 42 bp, synthetic construct
  source 1..42: mol_type = other DNA
taatgtgtaggtaaagaacgcgcctgttgaaataaagaaatt

SEQ ID NO: 117 - DNA, 42 bp, synthetic construct
  source 1..42: mol_type = other DNA
acagtcaaatcacctctgtccagacgacgaatatacagtaac

SEQ ID NO: 118 - DNA, 42 bp, synthetic construct
  source 1..42: mol_type = other DNA
gataaattaatgccagtaataagagaatacggattcgcctga

SEQ ID NO: 119 - DNA, 42 bp, synthetic construct
  source 1..42: mol_type = other DNA
caatactgcggaataacgcaataataacatagaaaattcata

SEQ ID NO: 120 - DNA, 42 bp, synthetic construct
  source 1..42: mol_type = other DNA
acaaaggctatcagaacaacgccaacatcgcagaggcgaatt

SEQ ID NO: 121 - DNA, 42 bp, synthetic construct
  source 1..42: mol_type = other DNA
agagaatcgatgaaccaacgctcaacaggatgatgaaacaaa

SEQ ID NO: 122 - DNA, 42 bp, synthetic construct
  source 1..42: mol_type = other DNA
gaaaggagcgggcgtgttgggaagggcgctagcatgtcaatc

SEQ ID NO: 123 - DNA, 42 bp, synthetic construct
  source 1..42: mol_type = other DNA
acagggcgcgtactagatcgcactccagtaaacgttaatatt

SEQ ID NO: 124 - DNA, 42 bp, synthetic construct
  source 1..42: mol_type = other DNA
cgattaaagggattggataggtcacgttaattcgcgtctggc

SEQ ID NO: 125 - DNA, 42 bp, synthetic construct
  source 1..42: mol_type = other DNA
taattttttcacgtacaacattattacaaatgaccataaatc

SEQ ID NO: 126 - DNA, 42 bp, synthetic construct
  source 1..42: mol_type = other DNA
tgaatttcttaaacccttatgcgattttagcccgaaagactt

SEQ ID NO: 127 - DNA, 42 bp, synthetic construct
  source 1..42: mol_type = other DNA
cggctacagaggctctgaccttcatcaatcaacatgttttaa

SEQ ID NO: 128 - DNA, 42 bp, synthetic construct
  source 1..42: mol_type = other DNA
gcaccaacctaaaacagacggtcaatcagtagatttagtttg

SEQ ID NO: 129 - DNA, 42 bp, synthetic construct
  source 1..42: mol_type = other DNA
agtacaaggtttttccagggtcggagataaggtggcatcaat

SEQ ID NO: 130 - DNA, 42 bp, synthetic construct
  source 1..42: mol_type = other DNA
ggtccacgctggttcgctttccagtcggataaagcctcagag

SEQ ID NO: 131 - DNA, 42 bp, synthetic construct
  source 1..42: mol_type = other DNA
atagcccgagatagcatacgagccggaacaacgcaaggataa

SEQ ID NO: 132 - DNA, 42 bp, synthetic construct
  source 1..42: mol_type = other DNA
ttctgtatgggattgaattacgaggcatgactggatagcgtc

SEQ ID NO: 133 - DNA, 42 bp, synthetic construct
  source 1..42: mol_type = other DNA
aaaaaccgtctatctccccgggtaccgagagaaaggccggag

SEQ ID NO: 134 - DNA, 28 bp, synthetic construct
  source 1..28: mol_type = other DNA
agcgagaggcttttataaaaaccaaaat

SEQ ID NO: 135 - DNA, 28 bp, synthetic construct
  source 1..28: mol_type = other DNA
tagttagcgtaacgacagacagccctca

SEQ ID NO: 136 - DNA, 28 bp, synthetic construct
  source 1..28: mol_type = other DNA
atacataaaggtggaacgtagaaaatac

SEQ ID NO: 137 - DNA, 42 bp, synthetic construct
  source 1..42: mol_type = other DNA
caaatatcgcgtttagtcagagggtaatttaccattagcaag

SEQ ID NO: 138 - DNA, 42 bp, synthetic construct
  source 1..42: mol_type = other DNA
accattagatacatccttaaatcaagatgagccgccaccaga

SEQ ID NO: 139 - DNA, 42 bp, synthetic construct
  source 1..42: mol_type = other DNA
aaatttttagaaccaaaaataatatcccgggttagaacctac

SEQ ID NO: 140 - DNA, 42 bp, synthetic construct
  source 1..42: mol_type = other DNA
gctgcgcgtaaccaggaaaccaggcaaagccccaaaaacagg

SEQ ID NO: 141 - DNA, 42 bp, synthetic construct
  source 1..42: mol_type = other DNA
tgctttcctcgttaatctgccagtttgaaaatcagctcattt

SEQ ID NO: 142 - DNA, 42 bp, synthetic construct
  source 1..42: mol_type = other DNA
ggagtgagaatagaggaataccacattcattgaatccccctc

SEQ ID NO: 143 - DNA, 42 bp, synthetic construct
  source 1..42: mol_type = other DNA
aggagcctttaattcgttgggaagaaaatagtcagaagcaaa

SEQ ID NO: 144 - DNA, 42 bp, synthetic construct
  source 1..42: mol_type = other DNA
tgacaacaaccatcggcttgagatggttaagcgaaccagacc

SEQ ID NO: 145 - DNA, 42 bp, synthetic construct
  source 1..42: mol_type = other DNA
atctaaagttttgttttaccagacgacggcaaaagaagtttt

SEQ ID NO: 146 - DNA, 42 bp, synthetic construct
  source 1..42: mol_type = other DNA
caccctcagcagcgattacccaaatcaagcggatggcttaga

SEQ ID NO: 147 - DNA, 42 bp, synthetic construct
  source 1..42: mol_type = other DNA
agtttccattaaacagaggacagatgaagtttcattccatat

SEQ ID NO: 148 - DNA, 42 bp, synthetic construct
  source 1..42: mol_type = other DNA
actcatctttgaccaaatccgcgacctgataacctgtttagc

SEQ ID NO: 149 - DNA, 42 bp, synthetic construct
  source 1..42: mol_type = other DNA
gattgcccttcaccaatcggccaacgcgataaatcatacagg

SEQ ID NO: 150 - DNA, 42 bp, synthetic construct
  source 1..42: mol_type = other DNA
tggtggttccgaaaatgagtgagctaaccattatgaccctgt

SEQ ID NO: 151 - DNA, 42 bp, synthetic construct
  source 1..42: mol_type = other DNA
gagtccactattaagtttcctgtgtgaaatgcaatgcctgag

SEQ ID NO: 152 - DNA, 42 bp, synthetic construct
  source 1..42: mol_type = other DNA
acccaaatcaagttgccagtgccaagctcaaccgttctagct

SEQ ID NO: 153 - DNA, 42 bp, synthetic construct
  source 1..42: mol_type = other DNA
gggaaagccggcgacagctggcgaaagggtctggagcaaaca

SEQ ID NO: 154 - DNA, 42 bp, synthetic construct
  source 1..42: mol_type = other DNA
acggaataagtttagagtttcgtcaccattagtaaatgaatt

SEQ ID NO: 155 - DNA, 42 bp, synthetic construct
  source 1..42: mol_type = other DNA
tggtttaccagcgccagggatagcaagctcaacagtttcagc

SEQ ID NO: 156 - DNA, 42 bp, synthetic construct
  source 1..42: mol_type = other DNA
ttgagggagggaagcaccctcagaaccgggaattgcgaataa

SEQ ID NO: 157 - DNA, 42 bp, synthetic construct
  source 1..42: mol_type = other DNA
aggtgaattatcaccaccgtactcaggaaaaaaggctccaaa

SEQ ID NO: 158 - DNA, 42 bp, synthetic construct
  source 1..42: mol_type = other DNA
agagccagcaaaatcgtcgagagggttggcttgctttcgagg

SEQ ID NO: 159 - DNA, 42 bp, synthetic construct
  source 1..42: mol_type = other DNA
gccggaaacgtcactaggattagcggggagttgcgccgacaa

SEQ ID NO: 160 - DNA, 42 bp, synthetic construct
  source 1..42: mol_type = other DNA
atcagtagcgacagacatgaaagtattagatatattcggtcg

SEQ ID NO: 161 - DNA, 42 bp, synthetic construct
  source 1..42: mol_type = other DNA
tagcgcgttttcatcagttaatgcccccttttgcgggatcgt

SEQ ID NO: 162 - DNA, 42 bp, synthetic construct
  source 1..42: mol_type = other DNA
agcgtttgccatctttttaacggggtcaaacgagggtagcaa

SEQ ID NO: 163 - DNA, 42 bp, synthetic construct
  source 1..42: mol_type = other DNA
gccaccaccggaaccatacatggcttttctttttcatgagga

SEQ ID NO: 164 - DNA, 42 bp, synthetic construct
  source 1..42: mol_type = other DNA
accgccaccctcagaatggaaagcgcagatgccactacgaag

SEQ ID NO: 165 - DNA, 42 bp, synthetic construct
  source 1..42: mol_type = other DNA
accaccaccagagcggccttgatattcagaatacactaaaac

SEQ ID NO: 166 - DNA, 42 bp, synthetic construct
  source 1..42: mol_type = other DNA
tacaaacaattcgacatttgaggatttacaagcgcgaaacaa

SEQ ID NO: 167 - DNA, 42 bp, synthetic construct
  source 1..42: mol_type = other DNA
gttattaattttaagagcactaacaactgacgggcaacagct

SEQ ID NO: 168 - DNA, 42 bp, synthetic construct
  source 1..42: mol_type = other DNA
aacaaagaaaccaccagttgaaaggaatgagttgcagcaagc

SEQ ID NO: 169 - DNA, 42 bp, synthetic construct
  source 1..42: mol_type = other DNA
ctgattatcagatgatatcaaaccctcaaaaatcctgtttga

SEQ ID NO: 170 - DNA, 42 bp, synthetic construct
  source 1..42: mol_type = other DNA
gtttggattatactcagcaaatgaaaaatataaatcaaaaga

SEQ ID NO: 171 - DNA, 42 bp, synthetic construct
  source 1..42: mol_type = other DNA
catatcaaaattatcagtattaacaccgccagtttggaacaa

SEQ ID NO: 172 - DNA, 42 bp, synthetic construct
  source 1..42: mol_type = other DNA
gcgtagattttcagcgaacgaaccaccaaacgtcaaagggcg

SEQ ID NO: 173 - DNA, 42 bp, synthetic construct
  source 1..42: mol_type = other DNA
agtaccttttacatatgcgcgaactgatctacgtgaaccatc

SEQ ID NO: 174 - DNA, 42 bp, synthetic construct
  source 1..42: mol_type = other DNA
ttgctttgaatacctacgtggcacagactgccgtaaagcact

SEQ ID NO: 175 - DNA, 42 bp, synthetic construct
  source 1..42: mol_type = other DNA
attcatttcaattaggccaacagagatatttagagcttgacg

SEQ ID NO: 176 - DNA, 42 bp, synthetic construct
  source 1..42: mol_type = other DNA
catcaagaaaacaacagattcaccagtcgaagggaagaaagc

SEQ ID NO: 177 - DNA, 42 bp, synthetic construct
  source 1..42: mol_type = other DNA
aattaccttttttacattttgacgctcaagtgtagcggtcac

SEQ ID NO: 178 - DNA, 42 bp, synthetic construct
  source 1..42: mol_type = other DNA
tgagtgaataacctcgccagccattgcacttaatgcgccgct

SEQ ID NO: 179 - DNA, 42 bp, synthetic construct
  source 1..42: mol_type = other DNA
attttcccttagaatcaaactatcggccgagcacgtataacg

SEQ ID NO: 180 - DNA, 42 bp, synthetic construct
  source 1..42: mol_type = other DNA
taagacgctgagaattctttgattagtagctaaacaggaggc

SEQ ID NO: 181 - DNA, 42 bp, synthetic construct
  source 1..42: mol_type = other DNA
aggtctgagagactaaagagtctgtccatacgccagaatcct